(19) United States
(12) Patent Application Publication
(10) Pub. No.: US 2022/0331557 A1
(43) Pub. Date: Oct. 20, 2022

(54) Drive-Enhanceable Handheld Elongate Medical Device Advancer and Related Systems, Devices and Methods

(71) Applicant: New Wave Endo-Surgical Corp., Coconut Creek, FL (US)

(72) Inventors: Ricardo Alexander Gomez, Lighthouse Point, FL (US); Salvatore Castro, Coconut Creek, FL (US); Christian Abreu, Coconut Creek, FL (US); Juan Diaz, Coconut Creek, FL (US)

(73) Assignee: New Wave Endo-Surgical Corp., Coconut Creek, FL (US)

(21) Appl. No.: 17/709,007

(22) Filed: Mar. 30, 2022

Related U.S. Application Data
(60) Provisional application No. 63/167,918, filed on Mar. 30, 2021.

(51) Int. Cl. A61M 25/01; A61B 17/00

(52) U.S. Cl. CPC A61M 25/0113; A61B 17/00234; A61B 2017/00292; A61M 2205/586

(57) ABSTRACT

A drive-enhanceable handheld advancer is provided for advancing an elongate device through a pathway defined in an advancer body that includes a rotatable manual thumbwheel partially embedded in the body for receiving a user-exerted manual control movement from a thumb, and a manual drive having a nip, a transmission, and a reverse clutch. The transmission operatively connects the thumbwheel to the nip for driving advancement, and the reverse clutch maintains a first interference grip connection between the nip and the elongate device. The manual control thumbwheel can move inward responsive to user-applied grip movements to increase grip with the elongate device. The reverse clutch applies augmented force from grip movements to the nip for increasing the grip drive connection, which can be applied at a different angle from the inward movements. The reverse clutch can include slotted pivot supports for the thumbwheel and drive rollers on the advancer body.

### CROSS-REFERENCE TO RELATED APPLICATIONS

This application is related to commonly owned U.S. Pat. No. 9,986,987 to Patel et al. entitled “Apparatus and Method for Fascial Closure Device for Laparoscopic Trocar Port Site and Surgery”), and to U.S. patent application Ser. No. 17/368,825 filed Nov. 5, 2021 entitled “Handheld Elongate Medical Device Advancer and Related System, Devices and Methods.” In addition, this application claims priority to U.S. provisional patent application No. 63/167,918 filed Mar. 30, 2021, entitled “Introducer/Advancer for Elongate Medical Devices and Related Systems, Devices and Methods.” The disclosures of the above applications are incorporated herein by reference in their entirety.

### BACKGROUND

The present application and related subject matter discussed herein relate to medical tools and particularly to surgical instruments, and more particularly relate to an elongate device introducer/advancer surgical support apparatus, such as an elongate device advancer, an introducer for a catheter or a feeding tube, and/or to an introducer/advancer for various elongate medical devices including elongate devices for use with endoscopic tools (generally referred to herein as elongate device advancers). Further, the embodiments described herein relate to an elongate device advancer configured to support laparoscopic surgical instruments with performance of minimally invasive surgery (MIS) functions. The laparoscopic surgical instruments can include a wide variety of MIS surgical devices. However, aspects, features and related concepts are discussed herein along with describing example medical devices and related techniques, such as for instance, procedures and device that can be used with intra-abdominal suturing devices, port closure devices, suture placement devices and the like configured for closing puncture wounds generated by surgical laparoscopic trocar ports and other puncturing devices.

For discussion purposes and example representations to assist with describing aspects, features and inventive concepts herein, reference is made to laparoscopic surgical instruments described in U.S. Pat. No. 9,986,987 to Patel et al. (herein “The Patel Patent”). The Patel Patent describes various configurations of an apparatus and related methods for treating tissue openings, such as an endoscopic trocar port opening created and used for a minimally invasive surgical procedure, including a suture placement device configured for rapidly, safely, efficiently and effectively closing tissue defects created to access the intra-abdominal cavity during laparoscopic surgical procedures. The device as generally described and schematically represented herein can obtain adequate tissue adjacent to the tissue defect for providing a strong closure along with maintaining the pneumoperitoneum during the closure process, which includes techniques involving removal of the trocar from the port opening prior to placement of a fascial suture therein.

Despite the demonstrated effectiveness, reliability and other significant advantages proven by the suture placement device of the Patel Patent, concerns exist regarding potential leakage of gas around the cannula and loss of pneumoperitoneum during suture placement with the trocar removed along with loss of related gas sealing benefits. This concern can create challenges for performing suture placement and closing the wound while exercising extreme caution regarding movements of the suture placement device within the port that could exacerbate gas leakage and potential loss of pneumoperitoneum. Further, there are challenges with the use of suture loading devices described by The Patel Patent for supporting the suture placement device to route a suture through the suture path and close the port. In addition, continual challenges persist with respect to the duration of surgeries and the likelihood of complications increasing with duration, for which effective time-saving improvements and aids for improving efficiencies are consistently desired and pursued.

Known improvements and solutions for supporting suture placement devices like the device of the Patel Patent include guide wire advancement devices that advance a guide wire manually or advance it in a generally slow, stepwise automated manner—neither of which enhance efficiency, reduce suture timing, or improve suture and port closure procedures. Further, many conventional guide wire advancement devices used for orthoscopic and laparoscopic surgical procedures create challenges and introduce concerns that weigh against their usage with these types of surgical devices versus simple manual advancement devices. Due to their small size and precise maneuverability requirements, such devices have complex and sometimes tortious pathways for a guide wire to traverse quickly and easily, much less can easily be driven by an elongate device advancer, which can be especially true for port closure and fascial suture devices and procedures.

Many conventional elongate device advancers are commonly used with complex devices, surgical procedures and maneuvers, such as cardiac procedures like angioplasty or implant deployment, spinal surgery manipulations and procedures, complex routing for imaging functions, and even procedures for guiding catheters into blood vessels. These advancer devices are large, complex devices as necessitated by the complex functionality they are primarily configured to perform. Such conventional elongate device advancers are ineffective at supporting relatively small surgical devices and use with less invasive procedures including laparoscopic port closure procedures. The use of such suture placement devices can greatly enhance the effectiveness of the sutures and timing for implementing the sutures. However, guide wire routing and advancement struggles continue to impede the usage and full realization of their benefits. Similarly, the use of smaller, maneuverable elongate device advancers for introducing and/or advancing other elongate medical devices including tubes and catheters can enhance likewise enhance the effectiveness and timing for implementing related procedures. However, ineffective conventional elongate device advancers often increase risks and raise additional challenges when used for supporting other surgical devices, such as gas leakage or loss of the pneumoperitoneum associated with performing port closure functions.

Small, simple conventional elongate medical devices are known that are configured for manual operation including being operable with relative case by a surgeon, such as by a single hand, which can be configured for use with port placement devices. However, there are drawbacks associated with using conventional elongate medical devices to support a suture placement device like the apparatus for port closure described in the Patel Patent. For example, these conventional devices require the surgeon to perform repeated, time-consuming, manual drive actions or require the surgeon to make movements or perform actions that increase risks.

For instance, FIG. 1A shows a simple, conventional elongate device advancer 10 formed as a guide wire combined with a sheath coil 10. The sheath coil 10 includes an integrated manual advancement notch 12 formed through the sheath, which is described further in U.S. Patent No. 5,810,12 to Lynch et al. entitled, “Guidewire Advancement System.” As shown in FIG. 1B, advancement notch 12 requires the surgeon to manually push the guide wire using a thumb or finger through the sheath notch 12, which is difficult and cumbersome to use with a laparoscopic surgical device when installed in a patient, such as a suture placement device. Further, such conventional advancer devices necessitate numerous repetitive advancement movements that slows port closure procedures and thus provides little assistance, if any, compared with manually pushing bare guide wire through the suture placement device.

As further example, FIG. 2 shows another conventional manual advancement device that is representative of similar manual devices. Manual advancement device 110 is coupled with a guide wire within a sheath at a proximal end like advancement device 10, and includes a stylet guide at its distal end for improving alignment and coupling with a surgical device, such as a suture placement device. However, like advancement device 10, manual advancement device 110 requires the surgeon to perform numerous, repetitive advancement actions by repeatedly advancing and retracting shuttle 116. While advancement device 110 improves coupling of the advancement device with the suture placement device, it nonetheless is slow and cumbersome for efficient usage with such a surgical device—particularly for placing multiple sutures and closing multiple ports in a patient within a short period of time.

Referring now to FIGS. 3A-3E, a diagrammatic plan view of a minimally invasive surgical environment is shown that illustrates significant benefits and advantages that can be provided via the use of an effective suture placement device 50 for port closure procedures, as well as showing drawbacks associated with usage of conventional elongate device advancers in combination with the same. As shown in FIG. 3A, several small diameter ports are typically created through patient tissue for laparoscopic surgical procedures, such as intra-abdominal surgeries. These ports are often formed through the patient's skin 20 (i.e., abdominal skin), fat layer 22, through a fascial layer 24, and sometimes muscles, which are each kept open as a laparoscopic port via use of a trocar port device 85. The port depth A is determined and a trocar port device 85 is selected to maintain the port during surgery along with a corresponding suture placement device 50 for closing the same.

The trocar port device 85 permits access for surgical instruments, orthoscopic cameras, and the like during surgery while simultaneously sealing the port to prevent the loss of inert gas. Inert gas, such as carbon dioxide, is typically pumped in through one or more ports to create a pneumoperitoneum balloon or space below the skin and above the surgical area to provide vital viewability for surgical procedures and maneuver space for surgical instruments and performing procedures. At the conclusion of the surgical procedures, these ports require effective suturing to close the corresponding wound and prevent herniation, which is best performed while maintaining the pneumoperitoneum and starting with effective placement of sutures at the fascial layer to close the port from the inside out.

Suture placement device 50 greatly enhances the ability of a surgeon to effectively place sutures starting at the fascial layer 24. The approach of effectively placing fascial sutures first and moving outward has been shown to enhance healing, reduce pain, and greatly reduce the possibility that the port will reopen. An effective method for placing sutures using a suture placement device 50 is illustrated in FIGS. 3A-3E, which includes inserting 62 the distal end of an elongated cannula of the suture placement device 50 through the corresponding port such that its distal end extends beyond the distal end of the trocar port device 85, followed by withdrawing 64 the trocar port device 85 over the suture placement device 50 as illustrated in FIG. 3A. The surgeon will select a suture placement device 50 having a diameter slightly less than the inner diameter of the trocar port device 85, such that suture placement device 50 is able to maintain the port opening and prevent significant gas leakage until sutures are placed and the port is closed. The suture placement device 50 is selected for the port such that its elongate cannula has a longitudinal length B sufficient for extending internally beyond the distal end of the trocar port device 85, spanning the length of the port, and extending proximally an appropriate length for the surgeon to maintain control of the suture placement device and effectively use it for suture procedures.

The suture placement device 50 is used to position a suture for intra-abdominal suturing and suture puncture wounds generated by surgical laparoscopic trocar ports and other puncturing devices, and to do so without any exposed sharps, which is enabled due to the suture placement device creating the suture path within the device and the suture being loaded therethrough. This is accomplished, in part, via rotation 66 of a pivot bar or ‘T-bar’ disposed at the distal end of the suture placement device 50 about ninety degrees from its longitudinal orientation during insertion, such that the pivot bar is substantially parallel with the fascia layer 24 and skin 20, and extends across and beyond the width of the port as shown in FIG. 3B. Thereafter, the suture placement device 50 is withdrawn 68 externally until the top portions of the pivot bar are in contact with the fascia layer 24.

Referring to FIGS. 3C and 3D, stylet guides disposed on opposite lateral sides of the cannula of the suture placement device 50 are pushed downward 70 or extended distally 70 through the fascia (and muscle as appropriate) until each stylet guide connects with and extends into corresponding openings formed in the rotated pivot bar. The elongate cannula of the suture placement device, the pair of stylet guides, and the pivot bar each define channel segments therein. Upon connection of each stylet guide with and extending into the corresponding openings formed in the rotated pivot bar, the internal channel segments connect to form an uninterrupted internal channel pathway 80 within the suture placement device that extends through the fascia layer along a desired suture path.

As can be seen in FIG. 3D, the internal channel pathway defined through the suture placement device 50 extends from an entry port 81 formed at a proximal portion of the device longitudinally downward or distally within a first channel formed in the elongate cannula of the suture placement device to and through a first one of the stylet guides. The channel pathway 80 continues uninterrupted around and through the rotated pivot bar at the distal end of suture placement device turning into and upwardly or proximally through the second one of the stylet guides. The channel pathway continues proximally through a second channel formed in the elongate cannula to an exit port 83 formed at the proximal portion of the device. Thus, once suture placement device 50 has been placed or installed within a port to be closed, and has been prepared for placement of a fascial suture or other suture, the suture placement device 50 defines therethrough an uninterrupted channel pathway 80 along a desired suture path.

The use of a guide wire to traverse the channel pathway 80 can greatly enhance the placement and completion of sutures using the suture placement device 50. An appropriate guide wire efficiently and effectively advanced through the channel pathway can significantly shorten the time spent placing a suture using the device. In particular, this can be the case when a corresponding or matching guide wire is selected for the length LCP (see FIG. 3E) of the channel pathway 80, its internal diameter, and corresponding parameters of the guide wire, which can more effectively be advanced through the channel pathway according to the matching parameter. Such a guide wire can be advanced or threaded through the channel pathway from the entry port 81 to the exit port 83 and, thus be used to pull a suture thread attached to its proximal end through the channel pathway 80 for quick and efficient suturing. However, drawbacks continue to persist for effectively and efficiently advancing the guide wire through the suture placement device.

After the suture thread has been directed through the channel pathway and extends along the pathway, the suture thread is in place to form a highly effective suture through the fascia layer 24 for closing the port. Additional sutures can further be placed through the fascia layer as needed, such as for irregularly shaped or large ports by rotating suture placement device 50 along its longitudinal axis within the port and rotate the pivot bar a desired amount, such as ninety degrees, to place an additional suture in a similar manner. Once a suture thread is placed along the channel pathway, thin lateral slots along the stylet guides and pivot bar allow the suture thread to slide out of the channel pathway 80 and the suture placement device 50 while maintaining the desired placement through the fascia layer to establish the suture. The stylet guides can be withdrawn upward or proximally, and the pivot bar can be rotated back to its initial elongate position to facilitate the suture thread withdrawing from the device while maintaining its suture position, as well as partial or complete proximal withdrawal of the suture placement device 50 out of the port as appropriate for releasing and completing the suture. Thereafter, a suture can be tightened and tied off to close the port at the fascia layer.

It is understood that the same, related, or similar surgical devices including configurations of other suture placement devices could also be used for discussion and description purposes with respect to inventive features discussed herein, as well as for identifying disadvantages of conventional elongate device advancers discussed in the context of assisting the suture placement device 50. For example, various suture placement devices exist that include different structural elements, operate in differ manners, and employ different methods for creating suture path segments and/or even complete channel pathways. Nonetheless, disadvantages and/or shortcomings of conventional advancers likewise exist for assisting similar and different types of surgical devices, such as time-consuming, repetitive motion advancers and/or complex, cumbersome advancers configured for use with complex surgical devices and procedures, which are ineffective for use with relatively small laparoscopic surgical devices and the like.

In addition, aspects and features of example device arrangements described herein nonetheless apply to a wide variety of surgical devices including various suture placement devices, introducers and/or advancer devices for use with guide wires as well as with other elongate medical devices, such as percutaneous tubes, catheters including dual lumen balloon catheters and single lumen catheters, guide wires and other elongate tubes and devices, and are not limited to use with the example suture placement device or guide wires. For example, other suture placement devices may begin or end their channel pathways at different locations, such as at a middle portion of a cannula, or may require the use of two guide wires to be thread through a pair of partial channel pathways, as well as more significant variations. Many conventional elongate device advancers are designed for use with complex surgical devices and are some are even integrated therewith. Such devices are thereby overly cumbersome or difficult for use as an assist device for small, relatively simple surgical devices like with suture placement devices. Other conventional elongate device advancers that are designed for use with small and/or relatively simple surgical devices are slow, require repetitive manual controls, and/or tend to enhance risk or unduly complicate procedures such that benefits provided rarely outweigh the risks, ineffective operations, and/or other disadvantages—particularly for deployment of a guide wire along a non-interrupted pathway and/or having a determined or desired length for deployment of the guide wire.

Thus, although aspects and features described herein provide significant benefits and advantages for usage with a suture placement device and/or even the configuration of a suture placement device described as an example for discussion purposes including configurations of the Patel device, it is understood that the elongate device advancer device described herein is not so limited. FIGS. 4A, 4B and 5 further illustrate disadvantages of conventional elongate medical device advancers coupled with the example suture placement device and the like including the Patel device. FIG. 4A illustrates the use of conventional advancer 110 discussed above along with FIG. 2 coupled with the suture placement device 50 as described along with FIGS. 3A-3E. Although conventional elongate device advancer 110 may be able to advance a guide wire through the channel pathway 80 of suture placement device 50, advancer 110 requires many repetitive manual advancement actions by the surgeon to advance the guide wire through the length of the channel pathway 80.

Similarly, FIG. 5 shows usage of yet another conventional elongate device advancer 210 depicted in a usage arrangement with suture placement device 50 to advance an elongate device in the form of a guide wire along the channel pathway 80. Conventional advancer 210 not only requires multiple repetitive actions to be performed by the user, but advancer 210 further requires two hands to operate (or two users—one to maintain the suture placement device and another to advance the guide wire), such that its usage can induce significant movements capable of creating other challenges or enhancing risks to the patient or surgical environment.

Improvements have been proposed for more complex conventional elongate device advancers that attempt to address shortcomings of simple advancement devices, which primarily include complex, electrically powered advancement mechanisms specially designed for particular surgical procedures. While these devices can provide benefits associated with powered drives and customized functions, such devices can significantly increase surgical costs while having their applicability limited according to the customized designs. Proposed improvements also include more complex devices having combined functionality, which can reduce the need for swapping devices and/or introducing additional devices during surgical procedures. However, these devices suffer from design tradeoffs that limit potential features that can help overcome deficiencies and meet needs for versatile, highly effective and efficient introducers/advancers in favor of providing the combination device.

For instance, referring now to FIGS. 6A & 6B, a combination tool 21 is shown in FIG. 6B for performing sinuplasty related functions, which as shown in the cross-sectional side view of FIG. 6B, includes a guidewire and balloon insertion mechanism 150, which are shown and described in further detail in U.S. Patent Publication No. 2019/0038301 to Algawi et al. published Feb. 7, 2019 (“Algawi device”). The Algawi device includes a thumb-driven actuating gearwheel 164 and an intermediate gearwheel 166, which contacts a guidewire when downward pressure is applied on gearwheel 164 toward the guidewire and the thumbwheel is rotated by the user's thumb. Upon release of pressure on the gearwheel 164, the guidewire is freed from contact with intermediate gearwheel 166, such that the guidewire can freely translate with respect to mechanism 150 for enabling angioplasty-related functions of the tool. Thus, potential benefits for the conventional device when freed from integration with the overall tool, such as maintaining continued contact with the advancer for precise control of guidewire position and translation, can be lost between intermittent engagement of the guidewire as needed for combined tool functionality. Further, as can be seen in FIG. 6A, the guidewire and balloon insertion mechanism portion of tool 21 is fixedly retained as part of and in alignment with the overall sinuplasty tool, which further restricts maneuverability and related freedoms for use of the guidewire mechanism as integrated in the overall tool.

As another example, FIG. 7 shows an introducer apparatus 410 of a combination assembly for an Ultrasound-Guided IV Catheter, which is further shown and described in U.S. Pat. No. 10,143,826 to SanoStik, LLC that issued on Dec. 4, 2018 (“SanoStik device”). The SanoStik device includes a guide wheel assembly 610 having a pair of parallel guide wheels 611a and 611b rotatably mounted on a shared axis and spaced apart from each other by a spacer plate that defines a guidewire gap therebetween. A first one of the guide wheels 611a is arranged to rotatably engage a drive wheel attached to a rubber gasket 619, such that rotation of the pair of drive wheels 611a and 611b via a user's thumb in a forward or advancement direction rotatably drives the rubber gasket 619 in an opposite direction. An outer surface of the rubber gasket 619 contacts a side portion of the guidewire 412 and pushes the guide wire in the forward or advancement direction through the guidewire gap formed between the pair of guide wheels 611a and 611b.

Operations of introducer apparatus 410 for precisely controlling translation of the guidewire are limited to forward movements while under uninterrupted control of the user, so as to enable the IV catheter placement functionality and avoid related potential complications during such procedures that can occur with exposure to blood and fluid during reverse translation of the guide wire. As such, the introducer apparatus 410 includes a leaf spring 628 for providing tactile notification of forward movement to the user along with locking translation of the guidewire for only permits unidirectional translation in the forward/advancement direction. Further, similar to apparatus 310 described above, introducer apparatus 410 is constrained to an aligned orientation and position as integrated with the combined IV catheter placement apparatus, which limits potential operations and controls of the device.

Conventional elongate device encounter further drawbacks for their lack of grip flexibility or adjustability during use, such as in the event of challenging introducer pathways and obstacles. Bends and other difficult regions of pathway for advancing a guidewire, catheter or other elongate medical device often cause advancers to slip and delay procedures. Conventional advancers have preset grip connections for the particular elongate device and/or maintain an initial grip connection throughout usage without options for modifying without restarting advancement.

Accordingly, there is a need for a simple elongate device advancer configured to be held in and easily operated by a single hand of a user as an assist device for usage with a small, relatively simple surgical device, such as a suture placement device including the Patel device, as an infusion catheter introducer/advancer, and/or an introducer/advancer usable for different types of elongate medical devices and for a wide variety of procedures. Further, there is a need for such a device that is easily operable by the user for imparting introducer/advancer operations without requiring significant control movements by the user or multiple manual advancement actions, and for providing precise continuous control over the position and translation of an elongate device being introduced into the body or advanced along a surgical path, as well as for an easy-to-manipulate & control, manually driven advancer capable of providing continuous direct control and translation of an elongate device, and/or amplified translation of an elongate device.

Further, a need exists for an elongate device advancer that can readily deploy a guide wire through a channel pathway having a determined length including deploying the guide wire through a substantial portion of the determined length. In addition, a need exists for an elongate device advancer configured for use with suture placement device 50 and other similar devices having known parameters, such as a known channel pathway length, diameter, shape, resistance or other matchable parameters, that can provide enhanced customized advancement or deployment along the pathway. In addition, needs exist for an elongate medical device advancer having options or flexibility for modifying a grip connection or augmenting the grip connection for portions of advancement.

### SUMMARY

This summary introduces certain aspects of the embodiments described herein to provide a basic understanding. This summary is not an extensive overview of the inventive subject matter, and it is not intended to identify key or critical elements or to delineate the scope of the inventive subject matter.

One general aspect includes an augmented, anti-slip handheld advancer body defining a pathway for the elongate medical device extending between an inlet at a proximal end portion and an exit at an opposite distal end portion for advancement into an introducer pathway. The augmented advancer also includes a manual control thumbwheel partially embedded in the advancer body, in which the thumbwheel is arranged to receive a user-exerted drive movement from a hand for rotating the thumbwheel about a thumbwheel axis. The augmented advancer also includes a manual drive attached to the advancer body and operatively coupled with the thumbwheel, in which the manual drive includes a nip, a transmission, and a reverse clutch. The manual drive has a first roller and an opposing second roller configured for jointly engaging opposite outer surface regions of the elongate medical device therebetween in an interference fit at a drive location along the pathway. The nip is configured to maintain a constant drive connection with the elongate medical device at the drive location when extending through the pathway and the drive location. The transmission connects the nip with the thumbwheel for transmitting a drive force to the elongate medical device at the drive location responsive to receiving the user-exerted rotational drive movement; and a reverse clutch for maintaining and selectively augmenting a first interference grip connection between the nip and the elongate medical device when the elongate medical device extends through the drive location. The first interference grip connection transmits the drive force to the elongate medical device to advance the elongate device through the advancer and into the introducer pathway without slipping. The reverse clutch selectively increases the first interference grip connection to a greater second interference grip connection between the nip and the elongate medical device responsive to the thumbwheel receiving an inward user-exerted grip increase movement.

Implementations may include one or more of the following features. The augmented, anti-slip handheld advancer the reverse clutch may include: a pair of adjustable thumbwheel rotation supports rotatably connecting the thumbwheel axis with the advancer body, the pair of adjustable thumbwheel supports configured for rotatably supporting the thumbwheel axis at an initial position on the advancer body corresponding with the first interference grip connection, enabling inward movement of the thumbwheel to an augmented grip position responsive to a user-exerted grip increase movement, and for rotatably supporting the thumbwheel axis at the augmented grip position on the advancer body different from the initial position corresponding with the greater second interference grip connection. The thumbwheel is arranged to receive a user-exerted grip increase movement from the thumb including a translation movement for moving the thumbwheel axis from the initial position to the augmented grip position, in which the translation movement is configured to increase compression of the compressible interface and thereby increase the interference grip connection of the nip from the first interference grip connection to the greater second interference grip connection for augmenting the drive force applied to advance the elongate medical device. The reverse clutch may include a pair of adjustable driven rotation supports rotatably connecting an axis of the second roller with the advancer body, and the pair of adjustable driven rotation supports are configured for rotatably supporting the second roller axis at an initial position on the advancer body corresponding with the first interference grip, enabling outward movement of the second roller away from the nip to an augmented grip position for bilaterally augmenting the interference grip, and for rotatably supporting the second roller axis at the augmented grip position on the advancer body different from the initial position corresponding with the greater second interference grip connection.

A compressible interface radially extends about the second roller, and the compressible interface can bias the thumbwheel axis to the initial position for providing increased and bilateral compressive force for augmenting grip with the elongate medical device. The pair of adjustable driver rotation supports may include a first pair of opposing, parallel slotted driver rotation supports oriented inward along the advancer body in a nip tighten direction, the driver slotted rotation supports rotatably supporting the drive roller at the initial position at a first end of the driver slotted rotation supports and at the augmented grip position disposed inward in the nip tighten direction along the driver slotted rotation supports. The pair of adjustable driven rotation supports may include a pair of opposing, parallel slotted driven rotation supports oriented in a nip tighten direction, for which the driven slotted rotation supports rotatably supports the second roller axis at the initial position at a first end of the driven slotted rotation supports and at the augmented grip position is disposed outward away from the nip along the nip tighten direction along the driven slotted pivot supports. The augmented, anti-slip handheld advancer may include: a slot limiter attached to the advancer body for at least one of the driver slotted rotation supports and the driven slotted rotation supports, and the slot limiter can permit the user to at least one of limit a length of the corresponding driver slotted rotation supports or driven slotted rotation supports, or to set a position of the corresponding driver slotted rotation supports or the driven slotted rotation supports. The pair of adjustable thumbwheel rotation supports may include a pair of opposing, parallel slotted thumbwheel rotation supports oriented inward along the advancer body in an increase grip direction, the thumbwheel slotted rotation supports rotatably supporting the thumbwheel axis at the initial position at a first end of the pair of slotted thumbwheel rotation supports and at the augmented grip position disposed inward in the increase grip direction along the thumbwheel slotted rotation supports. The pair of adjustable driver rotation supports may include a pair of opposing, parallel slotted driver rotation supports oriented inward along the advancer body in a nip tighten direction, the driver slotted rotation supports rotatably supporting the first roller axis at the initial position at a first end of the driver slotted rotation supports and at the augmented grip position disposed inward in the nip tighten direction along the driver slotted rotation supports.

The advancer body can define an upper lateral thumb engagement surface at the distal end portion for receiving the user's thumb and for user engagement with an exposed portion of the manual control during use of the advancer. The slotted thumbwheel rotation supports are disposed proximate the lateral thumb engagement surface oriented inward and generally proximally along the advancer body away from the external thumb engagement surface. The increase-grip direction defines an acute angle with a proximal side of the external thumb engagement surface corresponding with flex movement of the user's thumb. The advancer body further can define a lower lateral grip surface at the distal end portion opposite from the upper lateral thumb engagement surface for receiving the user's fingers and gripping the advancer during use; and the nip-tighten direction can define an acute angle with the lower lateral grip surface corresponding with the user's grip. The increase-grip direction can define an acute angle with the nip-tighten direction, and the first roller can translate from the first position to the second position in the nip-tighten direction internally and distally along the advancer body from the first clutch direction. The augmented, anti-slip handheld advancer can include a slot limiter attached to the advancer body for at least one of the thumbwheel and driver slotted rotation supports, and the slot limiter can permit the user to at least one of limit a length of the corresponding thumbwheel or driver slotted rotation supports or to set a position of the corresponding thumbwheel or driver slotted rotation support.

The drive roller further may include a first outer region rotatable with the drive roller. A first outer engagement surface of the first outer region is configured to engage a first external radial portion of the elongate medical device, and the first outer region can include a first compressible material configured to engage the first external radial portion in an interference relationship for the first interference grip, where the first compressible material is compressed during engagement of the first drive roller with the elongate device. The reverse clutch can include a first compressed portion of the first compressible material disposed in the interference relationship for the first interference grip, and the first compressed portion can have a first compressed state for the first interference grip connection and a second compressed state greater than the first compressed state for the second interference grip connection.

The second roller further can include a second outer region rotatable with the second drive roller, and a second outer engagement surface of the second outer region can be configured to engage a second external radial portion of the elongate medical device. The second outer region can include a second compressible material configured to engage the second external radial portion in an interference relationship, where the second compressible material is compressed during engagement of the second drive roller with the elongate device. The reverse clutch can include a third compressed portion of the second compressible material disposed in the interference relationship for the first interference grip connection and a fourth compressed state greater than the third compressed state for the second interference grip connection. The first compressible material can be compressed a greater amount than the second compressible material for engagement of the reverse clutch for the increased second interference grip connection versus the first interference grip connection. The first and the second compressible materials can have substantially the same compressibility, and the second compressible materials have substantially different compressibility properties.

One general aspect can include a handheld advancer for advancing an elongate medical device using a single hand in which the advancer body defines a pathway for the elongate medical device extending between an inlet at a proximal end portion and an exit at an opposite distal end portion. An upper palm engagement surface can have a proximal palm rest and a distal thumb engagement region at an obtuse angle from the palm rest, and a lower finger grip region opposite the upper palm engagement surface and distal thumb engagement region. The upper palm engagement surface and the lower finger grip region can be configured for ergonomic single hand grip of the advancer and user control of the advancer through intuitive distal thumb roll movements for distal advancement of the elongate medical device and through inward application of augmented grip forces for increasing advancer grip with the elongate medical device. The advancer can also include a manual control thumbwheel partially embedded in the advancer body distal end within the thumb engagement region, in which the thumbwheel can be arranged to receive the distal thumb roll movements to advance the elongate medical device distally and receive the inward application of thumb grip forces for enhancing advancer grip with the elongate medical device.

The advancer can also include a manual drive attached to the advancer body and operatively coupled with the thumbwheel, and the manual drive can include: a nip having a first roller and an opposing second roller configured for jointly engaging opposite outer surface regions of the elongate medical device therebetween at a drive location along the pathway. The nip can be configured to maintain a constant translation drive connection with the elongate medical device at the drive location when the elongate medical device extends through the drive location. A transmission can connect the nip with the manual control for transmitting a distal advancement translation force to the elongate medical device at the drive location responsive to user-exerted distal thumb roll movement of the thumbwheel. The advancer can include a reverse clutch for maintaining establishing and selectively augmenting an interference grip connection with the elongate medical device when extending through the nip at the drive location. The first interference grip connection can transmit the drive force to the elongate medical device to advance the elongate device through the advancer without slipping and into the introducer. The reverse clutch can selectively increase the first interference grip connection to a greater second interference grip connection between the nip and the elongate medical device responsive to the thumbwheel receiving an inward user-exerted grip increase movement for avoiding slip conditions, and can be configured to apply the application of grip forces to the nip for increasing the interference grip connection responsive to the user-exerted inward application of grip forces to the thumbwheel. The reverse clutch can also be configured to apply the inward application of grip forces received in a grip direction from the thumbwheel to the nip at a nip-tighten direction angled away from the grip direction.

Implementations may include one or more of the following features. The advancer can include: a pair of thumbwheel rotation slots rotatably supporting the thumbwheel on the advancer body, in which the first pair of pivot slots is oriented in the grip direction substantially perpendicular with the thumb. A pair of driver rotation slots can rotatably support a first roller of the nip on the advancer body, and the pair of driver rotation slots can be oriented in the nip-tighten direction substantially perpendicular to the nip. A movement interface can extend between the thumbwheel and the first roller, which can be configured to move an axis of the first roller in the nip-tighten direction toward the nip when the thumbwheel moves in the grip direction inward away from the thumb, in which the grip direction and the nip-tighten direction form an acute angle therebetween.

One general aspect can include a method for selectively increasing grip between an elongate medical device advancer and an elongate medical device extending through the advancer. The method can also include defining an elongate device guided pathway through an advancer enclosure having a nip for advancing the elongate device operatively coupled with a manually rotatable thumbwheel for driving nip rotations, and the thumbwheel can be arranged to apply inward grip movements to the nip for enhancing interference grip with the elongate device through the nip. The method also includes guiding the elongate device through the pathway including establishing an interference fit between the elongate device and the nip for providing an advancement grip connection with the elongate device. The method further includes rotating the nip for advancing the elongate device responsive to user-exerted thumbwheel rotations for advancing the elongate device. The method also includes concurrent with rotating the nip, selectively tightening the nip interference advancement connection with the elongate device responsive to receiving user-exerted grip movement and forces applied to the thumbwheel along with thumbwheel rotations.

Implementations may include one or more of the following features. The method where: defining the elongate device guided pathway includes arranging the nip and a drive portion of the pathway at a nip-tighten angle substantially perpendicular to the nip at an acute angle from a direction of inward grip movements for the thumbwheel for enabling effective advancement of the elongate device along the pathway along with providing an ergonomic advancer arrangement that can be gripped and controlled by a single hand of the user; establishing the interference fit includes establishing an interference fit that can advance the elongate device through the pathway and introduce the elongate device into a target pathway. Selectively tightening the nip interference advancement connection can include increasing the nip interference connection as needed for traversing the target pathway.

Other medical devices, support devices for medical devices, related components, medical device systems, and/or methods according to embodiments will be or become apparent to one with skill in the art upon review of the following drawings and detailed description. It is intended that all such additional medical devices, related components, medical device systems, and/or methods included within this description be within the scope of this disclosure.

### BRIEF DESCRIPTION OF THE DRAWINGS

FIG. 1A is a plan view of a prior art elongate device advancer device.

FIG. 1B is a close view of the manual advancement portion of the prior art elongate device advancer of FIG. 1A.

FIG. 2 is a plan view of another prior art elongate device advancer device.

FIG. 3A is a diagrammatic plan view of a minimally invasive surgical environment configured to use an example prior art surgical device in the form of a suture placement device that can be supported by aspects and features of elongate device advancers discussed herein, for which the suture placement device and corresponding diagrammatic surgical environment are described and shown in FIGS. 3A-3D for discussion purposes to assist with describing aspects and features described herein; To Wit, the diagrammatic surgical environment shown includes a prior art trocar device installed through a laparoscopic surgery port formed through patient tissue along with the example prior art suture placement device inserted therethrough in preparation for port closure procedures.

FIGS. 3B and 3C are plan views showing example actions pertaining to arranging the suture placement device of FIG. 3A for placement of a suture, for which aspects and features of the elongate device advancer and methods described hereafter can assist and provide benefits and advantages in comparison with conventional elongate device advancers.

FIG. 3D is a plan view showing an arrangement of the suture placement device of FIG. 3A prepared for placement of a suture prior to introduction of an elongate device including having a channel pathway established through the device corresponding with a desired return loop pathway for the suture along with the elongate device in advance of suture material.

FIG. 3E shows an outline of the channel pathway for the arrangement of the example suture placement device illustrated in FIG. 3D along with identifying the length of the channel pathway through which an elongate device can be routed to assist with suture placement.

FIGS. 4A and 4B are plan views showing a conventional device and method for advancing an elongate device through the example suture placement device of FIG. 3D using the prior art advancer of FIG. 2.

FIG. 5 is a plan view showing of another conventional device and method for advancing an elongate device in the form of a guide wire through the suture placement device of FIG. 3D.

FIG. 6A is a perspective view of a combined functionality conventional sinuplasty apparatus including a guide wire introducer for use with sinuplasty procedures, and FIG. 6B is a cross-sectional view of a portion of the conventional guide wire introducer of the sinuplasty apparatus of FIG. 6A.

FIG. 7 is a partially exploded perspective view of another conventional elongate device advancer integrated as part of a combined function IV placement apparatus.

FIG. 8A is a plan view of a diagrammatic representation of a guide wire advancer according to inventive aspects and features described herein, which is shown coupled with a guide wire coil in preparation for use with a medical device, such as the suture placement device of FIGS. 3A-3D.

FIGS. 8B and 8D are diagrammatic plan views of the guide wire advancer representation of FIG. 6 shown prior to and near the end of actuation of the actuator.

FIG. 8C is a close view of diagrammatic portions of the manual drive and the automated drive of the guide wire advancer representation of FIG. 6, which is denoted as View A in FIG. 7A.

FIG. 9 is a diagrammatic plan view of another guide wire advancer representation according to aspects and features described herein illustrating advancement of a guide wire through the suture placement device of FIG. 3D.

FIG. 10 is a schematic representation of a method for advancing a guide wire through the channel pathway of a medical device using the suture placement device of FIG. 3D as an illustrative example along with a diagrammatic representation or example configurations of a guide wire advancer discussed herein, such as the diagrammatic guide wire advancers of FIGS. 6-8 and FIG. 9.

FIG. 11 is a plan view of an example embodiment of a guide wire advancer illustrating advancement of a guide wire through the example suture placement device of FIG. 3D.

FIG. 12 is a left front perspective view of the guide wire advancer of FIG. 11.

FIG. 13 is a right rear perspective view of the guide wire advancer of FIG. 11.

FIG. 14A is a right plan view of the guide wire advancer of FIG. 11 shown with the stylet body transparent to expose the guide wire pathway and portions of the drive system along with drive-related components viewable in the area identified as View B, which is shown in greater detail in FIG. 14C.

FIG. 14B is a rear plan view of the guide wire advancer of FIG. 11.

FIG. 14C is a close view of the portion labelled “View B” in FIG. 14A, which shows a plan view of the nip and a portion of the guide wire pathway for the guide wire advancer of FIG. 11 along with a portion of an example guide wire disposed in the pathway.

FIG. 15A is a left plan view of the guide wire advancer of FIG. 11 shown with the left side cover removed and the left side gearbox support plate transparent exposing some of the drive components of the guide wire advancer of FIG. 11.

FIG. 15B is another left plan view of the guide wire advancer of FIG. 11, but shown with the spring and the left side gearbox support plate removed, as well as the pulley gear transparent to expose components pertaining to the example arrangement of the transmission switch including a movable float gear.

FIGS. 15C-E are close views of the portions labelled as “View 15C/D/E” in FIGS. 15A and 15B, which illustrate example operations of the transmission switch including movements of the float gear.

FIG. 16 is a left plan view similar to FIGS. 15A and B, but without showing housing components including gearbox plates, covers or the stylet body to expose more clearly the example arrangement and interactions of drive components of the guide wire advancer of FIG. 11.

FIGS. 17 and 18 show assembled drive components with and without the gearbox housing components of the guide wire advancer of FIG. 11.

FIG. 19 is an exploded view of drive components of the guide wire advancer of FIG. 11.

FIG. 20 is a schematic representation of a method for advancing a guide wire through the channel pathway of a medical device using the suture placement device of FIG. 3D as an illustrative example along with aspects and features of guide wire advancers discussed herein, such as the guide wire advancer of FIG. 11.

FIG. 21 is a plan view of another example embodiment of a guide wire advancer illustrating advancement of a guide wire through the example suture placement device of FIG. 3D.

FIG. 22 is a left perspective view of the guide wire advancer of FIG. 21.

FIG. 23 is a right side plan view of the guide wire advancer of FIG. 21.

FIG. 24 is a bottom left perspective view of the guide wire advancer of FIG. 21.

FIG. 25 is a front right perspective view of the guide wire advancer of FIG. 21.

FIG. 26 is a right rear perspective view of the guide wire advancer of FIG. 21 with the right cover partially transparent to show an example arrangement of internal drive components.

FIG. 27 is a right rear exploded perspective view of the guide wire advancer of FIG. 21.

FIG. 28 is a rear bird's eye exploded perspective view of the guide wire advancer of FIG. 21.

FIG. 29 is a left rear perspective view of internal components of the guide wire advancer of FIG. 21 shown with the housing removed.

FIG. 30A is a right side plan view of the guide wire advancer of FIG. 21 shown with the right housing removed to expose an example arrangement of internal components along with schematic representations pertaining to example operation of the actuator and automatic drive lock of the example configuration of FIG. 21, which is shown without a guide wire arranged therewith.

FIG. 30B is a close view of a portion of the actuator and automatic drive lock as identified in FIG. 30A along with the schematic representations of FIG. 30B.

FIG. 31 is another right side plan view of the guide wire advancer of FIG. 21 that is similar to FIG. 30B without including the schematic representations pertaining to the actuator and automatic drive lock, which includes schematic representations pertaining to routing and advancement of a guide wire along with identifying environmental information for close views (View G & View H) shown in more detail in FIGS. 32,34 and 35.

FIG. 32 is a close view of a tip portion of the guide wire advancer of FIG. 21 as identified in FIG. 31 (View G) showing a schematic representation for coupling with the example medical device shown configured as a suture placement device.

FIG. 33 is an upper right perspective of the guide wire advancer of FIG. 21 shown with the right cover removed providing a view of the nip portion of the drive mechanism.

FIG. 34 is a close side view of the nip portion of the drive mechanism for the guide wire advancer of FIG. 21 as identified in FIG. 31 (View H).

FIG. 35 is a schematic representation of the close view of the nip portion of the drive mechanism shown in FIG. 34, which illustrates additional options and features pertaining to the nip and drive components.

FIG. 36 is a left side plan view of the guide wire advancer of FIG. 21 with the left cover shown as partially transparent to expose the example arrangement of drive components and schematically represent aspects and features pertaining to the transmission switch feature including example movements of the float gear as indicated in subsequent Views I & J shown in FIGS. 37 and 38.

FIG. 37A shows View I identified in FIG. 36 and schematically represents additional potential options pertaining to automatic disengagement of the automatic drive in response to manual drive operations including movement aspects of the float gear and related transmission switch options.

FIG. 37B shows View J identified in FIG. 36 and schematically represents additional potential options pertaining to automatic engagement of the automatic drive in response to actuation of the actuator to enable automatic drive operations selected by the user including movement aspects of the float gear and transmission switch options and features.

FIG. 38A is a left side plan view of an example arrangement of an elongate device advancer in accordance with aspects and features of inventive concepts described herein, in which the example elongate device advancer is schematically depicted in an arrangement with the example suture placement device of FIG. 3D.

FIG. 38B is a proximal (rear) left side perspective view of the elongate device advancer of FIG.

FIG. 38C is another proximal (rear) left side perspective view of the elongate device advancer of FIG. 38A depicting removability of the stylet tip for replacing with a different tip arrangement.

FIG. 39A is a distal (front) left side perspective view of the elongate device advancer of FIG. 38A depicted in a usage arrangement held and controlled in a single hand of a user, and FIG. 39B is a top view thereof.

FIGS. 39C and 39D schematically depict a shape and corresponding contours from a plan view of the palm for an adult female hand (FIG. 39C) and for an adult male hand (FIG. 39D) along with schematically representing an orientation, grip, and usage location and orientation for the advancer of FIG. 398A within the example open palm for the male hand (FIG. 39D).

FIG. 39E schematically depicts a plan view representation of a palm of another representation of an adult hand along with showing corresponding anatomical medical labels, which are provided for clarity purposes when describing location and orientation of example advancers herein with respect to a user's hand.

FIG. 40A is a left side plan view of the elongate device advancer of FIG. 38A schematically depicting example curvatures of edge regions of the advancer.

FIG. 40B is a left side plan view of the elongate device advancer of FIG. 38A schematically depicting example angular arrangements and orientations between edge regions of the advancer.

FIG. 40C schematically depicts a plan view representation of a palm of another representation of an adult hand along with showing corresponding anatomical medical labels and the advancer of FIG. 40A shown therein, which are provided for clarity purposes when describing location and orientation of example advancers herein with respect to a user's hand.

FIG. 41A is a distal (front) left perspective view of the elongate device advancer of FIG. 38A.

FIG. 41B is a right plan view of the elongate device advancer of FIG. 38A.

FIG. 41C is proximal (rear) top left perspective view of the elongate device advancer of FIG. 38A.

FIG. 42A is a distal (front) right perspective view of the elongate device advancer of FIG. 38A.

FIG. 42B is a distal (front) view of the elongate device advancer of FIG. 38A, and FIG. 42C is a proximal (rear) view thereof.

FIGS. 43 and 44 are top plan views of the elongate device advancer of FIG. 38A showing internal components of a manual driver thereof contained within an advancer housing thereof (FIG. 43), and FIG. 44 is a top plan view of the internal components of the manual driver shown removed from the advancer housing.

FIG. 45 is right rear perspective view of a schematic representation of another elongate device advancer in accordance with aspects, features and inventive concepts described herein shown with the housing partially transparent to expose an example arrangement of internal components.

FIG. 46 is a right perspective view of drive rollers of the internal components for the elongate device advancer of FIG. 45.

FIG. 47 is a right rear exploded perspective view of the elongate device advancer of FIG. 45.

FIG. 48 is a top right exploded perspective view of the elongate device advancer of FIG. 45.

FIG. 49 is a perspective view of a roller assembly of the elongate device advancer of FIG. 45.

FIG. 50 is a cross-sectional perspective view of the roller assembly of FIG. 49 according to line A-A of FIG. 49.

FIG. 51A is a close view of a cross-section of the double seal O-ring denoted as View B in FIG. 50.

FIG. 51B is a cross-sectional view of an optional X-ring seal shown as an alternative for the double seal O-ring.

FIG. 52 is a right plan view of the elongate device advancer of FIG. 45 shown with the right housing partially transparent to expose an example arrangement of internal components along with example routing of an elongate device via an internal channel.

FIG. 53 is a right plan view of the elongate device advancer of FIG. 45 shown with the right housing removed to expose an example arrangement of internal components along with schematic representations pertaining to example operation of the drive rollers and outer roller assemblies of the example configuration of FIG. 45, which is shown with an elongate device arranged therewith.

FIG. 54 is a close view the stylet tip of the elongate device advancer of FIG. 45 for the region identified showing a schematic representation for coupling with the example medical device shown configured as the suture placement device of FIG. 3D.

FIG. 55 shows View C identified in FIG. 53, which is a close view of an elongate device being advanced through by the outer roller assemblies of the elongate device advancer of FIG. 45.

FIG. 56A is a top left perspective view of the elongate device advancer of FIG. 45 proximate the nip shown with the left housing removed for exposing the nip and interactions of the elongate device with the nip.

FIG. 56B is a close perspective view of the nip the advancer of FIG. 45 viewed as noted in FIG. 56A for View E, which is a close view from a top perspective generally corresponding with the orthographic side view of FIG. 53 and the close view of the nip of FIG. 55, which shows a portion of an elongate device that extends from a first, entry end of the pathway to an entry side of the nip without portions of the elongate device extending past the nip.

FIG. 57A shows a portion of the advancer of FIGS. 45 and 56B from a viewpoint proximate the nip as identified in FIG. 56B for region G, which is a Close View (G) of both front and rear double O-seal rings of the elongate device advancer of FIG. 45 proximate the nip and shown without having an elongate device within the pathway.

FIG. 57B shows a portion of the advancer of FIGS. 38A and 56B from a viewpoint proximate the nip as identified in FIG. 56B for region H, which is generally the same as FIG. 57A except with an elongate device shown disposed along the pathway and within the nip.

FIG. 58A is a rear perspective view of a lateral cross-section of the advancer of FIG. 45 at a location along the pathway as indicated in FIG. 56A for Views J & K, whereas FIG. 58B shows a plan view of the lateral cross-section as View K.

FIG. 59A is a rear perspective view of a lateral cross-section of the advancer of FIG. 45 at a different location along the pathway as indicated in FIG. 56B for Views L & M, whereas FIG. 59B shows a plan view of the lateral cross-section as View M.

FIG. 60A is a left plan view depicting another example elongate device advancer according to aspects and features described herein including for an advancer configured to translate a catheter, which is shown with the left housing removed for clarity.

FIG. 60B is a close cross-sectional view of the nip at an entry into the nip of the advancer of FIG. 30A as indicated in FIG. 30A for View N, which shows portions of O-seal rings for the first and second drive rollers during engagement of a catheter or other tube as the elongate device.

FIG. 60C is a schematic right-side plan view of a further example elongate device advancer according to aspects and features described herein, which shows an example conceptual arrangement of an advancer having an adjustment mechanism attached to the housing and the second drive roller for permitting adjustment of the advancer as appropriate for different types and properties of elongate devices.

FIG. 61 is a left side perspective view of a further example elongate device advancer according to aspects and features described herein, which schematically depicts a twist lock incorporated into a threaded introducer tip attachment that can be over-twisted to selectively engage a lock at the tip for restraining advancement and/or rotation of the elongate medical device routed through the advancer.

FIGS. 62A and 62B are top (62A) and bottom (62B) plan views of yet another example elongate device advancer according to aspects and features described herein, which schematically depicts a lateral lock incorporated into the advancer body at the distal region that can engage lateral portions of the elongate medical device within the pathway for selectively restraining advancement and/or rotation of the elongate medical device routed through the advancer.

FIG. 63 is a right side rear perspective view of a schematic representation for a handheld augmented grip connection elongate medical device advancer according to aspects and features described herein, which is similar in many respects to the example device 1310 shown in FIG. 11 without having features pertaining to combination manual and mechanical advancement. However, the particular example shown is schematic and merely depicted for illustration purposes.

FIGS. 64 and 65 are right side views of the example advancer of FIG. 63.

FIGS. 66 and 67 are right side views of another schematic representation of an example advancer similar to the example of FIG. 63 according to inventive aspects and features described herein.

FIGS. 68 and 69 are right side views of a further schematic representation of an example advancer similar to the example of FIG. 63 according to aspects and features described herein.

FIG. 70 is a left rear perspective view of an additional schematic representation of an example advancer according to inventive aspects and features described herein.

FIG. 71 is a top left exploded perspective view of the advancer of FIG. 70.

FIG. 72 is a left perspective view of the advancer of FIG. 70 shown with the left cover removed,

FIG. 73 is a left plan view of the advancer of FIG. 70 shown with portions of the internal components partially viewable for illustration purposes.

FIG. 74 is a left plan view of the advancer of FIG. 70 shown with the left cover removed.

FIGS. 75A and 75B are close views of a center portion of the left plan view of FIG. 74 shown in both the augmented and non-augmented conditions.

FIGS. 76A and 76B are top views of the inner portions of the advancer depicted in FIGS. 75A and 758.

FIG. 77 is a schematic representation of a method for advancing an elongate device and augmenting advancement of the same, such as with the example advancers of FIG. 63 or 70 in accordance with inventive aspects and features described herein.

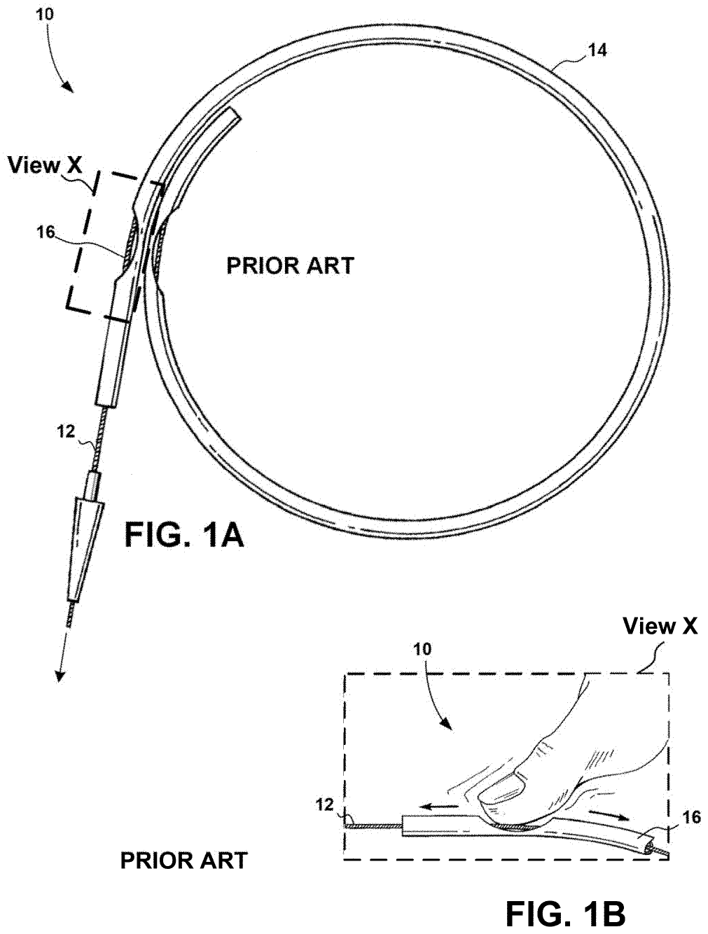

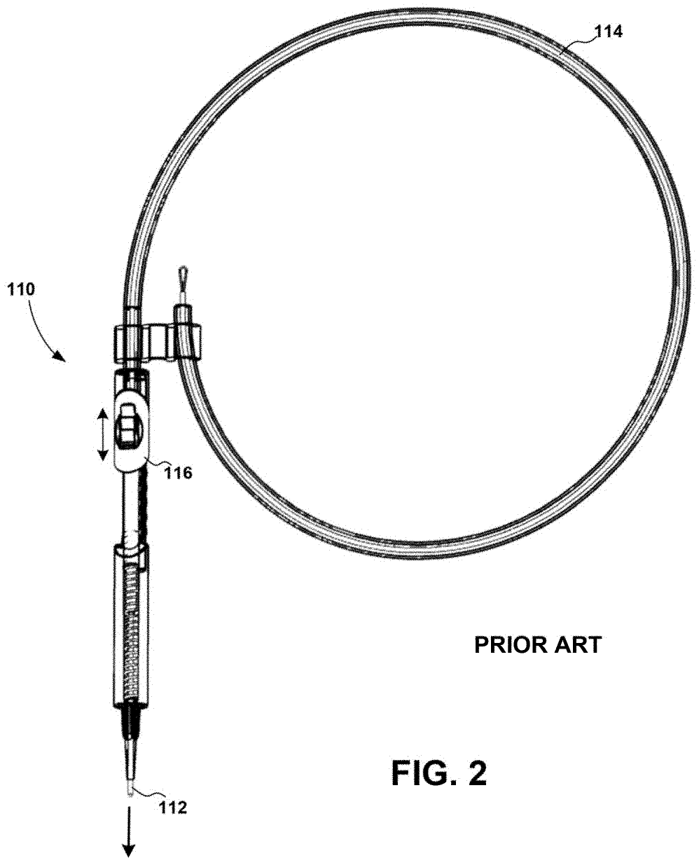

### DETAILED DESCRIPTION

The embodiments described herein can advantageously be used with a wide variety of surgical devices and procedures associated with minimally invasive surgery. In particular, the instruments described herein can be low-cost, disposable instruments that facilitate being used for only one procedure.

As used herein, the term “about” when used in connection with a referenced numeric indication means the referenced numeric indication plus or minus up to 10 percent of that referenced numeric indication. For example, the language “about 50” covers the range of 45 to 55. Similarly, the language “about 5” covers the range of 4.5 to 5.5.

As used herein, the term “target workspace” refers to anything within or pertaining to the endoscopic work cavity including the body of the patient, tissues and organs within the cavity, and tissue defining the cavity, and also to support structures for the MIS procedure including a cover and cannula supports, instruments and related attachments or medical implements including needles, suture materials, implants, meshes, etc. As used herein, the term “target tissue” refers to any tissue or organ that interacts with the target workspace including tissues and organs of the patient, natural tissues and organs introduced to the target workspace including natural transplant tissues and organs, artificial tissues and organs including mechanical or electro-mechanical organs, and tissue and organ assist devices such as pacemakers, mesh material, artificial skin and the like.

As used herein, a surgical device or tool or clinical instrument refers to a medical instrument having contact surfaces that are configured to engage organs, tissues and/or portions of a surgical cavity or wound to thereby move, hold, lift, retain, suture or otherwise engage, interface or make contact with the target tissue and perform clinical functions as appropriate for the surgical environment. The term “flexible” in association with a part, such as a mechanical structure, component, or component assembly, should be broadly construed. In essence, the term means the part can be repeatedly bent and restored to an original shape without harm to the part. Certain flexible components can also be resilient. For example, a component (e.g., a flexure) is said to be resilient if possesses the ability to absorb energy when it is deformed elastically, and then release the stored energy upon unloading (i.e., returning to its original state). Many “rigid” objects have a slight inherent resilient “bendiness” due to material properties, although such objects are not considered “flexible” as the term is used herein.

A flexible part may have infinite degrees of freedom (DOF's). Flexibility is an extensive property of the object being described, and thus is dependent upon the material from which the object is formed as well as certain physical characteristics of the object (e.g., cross-sectional shape, length, boundary conditions, etc.). For example, the flexibility of an object can be increased or decreased by selectively including in the object a material having a desired modulus of elasticity, flexural modulus, and/or hardness. The modulus of elasticity is an intensive property of (i.e., is intrinsic to) the constituent material and describes an object's tendency to elastically (i.e., non-permanently) deform in response to an applied force. A material having a high modulus of elasticity will not deflect as much as a material having a low modulus of elasticity in the presence of an equally applied stress. Thus, the flexibility of the object can be decreased, for example, by introducing into the object and/or constructing the object of a material having a relatively high modulus of elasticity.

As used in this specification and the appended claims, the word “distal” refers to direction towards a work site, and the word “proximal” refers to a direction away from the work site. Thus, for example, the end of a tool that is closest to the target tissue would be the distal end of the tool, and the end opposite the distal end (i.e., the end manipulated by the user or coupled to an actuation shaft) would be the proximal end of the tool.

Further, specific words chosen to describe one or more embodiments and optional elements, or features are not intended to limit the invention. For example, spatially relative terms—such as “beneath”, “below”, “lower”, “above”, “upper”, “proximal”, “distal”, and the like—may be used to describe the relationship of one element or feature to another element or feature as illustrated in the figures. These spatially relative terms are intended to encompass different positions (i.e., translational placements) and orientations (i.e., rotational placements) of a device in use or operation in addition to the position and orientation shown in the figures. For example, if a device in the figures were turned over, elements described as “below”, or “beneath” other elements or features would then be “above” or “over” the other elements or features. Thus, the term “below” can encompass both positions and orientations of above and below. A device may be otherwise oriented (e.g., rotated 90 degrees or at other orientations) and the spatially relative descriptors used herein interpreted accordingly. Likewise, descriptions of movement along (translation) and around (rotation) various axes include various spatial device positions and orientations. The combination of a body's position and orientation define the body's pose.

Similarly, geometric terms, such as “parallel”, “perpendicular”, “round”, or “square”, are not intended to require absolute mathematical precision, unless the context indicates otherwise. Instead, such geometric terms allow for variations due to manufacturing or equivalent functions. For example, if an element is described as “round” or “generally round,” a component that is not precisely circular (e.g., one that is slightly oblong or is a many-sided polygon) is still encompassed by this description.

In addition, the singular forms “a”, “an”, and “the” are intended to include the plural forms as well, unless the context indicates otherwise. The terms “comprises”, “includes”, “has”, and the like specify the presence of stated features, steps, operations, elements, components, etc. but do not preclude the presence or addition of one or more other features, steps, operations, elements, components, or groups.

As used in this specification, the reverse clutch refers to an advancer mechanism that can increase or enhance a default or existing drive connection between a nip of the advancer and an elongate device driven by the nip via the arrangement and corresponding component connections of the advancer, such as through the default nip and roller interference fit provided by the advancer. Whereas a vehicle clutch in a default state maintains a drive connection between driver and driven parts until engaged by a user to reduce and disconnect a driver/driven connection, a reverse clutch refers to a mechanism that maintains a default driver/driven connection until engaged by a user to increase, enhance or reinforce the driver/driven connection.

Unless indicated otherwise, the terms apparatus, medical device, instrument, and variants thereof, can be interchangeably used.

### Combination Automatically-Manually Driven Advancers

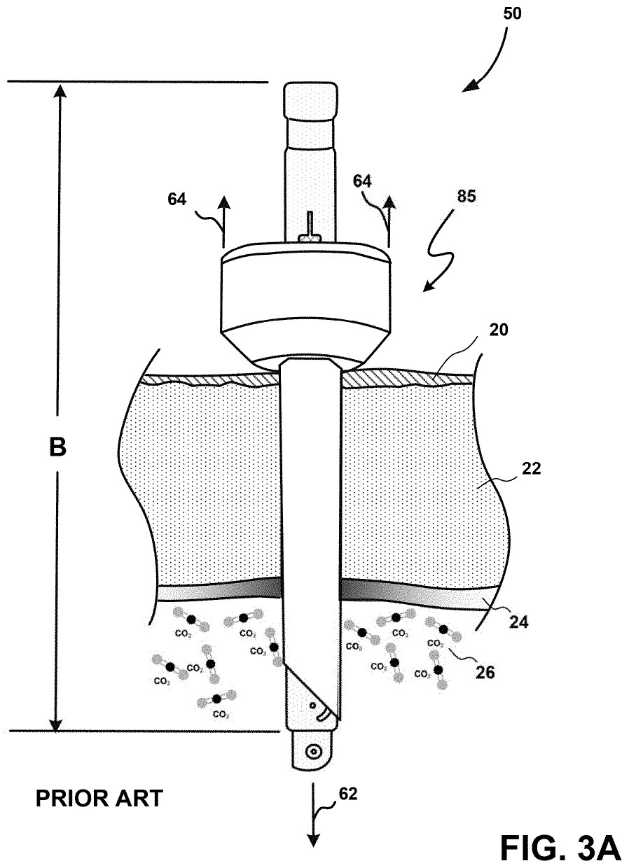

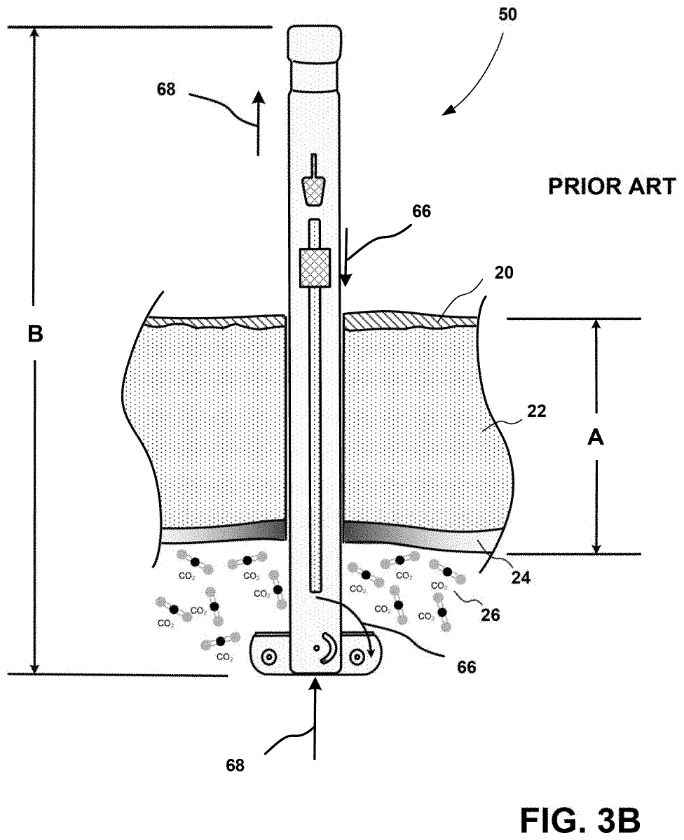

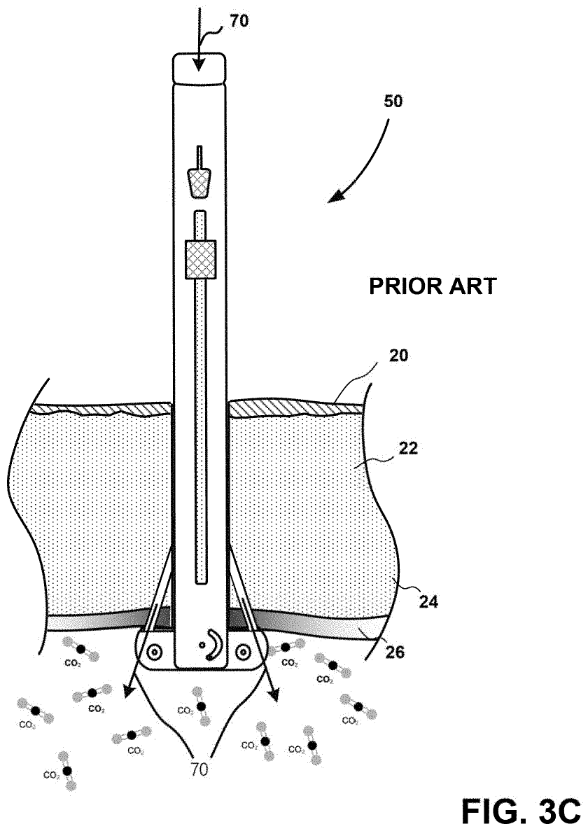

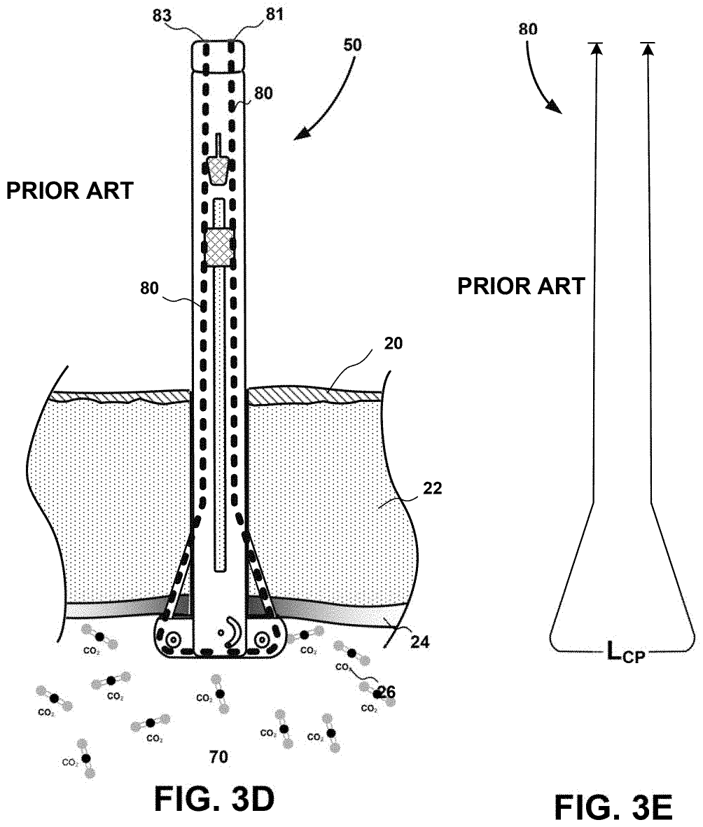

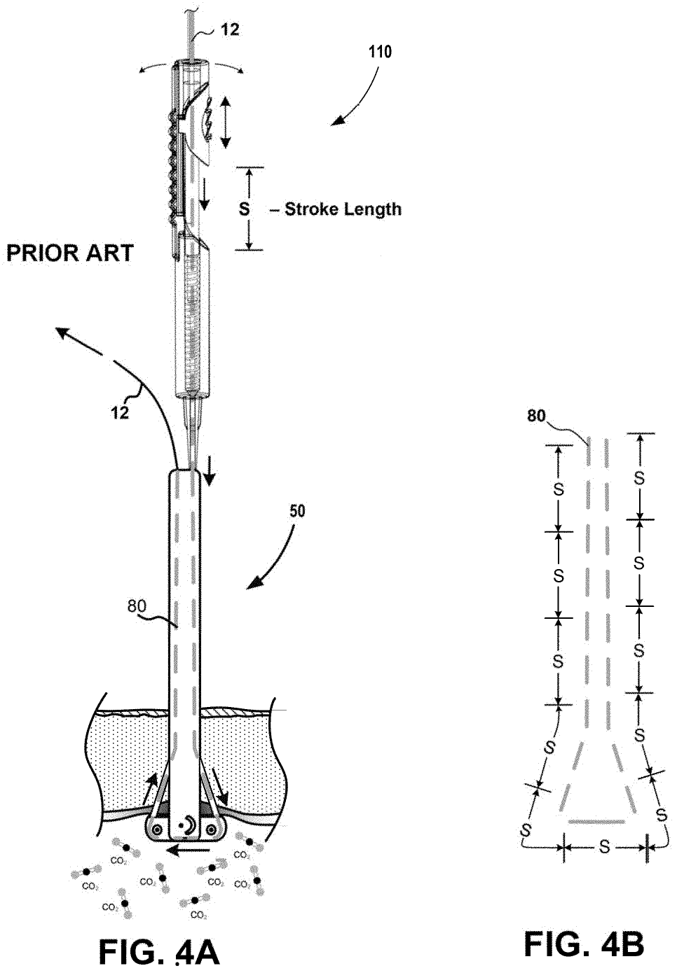

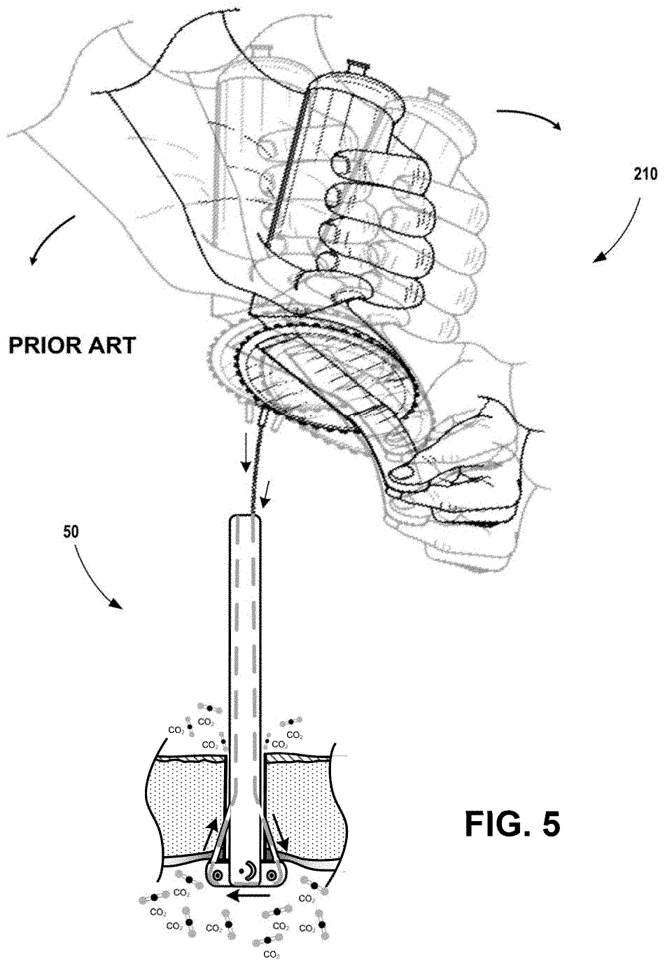

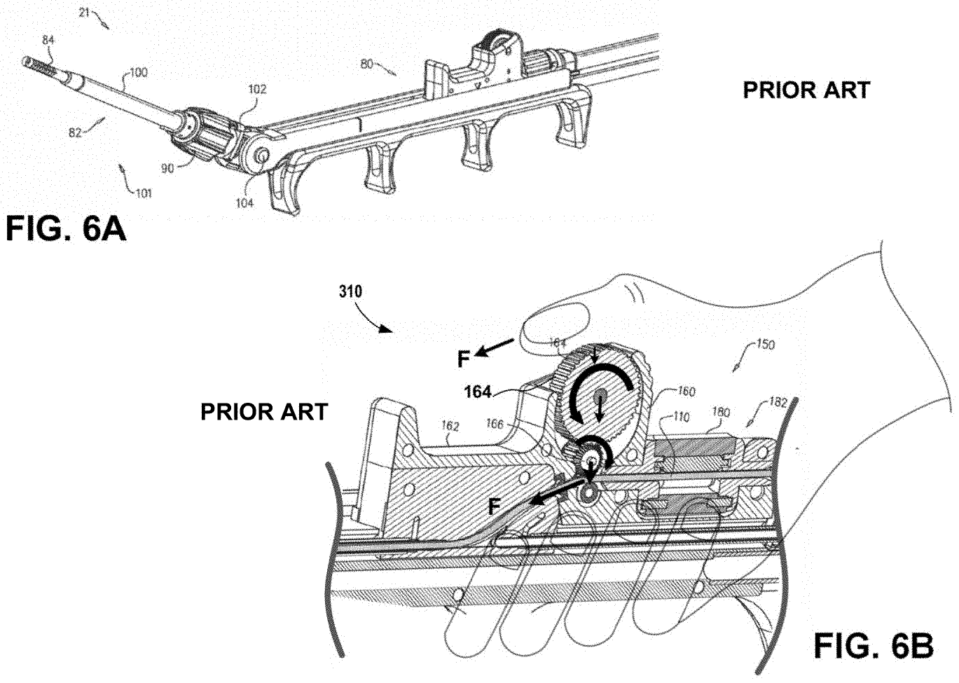

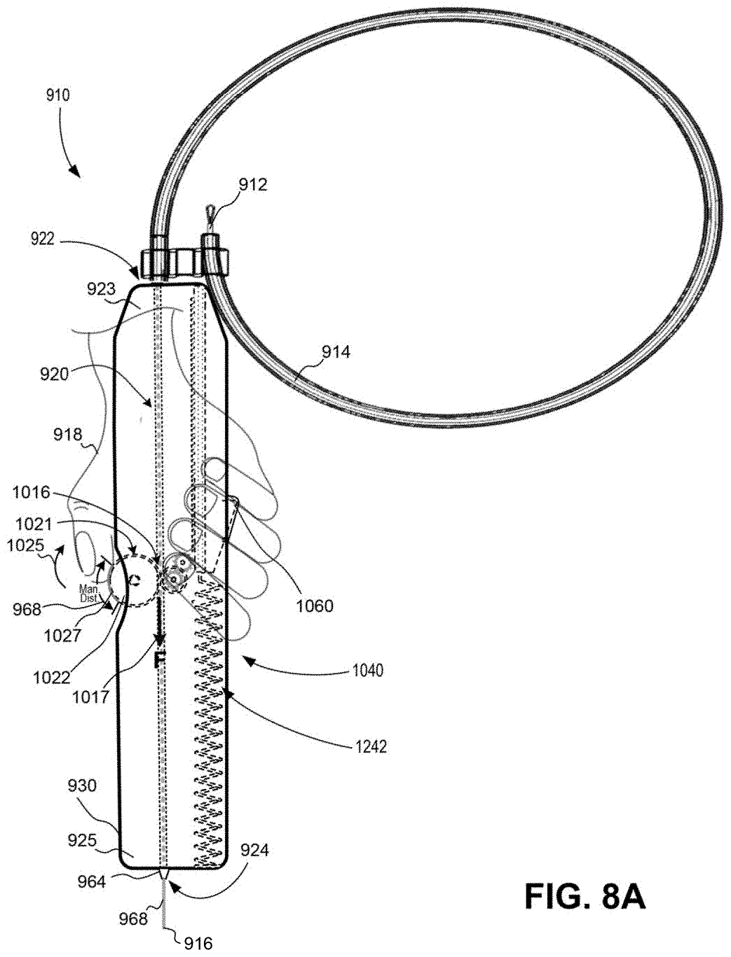

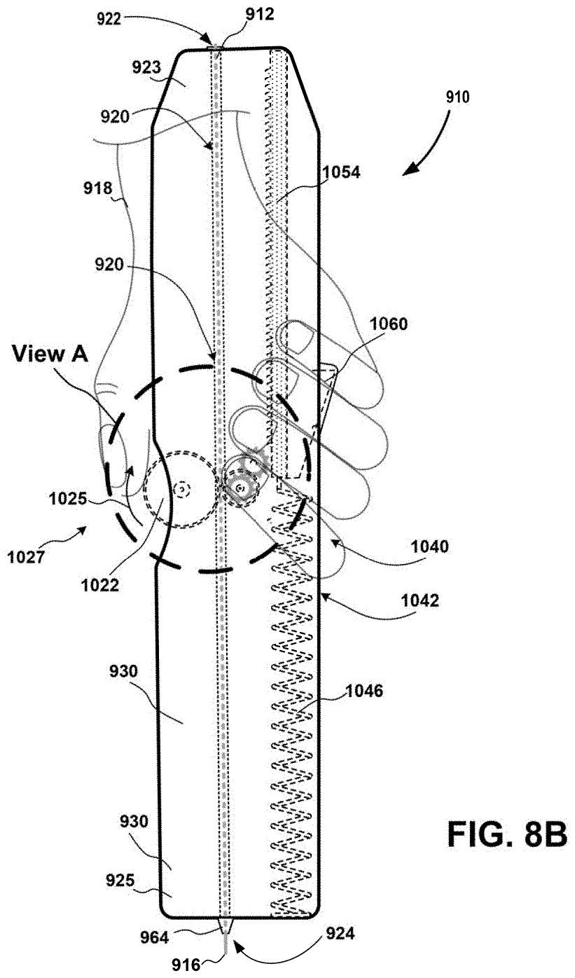

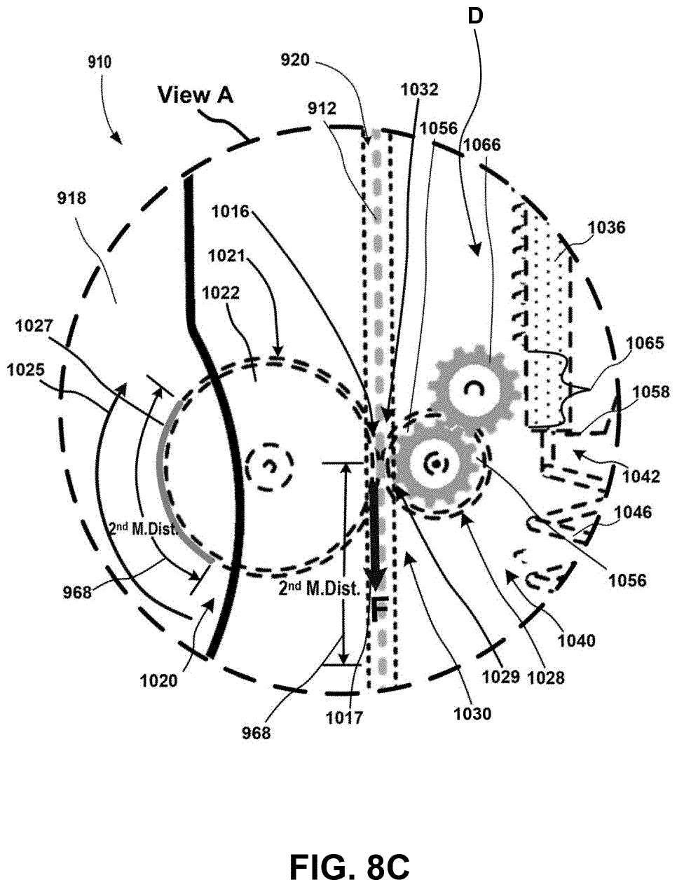

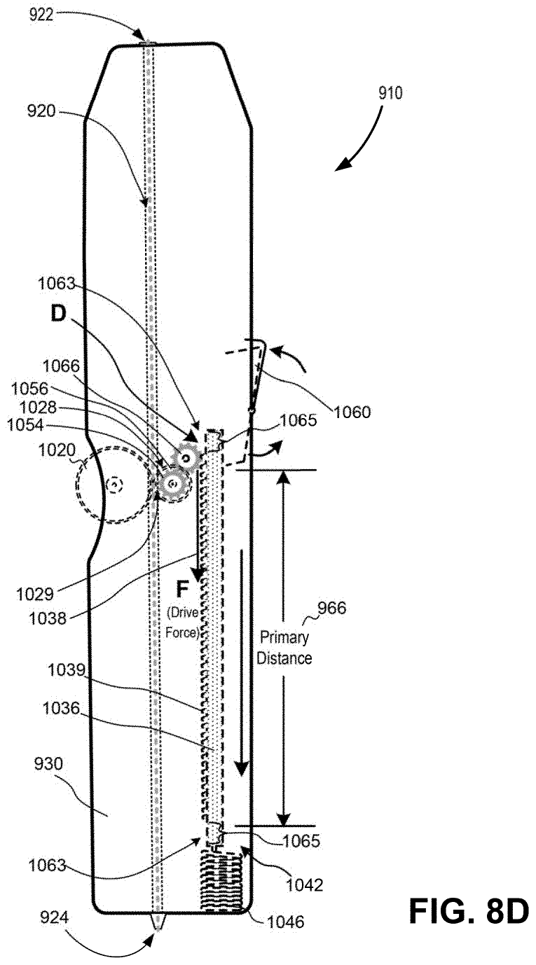

Referring now to FIGS. 8A-8D, a combination automatically-manually driven guide wire advancer 910 is shown for advancing a guide wire an automated primary distance 966 and a secondary manual distance 968 that is less than the primary distance, which as shown in FIG. 8A can be coupled with a guide wire/sheath coil 914 in a usage arrangement with the guide wire 912 of the coil. Although not shown, it is understood that the combination guide wire advancer 910 can be operatively configured with a surgical instrument including the suture placement device 50 discussed above along with FIGS. 3A-3E while disposed within a port and prepared for suture placement procedures. The combination guide wire advancer 910 is configured to be held within the single hand 918 of a user and easily controlled to perform guide wire advancement actions to support a surgical device, such as the suture placement device 50 discussed above.

The combination guide wire advancer 910 is advantageously configured for the user to actuate an automatic drive and readily advance the guide wire a primary first distance 966 based on a powered drive force stored by the guide wire advancer including automatically advancing the guide wire through a pathway of a coupled surgical device being supported by the guide wire advancer, such as advancing the guide wire within the channel pathway formed within the suture placement device of FIGS. 3A-3E. The combination guide wire advancer 910 is configured for the user to readily actuate the automatic drive using the single handle gripping the guide wire advancer, such as via movement of one or more fingers to actuate the automatic drive as desired. Further, the combination guide wire advancer 910 is advantageously configured for the user to exert control movements as well as using the single handle gripping the guide wire advancer, such as via rolling thumb movements exerted on a thumbwheel conveniently placed proximate the user's thumb. As such, the user can readily grip the advancer and easily control advancement operations including both an automated primary advancement of the guide wire and manual, fine-tune secondary advancement of the guide wire as desired.

Referring now to FIGS. 8A and 8B, the combination automatically-manually driven guide wire advancer 910 generally includes an advancer body 930, an automated drive 1040, a manual drive 1020, and an integrated advancement driver 1030. The advancer body 930 defines a guide wire pathway 920 for advancing the guide wire 912 therethrough from a first end portion 923 to a second end portion 925 of the advancer body, in which the advancer body is configured to be held in a grip of a single hand 918 of a user and controllable by the single hand. The first end portion 923 is configured to couple with the guide wire 912 to receive a tip portion 916 of the guide wire therein through an entrance 922 of the pathway 920 disposed at the first end portion 923, and the guide wire 912 is configured to advance through the pathway and out an exit 924 of the pathway disposed at the second end portion 925. The exit 924 can include a stylet tip 964 or similar arrangement for coupling the guide wire advancer in a support arrangement with a surgical device, such as a suture placement device.

As shown in FIGS. 8B-8D, the automated drive 1040 is configured to drive guide wire advancement for the primary distance 966 (FIG. 8D) when actuated. The automated drive 1040 generally includes a power driver 1042, an automated drive roller 1056, and an actuator 1060. The power driver 1042 is configured to store potential energy for driving guide wire advancement for the primary distance 966 (FIG. 8D) and transmit a corresponding automated drive force 1038 when actuated. The automated drive roller 1056 is rotatably coupled with the advancer body 1030 and has a resilient surface 1028 rotatable with the automated drive roller. The resilient surface 1028 includes a drive portion 1029 extending into the guide wire pathway 920 and configured to drivingly engage the guide wire 912. The actuator 1060 is configured to be actuated by the single hand 918 to activate the power driver 1042 to transmit the automated drive force 1038 to the automated drive roller drive portion 1029.

The power driver 1042 generally includes a spring 1046 coupled with a translatable rack 1036. In the deployable, stored energy condition shown in FIG. 8B, the spring 1046 is extended. A releasable lock 1058 (FIG. 8C) retains the translatable rack 1036 and spring 1046 in the deployable, stored energy condition until released by an actuator 1060 (FIG. 8B), which is readily accessible for the user to actuate via the single hand 918 when holding the guide wire advancer 910. When the actuator 1060 is actuated, it moves releasable lock 1058 to release the translatable rack 1036, which enables the rack to translate responsive to a drive force 1038 exerted on it by spring 1046. Drive teeth 1039 disposed along rack 1036 engage corresponding teeth on a pinion or floater gear 1066, which transfers the linear drive force 1038 as a rotary force to drive gear 1054 and drive roller 1056 attached thereto. Drive roller 1056 engages the guide wire 912 as described below via the nip 1029 to transfer linear drive force 1038 to the guide wire and drive its advancement for the primary distance 966.

As best seen in the close view of FIG. 8C, the manual drive 1020 is configured to drive guide wire advancement for the secondary distance 968 based on a user-exerted manual control movement 1025. The manual drive generally includes a manual drive roller 1022 and a manual control 1027, such as a thumbwheel or similar manually controllable device. The manual drive roller 1022 is rotatably coupled with the advancer body and has an engagement surface 1021 that is rotatable with the manual drive roller. The engagement surface 1021 includes a drive portion 1016 extending into the guide wire pathway 920 and configured to drivingly engage the guide wire 912. The manual control 1027 is configured to receive the user-exerted manual control movement 1025 from the single hand 918 and transmit a corresponding manual drive force 1017 to the manual drive roller drive portion 1016 for imparting guide wire advancement for the second distance 968.

For the relatively simple configuration of guide wire advancer 910, the range of movement for the single hand of the user with respect to the manual control 1027 generally corresponds with the greatest secondary distance 968 available for manual advancement, and the manual control movements act as a direct drive for manually advancing the guide wire. However, other configurations can provide greater manual control options, such as geared connections between the user-accessible manual control and one or more drive components that exert advancement force on the guide wire. Further, one or more mechanical advantage options or switchable arrangements can optionally be provided to enable enhanced manual controls and selectable or configurable manual advancement settings.

The integrated advancement driver 1030 generally includes a cooperative feed roller nip 1032 configured to advance the guide wire in response to transmission of the automated drive force or the manual drive force to a corresponding one of the drive portions. In other words, the integrated advancement driver 1030 is configured as a multi-source advancement driver that can readily advance the guide wire according to the user's control movements including responsive to user-exerted manual control or manual drive force 1017, and in response to user-actuation of automatic advancement to provide powered drive force 1038. As such, the cooperative feed roller nip 1032 includes a drive roller from both the manual drive 1020 and the automated drive 1040. Further, the cooperative feed roller nip 1032 is defined between the drive portion 1016 of the manual drive roller 1022 that extends into the guide wire pathway, which is configured in an opposed arrangement with the drive portion 1029 of the automated drive roller 1056 that likewise extends into the guide wire pathway. The opposing drive portions can be configured to engage opposite side regions of the guide wire and cooperate as a pair of feed rollers for advancing the guide wire 912 along the pathway 920 toward the second end portion. Further, the opposing drive portions can each be configured to function as both a drive roller and a driven roller based on drive roller is acting as the operative drive providing drive force.

Referring now to FIGS. 8B-8D, the guide wire advancer can include a transmission switch 1063 configured for automatic movement between a first position D and a second position E, in which the first position D (FIG. 8C) de-couples the power driver from the automated drive roller when the manual control receives the user-exerted manual control movement, and the second position E (FIG. 8D) drivingly engages or connects the power driver to the powered drive roller 1056 when the actuator is actuated. The transmission switch 1063 in the example configuration for guide wire advancer 910 generally includes a floater gear 1066 and a pair of neutral portions 1065 that are each disposed at an opposite end portion of translatable rack 1036 extending beyond its drive teeth 1039. Floater gear 1066 acts as an intermediate gear between rack 1036 and powered drive gear 1054 as described above along with the power driver 1042 that engages rack 1036 with powered drive gear 1054 while in second position E during translation of the rack. The first position D includes one or more de-coupling positions with or without the power driver 1042 storing potential energy, such as before or after actuation of the power driver.

For instance, as shown in FIG. 8C, prior to actuation of the actuator 1060 (FIG. 8C), releasable lock 1058 retains rack 1036 such that the transmission switch 1063 is in the first position D de-coupled from engagement with power drive gear 1054 via disengagement from floater gear 1066. In particular, a distal one of the neutral portions 1065 disposed at end portion of rack 1036 is located proximate floater gear 1066, which de-couples the power driver 1042 from the automated or power drive roller 1056 due to non-engagement between the floater gear and the rack. As such, the user is free to exert manual control movements 1025 upon the manual control portion 1027 of thumbwheel 1022 and advance the guide wire 912 up to the second advancement distance one or more times as desired, such as for advancing the guide wire through the guide wire pathway 920, out of exit 924, and into entry port 81 of a suture placement device 50 described above along with FIGS. 3A-3D.

In addition, after actuation of the actuator 1060 completes, the transmission switch 1063 is again disposed in the first position D de-coupled from engagement with power drive gear 1054 via disengagement from floater gear 1066. In particular, as can best be seen in FIG. 8D, a proximal one of the neutral portions 1065 disposed at end portion of rack 1036 is located proximate floater gear 1066 upon completion of deployment of the power driver 1042, which de-couples the power driver from the automated or power drive roller 1056 due to non-engagement between the floater gear and the rack. As such, the user is again free to exert manual control movements 1025 upon the manual control portion 1027 of thumbwheel 1022 and advance the guide wire 912 up to the second advancement distance one or more times as desired, such as for advancing the guide wire through the remainder of channel pathway 80 of a suture placement device 50 described above along with FIGS. 3A-3D.

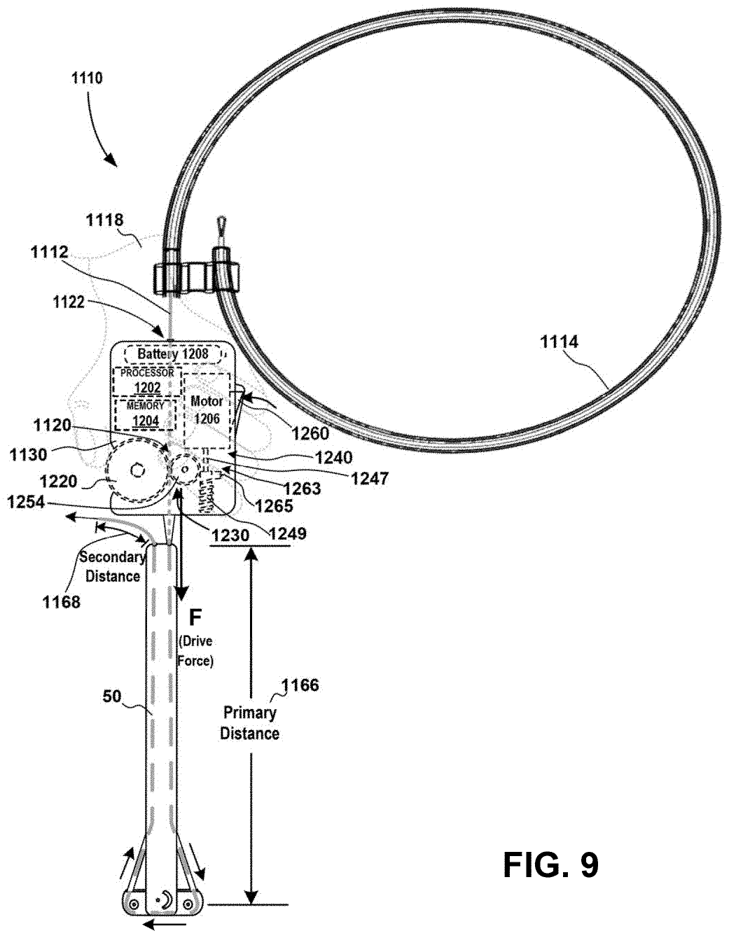

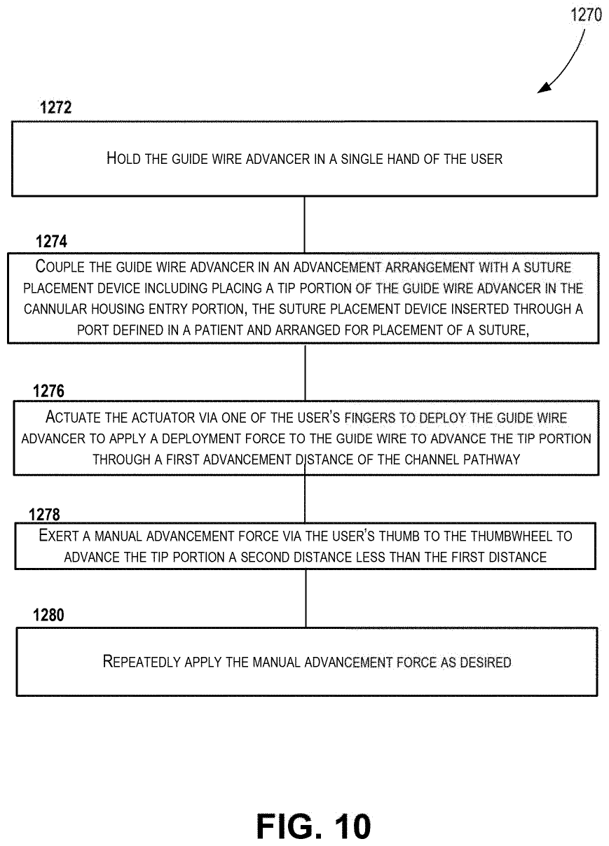

Referring now to FIG. 10 along with FIGS. 8A-8D and FIG. 9 discussed in greater detail below, a method 1270 for advancing a guide wire using guide wire advancer 910 coupled with suture placement device 50 discussed above is generally shown. The method 1270 generally includes step 1272 of holding the guide wire advancer 910 in a single hand 918 of the user, and step 1274 of coupling the guide wire advancer 910 in an advancement arrangement with a suture placement device 50 including placing a tip portion or stylet tip 964 of the guide wire advancer in the cannular housing entry portion proximate the entry port 81 of the suture placement device, which is inserted through a port defined in a patient and arranged for placement of a suture. The method can optionally include step 1275 (not shown) of advancing the guide wire one or more secondary advancement distances 968 less than an automated second advancement distance to advance the guide wire 912 through the entry port 81.

Method 1270 further includes the step 1276 of actuating via one of the user's fingers of the hand 918 the actuator 1260 to deploy a deployment drive force 1038 to the guide wire 912 to advance a tip portion 916 through a first advancement or primary distance 966 of the suture placement device channel pathway 80. The method 1270 continues with step 1278 of exerting a manual advancement or drive force 917 via the user's thumb of the single hand 918 to advance the tip portion a secondary distance 968 that is less than the primary distance 966, and optionally step 1280 of repeatedly applying the manual advancement force 917 as desired, such as to advance the tip portion of the guide wire through and out of exit port 83 of the suture placement device 50.

Referring now to FIG. 9, a combination automatically-manually driven guide wire advancer 1110 is shown for advancing a guide wire an automated primary distance 1166 and a secondary manual distance 1168 that is less than the primary distance, which as shown in FIG. 9 can be coupled with a guide wire/sheath coil 1114 in a usage arrangement with the guide wire 1112 of the coil. Guide wire advancer 1110 generally includes the same or similar aspects and features as guide wire advancer 910 except as shown or discussed herein. Accordingly, like numbers general refer to like features. Similar to guide wire advancer 910, it is understood that the combination guide wire advancer 1110 can be operatively configured with a surgical instrument including the suture placement device 50 discussed above along with FIGS. 3A-3E and as shown in FIG. 9, which can be while suture placement device 50 is disposed within a port through a patient's body (not shown) and prepared for suture placement procedures. The combination guide wire advancer 1110 is likewise configured to be held within the single hand 1118 of a user and easily controlled to perform guide wire advancement actions to support a surgical device, such as the suture placement device 50 discussed above.

Similar to guide wire advancer 910, the combination guide wire advancer 1110 is advantageously configured for the user to actuate an automatic drive and readily advance the guide wire a primary first distance 1166 based on a powered drive force stored by the guide wire advancer including automatically advancing the guide wire through a pathway of a coupled surgical device being supported by the guide wire advancer, such as advancing the guide wire within the channel pathway formed within the suture placement device 50. The combination guide wire advancer 1110 is configured for the user to readily actuate the automatic drive using the single handle gripping the guide wire advancer, such as via movement of one or more fingers to actuate the automatic drive as desired. Further, the combination guide wire advancer 1110 is advantageously configured for the user to exert control movements as well as using the single handle gripping the guide wire advancer, such as via rolling thumb movements exerted on a manual control conveniently placed proximate the user's thumb. As such, the user can readily grip the advancer and easily control advancement operations including both an automated primary advancement of the guide wire and manual, fine-tune secondary advancement of the guide wire as desired.

The combination automatically-manually driven guide wire advancer 1110 generally includes an advancer body 1130, an automated drive 1240, a manual drive 1220, and an integrated advancement driver 1230, which can be configured similar to corresponding aspects and features described along with guide wire advancer 910 with the exception of the automated drive 1240. Further, guide wire advancer 1110 can include a similar transmission switch 1263 to transmission switch 963 including a neutral portion 1265 that is configured to dis-engage the automated drive 1240 from a drive roller of the integrated advancement driver 1230 except during operation of the automated drive 1240 to advance the guide wire 1112 for the primary distance 1166.

However, the automated drive 1240 differs from automated drive 1040 of guide wire advancer 910 in that automated drive 1240 is configured as an electrically powered, motor-driven automated drive. Automated drive 1040 generally includes an electric motor 1206 that is electrically connected to a power supply 1208, such as an arrangement of capacitors or a battery 1208, a logic control unit 1202, such as a processor 1202, and memory 1204 that can be configured separately or as part of logic control unit/processor 1202, such as firmware or read-only memory (ROM). The electric motor further includes a drive shaft 1247 drivingly connected with the motor 1206, and a worm gear 1249 attached to the drive shaft. It should be understood that the operating environment and the various components of the automated drive 1240 have been greatly simplified for purposes of discussion. Accordingly, additional or alternative components can be made available without departing from the embodiments described herein.

The battery 1208 provides power to motor 1206, which operates to provide a drive force when actuated to advance the guide wire 1212 the primary distance 1166. The use of a motor 1206 and power supply can provide several advantages including allowing the user to quickly and easily repeatedly advance the guide wire for the primary distance as needed for use with a particular procedure and surgical device, such as suture placement device 50, or for usage with additional suture placement devices for closing multiple ports near the end of a surgical operation or for usage with other surgical devices during a surgical operation. Further, usage of an electrically powered motor can permit guide wire advancer 1210 to be configured as a smaller, more compact and ergonomic device compared with a mechanically powered device.

In addition, usage of an electric motor in combination with control logic and modifiable control options, such as an including a processor 1202 and memory 1204 configured to control operations of powered drive 1240 when actuated can provide a wide range of options, customizations, user preferences, procedure-specific parameters and the like to entered or applied as parameters for operations of the automated drive. For example, the guide wire advancer 1110 can permit customization for use with a variety of surgical devices, types of procedures, and various parameters of the same such as guide wire gauge, pushability, length of the channel pathway, etc., as well as for user preferences, such as options for using multiple primary distances of differing lengths. The processor 1202 and memory 1204 are in electrical communication with the battery 1208 and motor 1206. The memory stores computer-readable instructions, which are processed by the processor to control operations of the motor 1206 and/or supply operating power to the motor from the battery 1208, such as controlling the current and/or voltage applied to the motor upon actuation of the actuator 1260 and the duration of power supply to provide for advancing the guide wire 1112 the primary distance 1166.

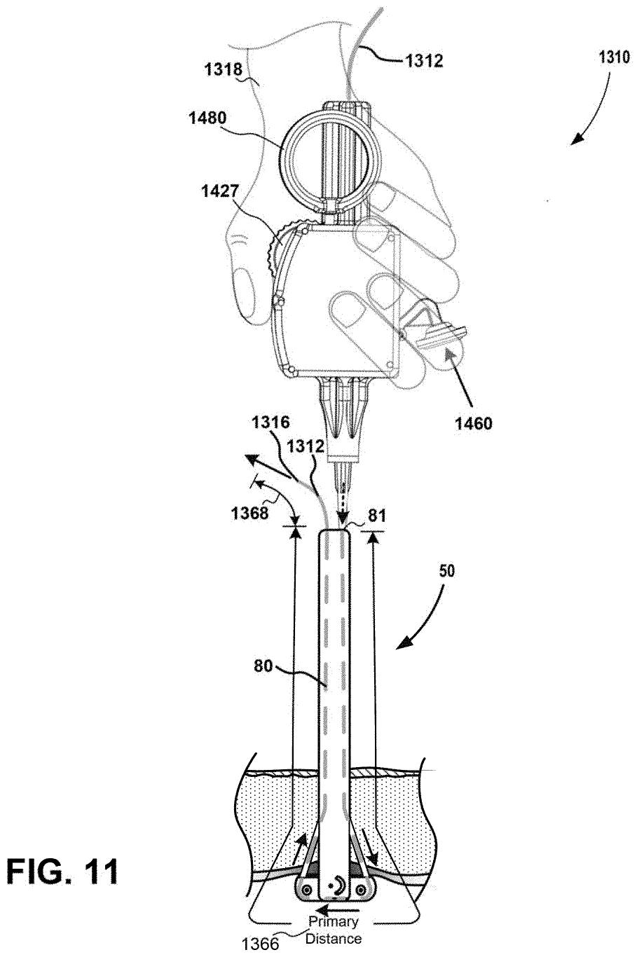

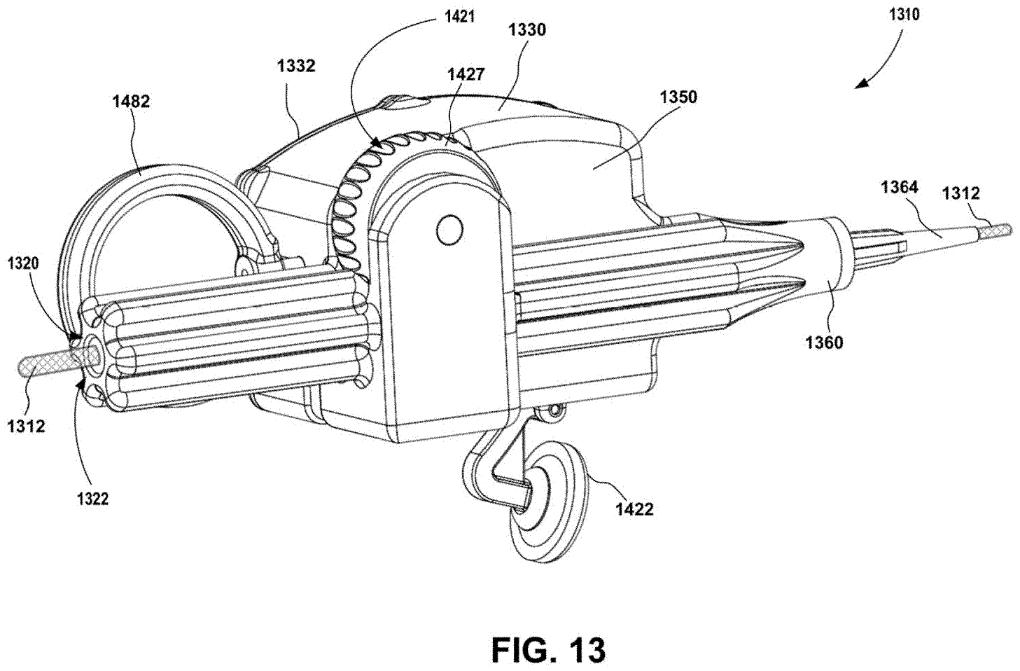

Referring now to FIGS. 11-20 and in particular to FIG. 11, a combination automatically-manually driven guide wire advancer 1310 is shown for advancing a guide wire an automated primary distance 1366 and a secondary manual distance 1368 that is less than the primary distance, which can be coupled with a guide wire/sheath coil (not shown) in a usage arrangement with the guide wire 1312 of the coil. It is understood that the combination guide wire advancer 1310 can be operatively configured with a surgical instrument including the suture placement device 50 discussed above along with FIGS. 3A-3E and shown in FIG. 11 while the surgical instrument is disposed within a port and prepared for suture placement procedures, as well as with other similar and non-similar types of surgical instruments (not shown). The combination guide wire advancer 1310 is configured to be held within the single hand 1318 of a user and easily controlled to perform guide wire advancement actions to support the surgical instrument, such as suture placement device 50 discussed.

The combination guide wire advancer 1310 includes many aspects and features that are the same as or similar to those discussed above with guide wire advancers 910 and 1110. Accordingly, like numbers refer to like features. However, guide wire advancer 1310 further includes aspects and features not discussed along with guide wire advancers 910 and 1110 or that differ therefrom, which are discussed below and/or shown in FIGS. 11-20. Combination guide wire advancer 1310 is advantageously configured for the user to actuate an automatic drive and readily advance the guide wire a primary first distance 1366 based on potential energy stored therein. Such a configuration can provide a powered drive force to automatically advance the guide wire through a pathway of a coupled surgical device being supported by the guide wire advancer, like advancing the guide wire within a channel pathway 80 formed within the suture placement device 50. The combination guide wire advancer 1310 is further configured as a compact, ergonomic, high-power advancement device arranged for the user to readily actuate the automatic drive using a single hand 1318 gripping the guide wire advancer. For example, as shown in FIG. 11, movement of one or more fingers of the gripping hand 1318 can readily actuate the automatic drive by depressing an actuator 1460. Further, the combination guide wire advancer 1310 is advantageously configured for the user to exert control movements via the same gripping hand 1318 while gripping the guide wire advancer, such as via rolling thumb movements exerted on a manual control 1427 conveniently placed proximate the user's thumb. As such, the user can readily grip the advancer and easily control advancement operations including both an automated primary advancement of the guide wire and manual, fine-tune secondary advancement of the guide wire.

Further, combination guide wire advancer 1310 includes a compact, high-power mechanical power source, which can readily be re-charged or re-set by pulling a pull tab to enable repeated powered advancements of the guide wire. Further, combination guide wire advancer 1310 is configured in a general straight-through advancement arrangement, which minimizes the amount of friction or resistance for advancing the guide wire related to the guide wire advancer along with efficiently transferring advancement forces from the advancer to the guide wire with minimal losses. Thus, maximal portions of applied powered and manual drive forces can be transferred to the guide wire 1312 for its advancement through the corresponding surgical device.

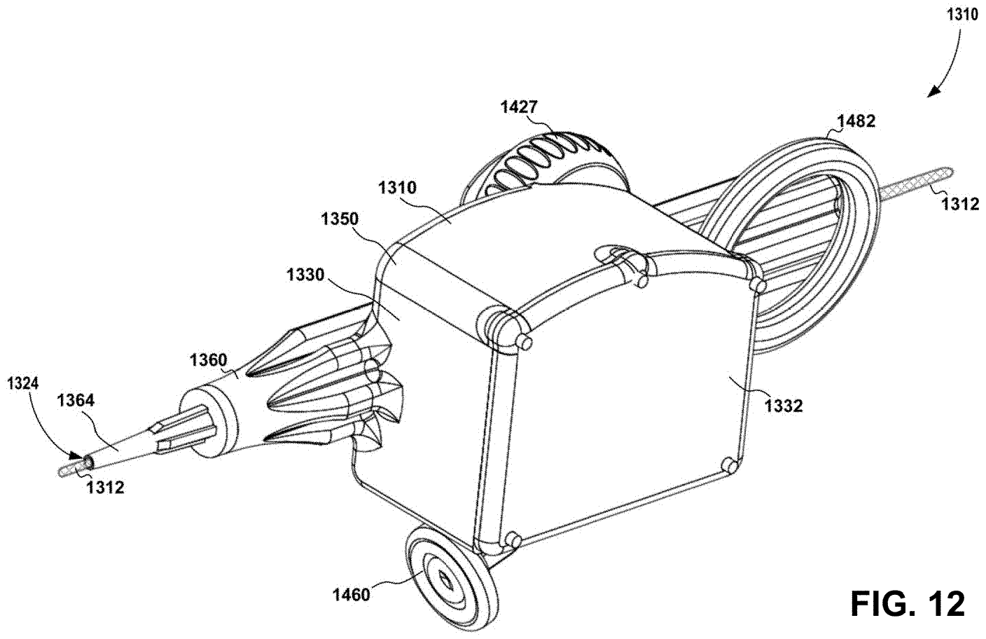

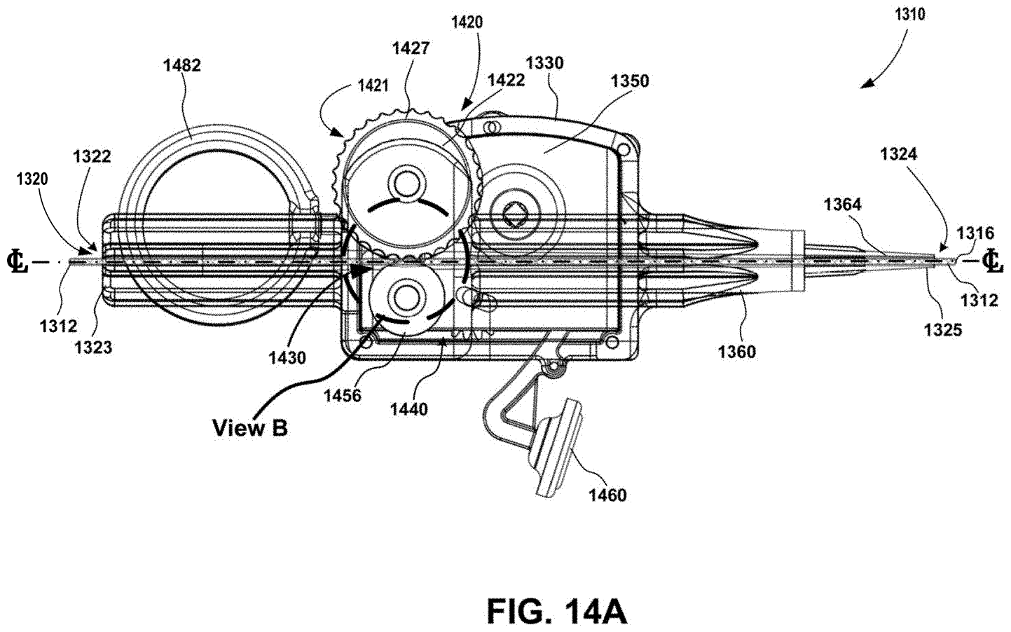

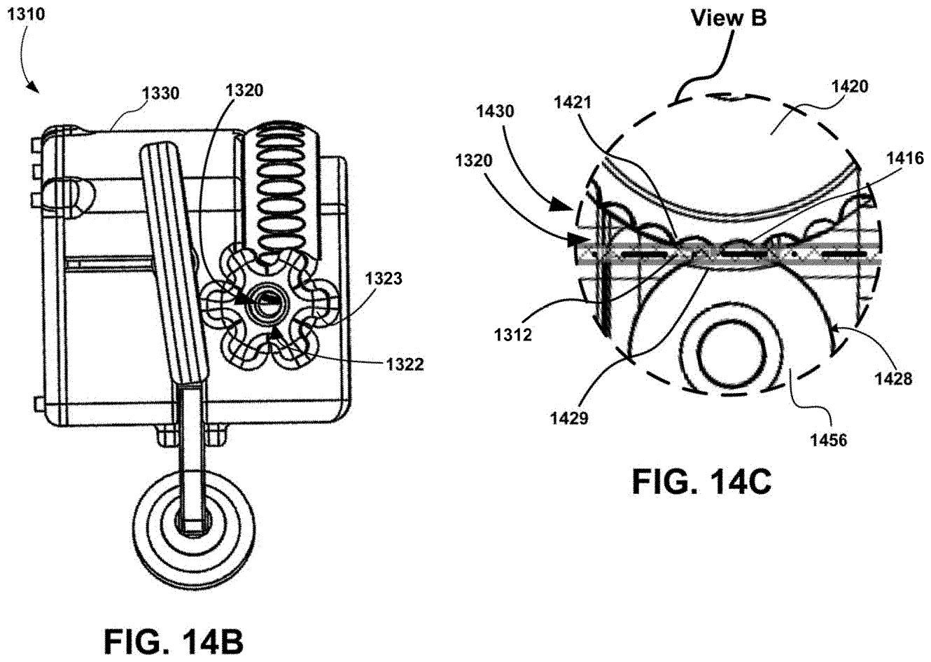

Referring now to FIGS. 12-15B, the combination automatically-manually driven guide wire advancer 1310 generally includes an advancer body 1330, an automated or power drive 1440, a manual drive 1420, and an integrated advancement driver 1430. The advancer body 1330 defines a guide wire pathway 1320 (FIG. 14A) for advancing the guide wire 1312 therethrough from a first end portion 1323 to a second end portion 1325 of the advancer body, in which the advancer body is configured to be held in a grip of a single hand 1318 of a user and controllable by the single hand. The first end portion 1323 is configured to couple with the guide wire 1312 to receive a tip portion 1316 of the guide wire therein through an entrance 1322 of the pathway 1320 disposed at the first end portion 1323, and the guide wire 1312 is configured to advance through the pathway and out an exit 1324 of the pathway disposed at the second end portion 1325. The exit 1324 can include a stylet tip 1364 or similar arrangement for coupling the guide wire advancer in a support arrangement with a surgical device, such as suture placement device 50.

The advancer body 1330 includes a left housing 1332, a right housing 1350, and an introducer or stylet 1360 integrally formed with the right housing 1350 on the right side of the advancer body 1330. The guide wire pathway 1320 is formed through the stylet 1360 portion of the body as a generally straight-line channel through the centerline ℄ of the stylet 1360. The guide wire pathway entrance 1322 (FIGS. 14A&B) is formed at the first end portion 1323 along the centerline ℄ of the stylet and guide wire pathway 1320, and the exit 1324 is formed at the second end portion 1325 along the centerline ℄ of the stylet and guide wire pathway. The integrated advancement driver 1430 is disposed along the guide wire pathway 1320 (FIG. 14A) within the stylet 1360, which as discussed further below is configured to apply drive forces in the advancement direction aligned with the centerline ℄ of the guide wire pathway 1320. As such, the guide wire advancer 1310 is configured to efficiently push or ‘shoot’ the guide wire through a low-friction, straight path lacking bends or features providing resistance, out of the stylet tip 1354 and exit 1324, and directly into an entry port of the corresponding surgical device (e.g., entry port of suture placement device 50). The guide wire advancer 1310 is configured to do so while closely supporting the guide wire within the guide wire pathway 1320 to prevent the guide wire from bending, kinking or folding over on itself. As such, guide wire advancer 1310 provides high efficiency, low loss, advancement of the guide wire 1312 directly into and along a channel pathway 80 of a corresponding surgical device 50.

As best seen in FIG. 11, upon actuation of actuator 1460, the automated power drive 1440 is configured to drive guide wire advancement for the primary distance 1366, which can be configured to correspond with substantially the entire length of the channel pathway 80 through the corresponding surgical device 50. The manual drive 1420 is configured to drive guide wire advancement for the secondary distance 1368 based on a user-exerted manual control movement on control 1427 that is configured as an exposed portion of a thumbwheel 1422. The exposed control 1427 of thumbwheel 1422 is configured to receive a user-exerted manual control movement (not shown) from the single hand 1318, such as rolling thumbwheel 1422, to transmit a corresponding manual drive force to the guide wire 1312 via the manual drive 1420 as discussed further below along for advancement of the guide wire 1312 the second distance 1368. One or more user-exerted manual control movements (not shown) can be applied by rolling the exposed control portion 1427 of thumbwheel 1422 repeatedly, which can beneficially be applied after actuation of the power drive to advance the guide wire the primary distance 1366, such as for manually advancing the guide wire tip portion 1316 to extend a desired length out of the surgical device. Manual control movements (not shown) can also be applied prior to actuation of the power drive, such as for manually advancing the guide wire tip portion 1316 through the length of the guide wire pathway and extend from the exit 1324 a desired length, such as for ensuring proper alignment and mating with a surgical device including extending into guide wire entry port 81 of suture placement device 50.

Thus, the combination of manual drive 1420 and automated power drive 1440 provides the user with significant control for advancing the guide wire as desired when aligning and mating the guide wire advancer 1310 with a surgical device, for quick and efficient deployment of the guide wire to advance through the pathway of the surgical device, and for arranging the guide wire for surgical procedures after deployment through the surgical device pathway. In addition, guide wire advancer 1310 is configured to efficiently advance the guide wire through and out of the advancer with minimal drive losses or opportunity for the guide wire to bend, kink or fold over. These advantages are provided in compact device configured to be held and controlled via the single hand 1318 of a user, which can further be re-set or re-charged as discussed further below to provide multiple actuations of the power drive as appropriate for use, for instance, with surgical devices having extended channel pathways, and for multiple uses with the same or similar surgical devices including multiple suture placement devices for performing multiple port closure procedures. Many of these advantages and other benefits provided by guide wire advancer 1310 are related to the advantageous integration and combination of drives within the guide wire advancer including manual drive 1420, automated power drive 1440, and integrated combination drive 1430.

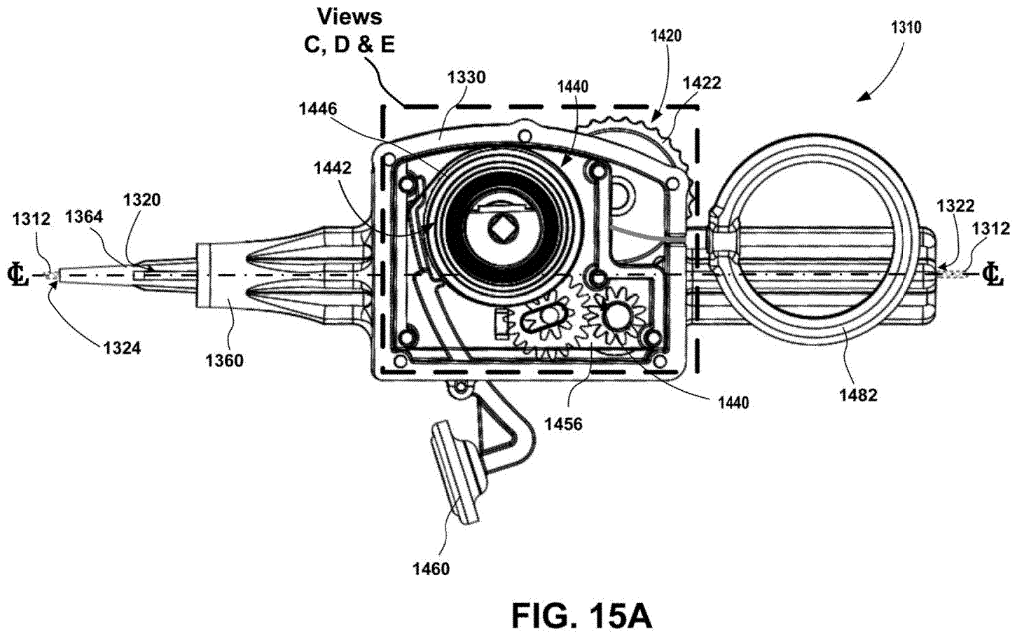

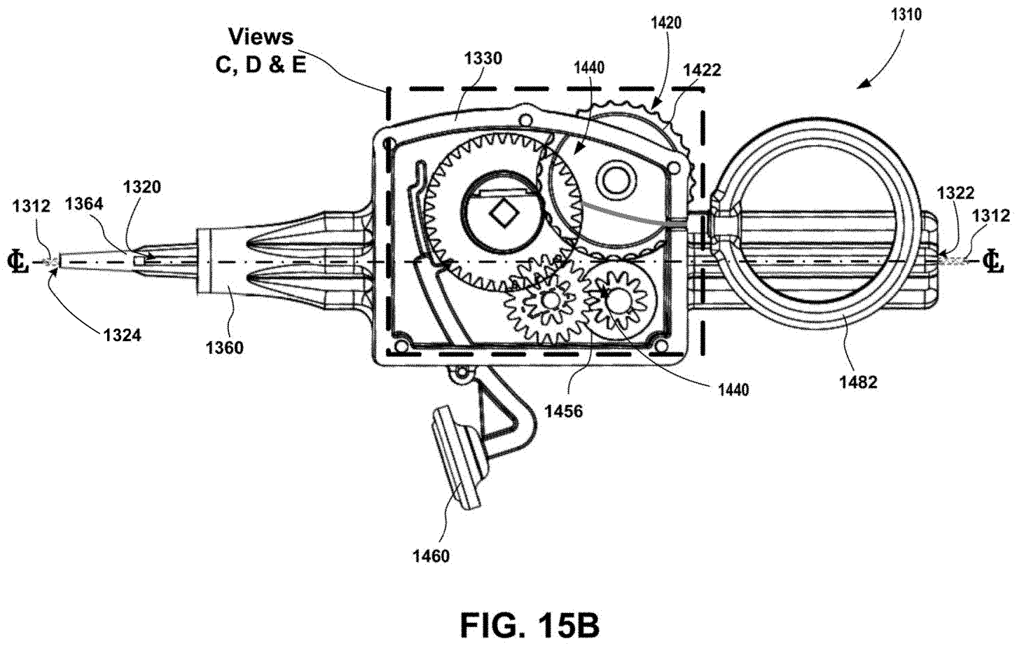

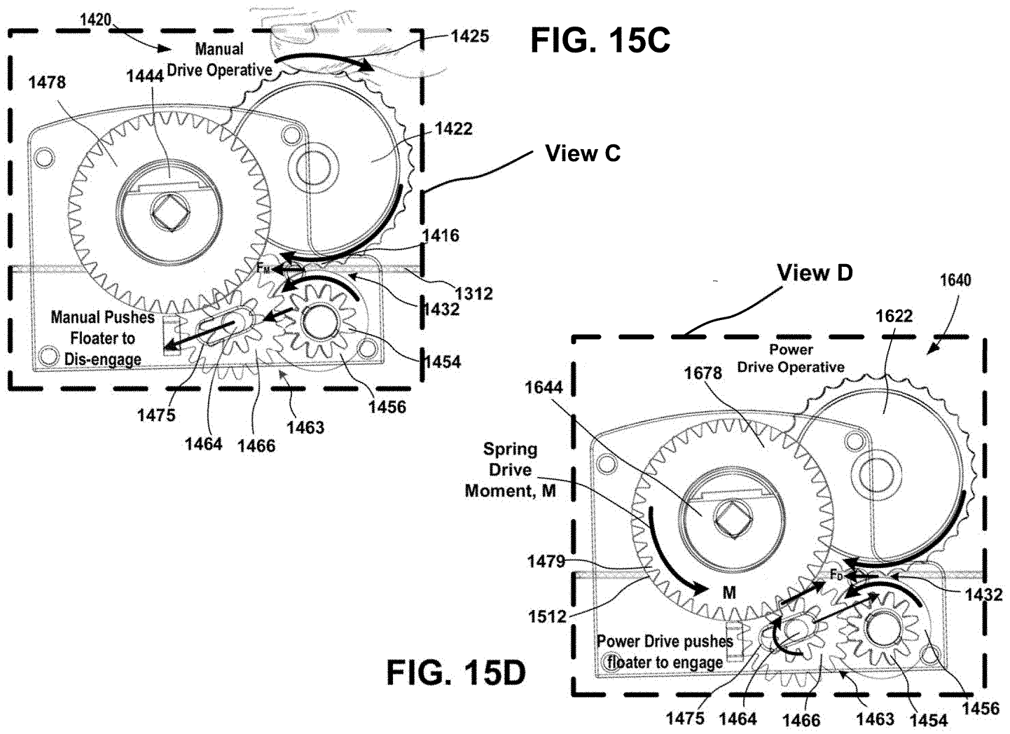

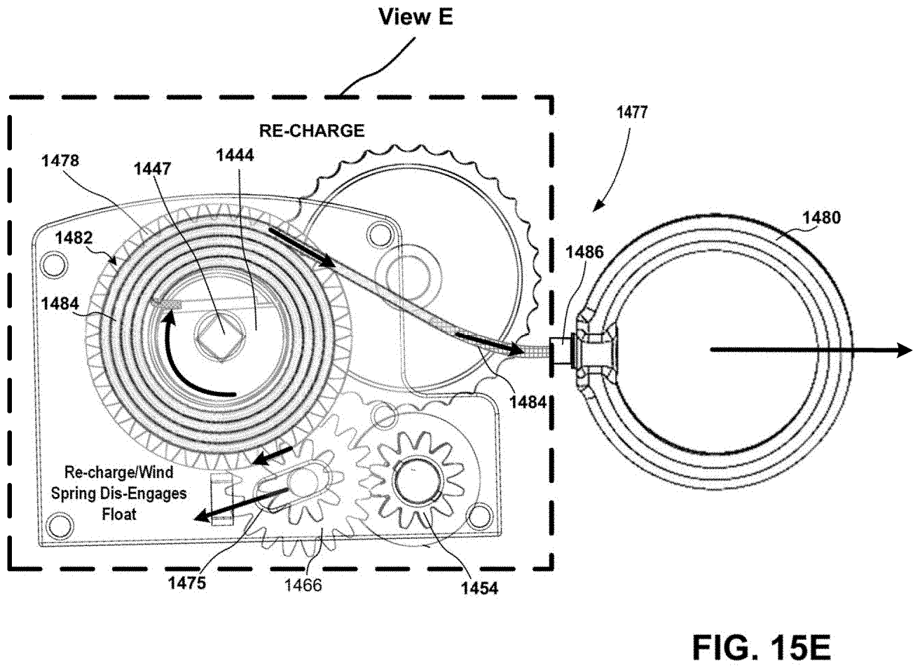

Referring now to FIGS. 14A, 14C, and 15B, manual drive 1420 generally includes a manual drive roller or thumbwheel 1422 and a manual control 1427 configured as a user-exposed user-controllable portion of the thumbwheel. The manual drive roller 1422 is rotatably coupled with the advancer body and has an engagement surface 1421 that is rotatable with the manual drive roller. The engagement surface 1421 includes a drive portion 1416 extending into the guide wire pathway 1320 and configured to drivingly engage the guide wire 1312. The drive portion 1416 is configured to transmit a manual control force (not shown) applied by the user to manual control 1427 (e.g., rolling force applied by the thumb of hand 1318 to the thumbwheel) as corresponding manual drive force 1217 imparted by manual drive roller drive portion 1416 to the guide wire 1312 via the integrated combination drive 1418 discussed below to advance the second distance 1368.

The automated drive 1440 generally includes a power driver 1442, a powered or automated drive roller 1456, and an actuator 1460. The power driver 1442 is configured to store potential energy for driving guide wire advancement of guide wire 1312 for the primary distance 1366 (FIG. 11) and transmit a corresponding automated drive force (not shown) to the guide wire via automated drive roller 1456 when actuated. The automated drive roller 1456 is rotatably coupled with the advancer body 1430 and has a resilient surface 1428 (FIG. 14C) rotatable with the automated drive roller. The resilient surface 1428 includes a drive portion 1429 extending into the guide wire pathway 1320 and configured to drivingly engage the guide wire 1312 to advance when driven by power driver 1442. The actuator 1460 is configured to be actuated by the single hand 1318 (FIG. 11 along with FIG. 15B) to activate the power driver 1442 and transmit the automated drive force (not shown) to the automated drive roller drive portion 1429.

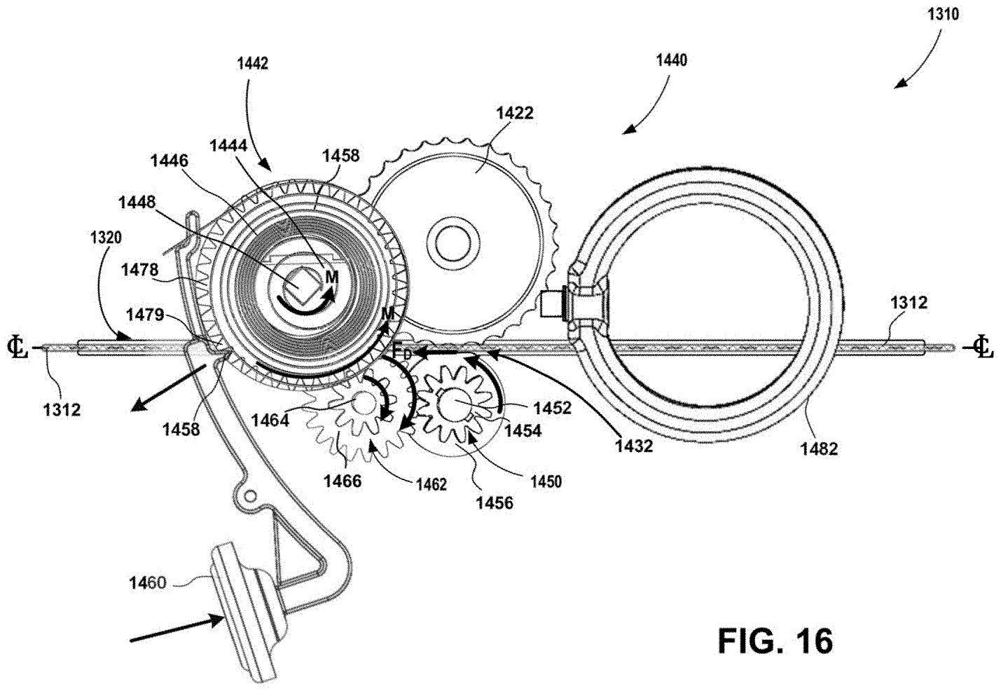

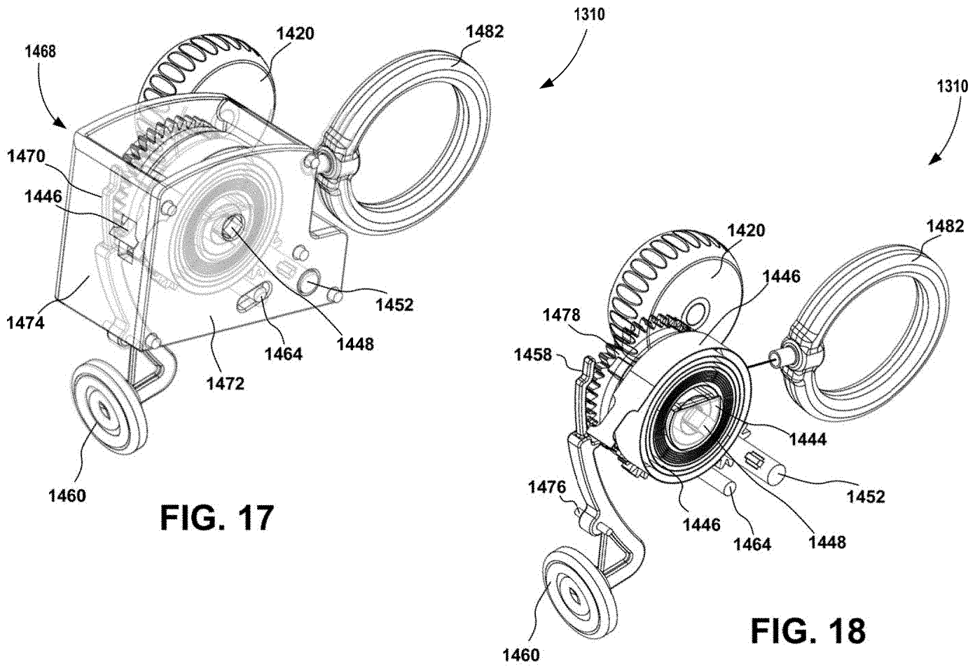

Referring now to FIGS. 15A, 15B and 16-19, the power driver 1442 generally includes a flat coil spring 1446, a spring hub 1444, a spring shaft 1448, a pulley gear 1478 and a gearbox 1468. As shown in FIG. 17, the gearbox 1468 includes a gearbox body 1470, a gearbox cover 1472, and a gearbox front 1474. The gearbox body 1470 forms a generally rectangular case that retains the flat coil spring 1446, the spring hub 1444, the spring shaft 1448, and the pulley gear 1478 in an operative arrangement for providing an automated drive force F (FIG. 16) for advancing the guide wire 1312 the primary distance 1366 when the actuator 1460 is actuator by the single hand 1318. The gearbox cover 1472 is a removable cover providing access to components retained therein, which extends across a left face of the gearbox and supports operative arrangements of the components in combination with the gearbox box body and front. For instance, gearbox cover 1472 defines an opening that supports a left end of the spring shaft 1448 and permits rotation of the same, and also supports a left end of drive shaft 1452 discussed further below along with the power driver 1442. Gearbox cover 1472 further supports a left end of a floater gear shaft 1464 within a floater guide slot 1475, which is discussed below along with the transmission switch 1463 and FIGS. 15C, D and E. The gearbox is attached to the guide wire advancer housing 1330 and oriented such that the gearbox front 1474 is disposed toward the front or distal end of the housing, and the gearbox cover is disposed on the left side of the advancer covered by left housing 1332. In this orientation within the guide wire housing 1330, the spring shaft 1448 is supported laterally across the gearbox 1468 in a generally left-to-right widthwise orientation of the guide wire advancer 1310 and is rotatably supported via an opening formed on the left side within gearbox cover 1472 and similarly supported on the right side by a corresponding opening within the gearbox body 1470.

Gearbox front 1474 defines a spring end retention opening therein, which retains the outer end of the coiled spring 1446 and thereby affixes one end of the flat coil spring to the guide wire body 1330. Referring to FIG. 16 along with FIG. 17, the flat coil spring 1446 is wound as a flat coil around spring hub 1444, which is disposed on the spring shaft 1448 that is rotatably supported across the inside of the gearbox 1468. The spring hub 1444 is interlocked with the spring shaft 1448, such that the spring shaft and spring hub rotate together about a longitudinal axis of the spring shaft. The second end of the flat coil spring 1446 disposed inside the coil is attached to the spring hub, whereas the opposite first end at the beginning of the coil and located outside the coil is attached to the gearbox front 1470, which is attached to the guide wire body 1330 as discussed above. Thus, any rotational forces generated via potential energy stored in flat coil spring 1446 are configured to induce rotation about the rotational axis of spring hub 1448 and spring shaft 1448 with respect to the gearbox 1468.

As shown in FIG. 18 along with FIGS. 16 and 17, pulley gear 1478 is also rotatably disposed on spring shaft 1448 adjacent to spring hub 1444 with the spring shaft extending through a central axis of the pulley gear and interlocked therewith, such that the pulley gear rotates about the axis of the spring shaft and along with rotation of the spring shaft. Thus, any rotational forces or moments applied by flat coil spring 1446 inducing rotation of spring hub 1444 about its central axis along with rotation of spring shaft 1448 also induce rotation of pulley gear 1478 therewith. When flat coil spring 1446 is wound tightly about spring hub 1444 such that it retains potential energy for driving the automated drive 1440 when actuated, spring 1446 applies a drive moment M in the rotational direction shown in FIG. 16 about spring shaft 1448 via spring hub 1444, which likewise applies moment M in the same direction upon pulley gear 1478 about the axis of the spring shaft. However, actuator 1460 is operatively coupled with a lock 1458 that engages pulley gear teeth 1479 located along a perimeter portion of the pulley gear 1478, which blocks or stops rotation of the pulley gear along with rotation of spring shaft 1448 connected thereto and prevents operation of the automated drive 1240 until lock 1458 is disengaged from the pulley gear teeth.

As shown in FIG. 17, actuator 1460 is pivotably connected with lock 1458 such that movement of actuator 1460 via a finger of the single hand 1318 moves lock 1458 in an opposite direction and simultaneously disengages the lock from engagement with pulley gear teeth 1479. Accordingly, absent engagement of lock 1458 with pulley gear 1478, pulley gear can freely rotate with pulley hub 1444 and pulley shaft 1448 about the axis of the pulley shaft according to the drive moment M transmitted to the pulley gear from flat coil spring 1446. Gear teeth 1479 are configured to engage corresponding gear teeth on floater gear 1466 when actuated to drive rotation of the floater gear along with forcing the floater gear into a rotational, driving engagement with drive gear 1454 as discussed below along with FIGS. 15C, D and E.

It is understood that the pitch radii of meshing gear teeth 1479, the sets of gear teeth disposed along floater gear 1466, and the gear teeth disposed along drive gear 1454 can vary with respect to each other according to various parameters and preferences, such as the rotational spring force provided by flat coil spring 1446 and duration of drive force when actuated, the desired primary advancement length 1366 for use of the guide wire advancer 1310 with a particular surgical device or types of devices, and/or pushability and related parameters for the particular guide wire and its usage. Drive chain and related modifications can fine tune operational characteristics of the automated drive 1440, such as mechanical advantage features, drive duration that can be provided by the stored potential energy and spring parameters, and drive forces applied to the guide wire 1312 for automated advancement.

For example, in the configuration shown in FIG. 16, pulley gear 1478 has a large pitch radius for its mesh teeth 1479 with respect to the comparatively small pitch radius of mesh teeth on floater gear 1466 engaged by mesh teeth 1479. As such, a single rotation of pulley gear 1478 drives multiple rotations of floater gear 1466 with a lesser force applied than if the pitch radius of mesh teeth 1479 were smaller. However, floater gear 1466 applies mechanical advantage benefits via transmitting moment applied to it by pulley gear 1478 to drive gear 1454 using an attached gear having a large pitch radius to mesh with the drive gear and increase the force applied to the gear teeth of drive gear 1454 by a corresponding mechanical advantage factor. In other words, the rate at which drive force can be applied via the flat coil spring for the amount of potential energy its stores and the force ultimately applied to the guide wire as drive force F can be modified and fine-tuned as appropriate for usage of the guide wire advancer 1310 with different types and configurations of surgical devices according to mechanical advantage, gear ratios and related drive train principles.

Referring to FIG. 16, the integrated advancement driver 1430 generally includes a cooperative feed roller nip 1432 configured to advance the guide wire in response to transmission of the automated drive force F_{D} or a manual drive force F_{M} applied by the user via thumbwheel 1422. In other words, the integrated advancement driver 1430 is configured as a multi-source advancement driver that can readily advance the guide wire according to the user's control movements including responsive to a user-exerted manual control or manual drive force on thumbwheel 1422, and in response to user-actuation of the automated driver 1440. As such, the cooperative feed roller nip 1432 includes a drive roller from both the manual drive 1420 and the automated drive 1440. Further, the cooperative feed roller nip 1432 is defined between the drive portion 1416 of the manual drive roller 1422 that extends into the guide wire pathway, which is configured in an opposed arrangement with the drive portion 1429 of the automated drive roller 1456 that likewise extends into the guide wire pathway. The opposing drive portions can be configured to engage opposite side regions of the guide wire and cooperate as a pair of feed rollers for advancing the guide wire 1312 along the pathway 1320 toward the second end portion 1325. Further, the opposing drive portions can each be configured to act as both a drive roller and a driven roller based on the drive roller that is acting as the operative drive providing drive force to the guide wire.

Referring now to FIGS. 15C and 15D, further details of the manual drive 1620 and the power drive 1640 as drive components of the integrated advancement drive 1430 are shown, as well as aspects and features pertaining to a transmission switch 1463 configured to automatically move between a first position D and a second position E. The transmission switch is configured to automatically move to the first position D when the user exerts manual control via thumbwheel 1422. The first position D de-couples the power drive 1440 from its driving connection with the automated or powered drive roller 1454 to prevent drive force inadvertently being applied by the power drive 1440 on the guide wire 1312 while the user applying fine-tuning user-controlled advancement of the guide wire. The transmission switch is further configured to automatically engage or connect the power driver 1440 to the powered drive roller 1454 when the user actuates the actuator to transmit powered drive force to the guide wire 1312. The transmission switch 1463 generally includes a floater gear 1466, a floater gear shaft 1464, and a pair of guide slots 1475 configured to support opposite ends of the floater gear shaft. The pair of corresponding guide slots 1475 are formed through the gearbox 1468 with one formed through the cover 1472 on the left side of the gearbox, and the other on the opposite right side through the gearbox body 1470. The opposing slots support opposite end portions of floater gear shaft 1464 such that the floater gear shaft extends laterally between left and right sides of the gearbox across the width of the guide wire advancer 1310. The floater gear shaft is supported within the opposing guide slots 1475 to allow rotation of floater gear shaft 1464 and sliding translation of the shaft along the slot.

Referring now to FIG. 15C, operation of transmission switch 1463 is shown while the manual drive 1420 of the integrated combination drive 1418 is operative, as well as operations of the manual drive. Guide wire advancer 1310 is configured such that the user applies control movements 1425 to the manual control 1427 of the thumbwheel 1422 using the thumb of single hand 1318 via thumb movements rearward or in a proximal direction, which rotates the exposed control portion rearward or in a proximal direction. An outer surface portion 1421 of thumbwheel 1422 can be configured to have with a knurled, abrasive, tactile, contoured or other type of surface texture or features providing high frictional engagement, such as with the user's thumb of single hand 1318 and/or for driving engagement of the guide wire 1312. A manual drive portion 1416 of outer surface portion 1421 extends into the guide wire channel 1320 and drivingly engages a side portion of the guide wire 1312 disposed within the nip 1432 to transmit a manual drive force F_{M} applied by the user to the guide wire. When the manual drive portion 1416 is applying manual force F_{M} to the guide wire, automated drive roller 1456 acts as a driven roller and correspondingly rotates in an opposite direction as thumbwheel 1422. Drive gear 1454 is attached to drive roller 1456 and, thus, correspondingly rotates along with the drive roller as shown in FIG. 15C. Mesh gear teeth disposed along drive gear 1454 engage corresponding mesh gear teeth of floater gear 1466 when the floater gear is proximate to or in engagement with the drive gear 1454 and push the floater gear 1466 along guide slots 1475 while rotating as shown responsive to application of a manual drive force. As such, floater gear 1466 automatically slides into the dis-engagement position D, which de-couples the pulley gear 1478 from engagement with drive gear 1454.

Referring now to FIG. 15D, operation of transmission switch 1463 is shown while the power drive 1440 of the integrated combination drive 1418 is operative, as well as further operations of the power drive. Guide wire advancer 1310 is configured such that the user can actuate actuator 1460 as discussed above to release potential energy stored by flat coil spring 1446 such that moment M applied to pulley gear 1478 rotates the pulley gear in the direction shown. As discussed above along with FIG. 16, gear teeth disposed along the pulley gear engage corresponding mesh teeth of floater gear 1466, which applies rotational force to rotate and translate the floater gear along guide slot 1475 into an engagement position E, in which the mesh gear teeth of the floater gear drivingly engage mesh gear teeth of drive gear 1454 to drive rotation of the drive gear in the drive direction shown. Rotation of drive gear 1454 as shown correspondingly rotates drive roller 1456 that is attached to the drive gear and exerts power drive force FT) upon guide wire 1312 disposed within the nip as discussed above along with FIG. 16. Thus, floater gear 1466 automatically slides into the engagement position E, which couples pulley gear 1478 into driving engagement with drive gear 1454 when the power drive is actuated. After potential energy stored in flat coil spring 1446 has been expended, the user can apply manual control via the thumbwheel and again disengage the floater as described above in FIG. 15C.

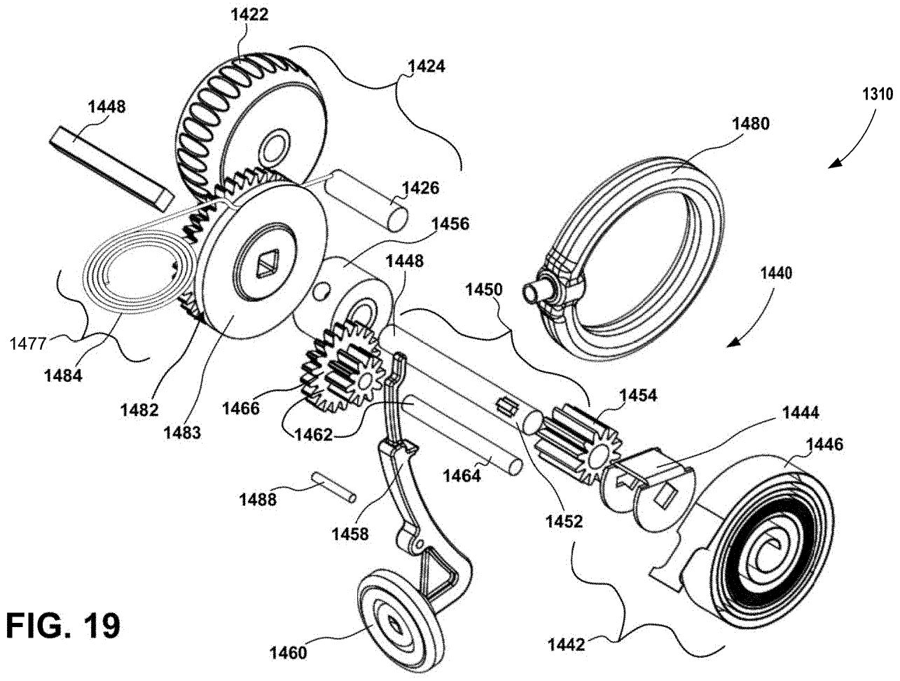

Referring now to FIG. 15E along with FIG. 19, guide wire advancer 1310 further includes a recharge assembly 1477 that allows the automated power drive 1440 to be reset or recharged along with rewinding the flat coil spring 1446, such that potential energy is restored for further actuations of the power drive. This option can allow guide wire advancer 1310 to be advantageously be used repeatedly for various circumstances, such as for usage with surgical devices having extended length channel pathways that exceed primary distance 1366, for re-advancing improperly executed guide wire advancement procedures or to switch guide wires, for using guide advancer to perform multiple procedures, or other reasons. Recharge assembly 1477 generally includes a pull tab 1480, a coil cavity 1482, a pull line 1484, and a pull line eyelet 1486. The coil cavity 1482 can be configured with pulley gear 1478 as a disk-shaped cavity adjacent to pulley gear 1478 bounded on one side by a side surface of the pulley gear and on the other side by a coil cavity wall (FIG. 19) configured as an additional disk shaped surface that is laterally offset from the pulley gear such that a generally disk shaped cavity is formed in which a pull line can be wrapped or coiled around spring shaft 1447 in a similar manner as flat coil spring 1446. A pull line 1484 is attached at a first end to spring hub 1444 similar to flat coil spring 1446, and is coiled around the spring hub in an opposite direction from the coil direction of the flat coil spring. An opposite second end of the pull line 1484 is thread through an eyelet 1486 as it exits a rearward or proximal portion of the guide wire housing 1330 and attached to a pull tab 1480 disposed outside of the guide wire housing at a distal end thereof.

As shown in FIG. 15E, pull tab 1480 is configured to be pulled in a proximal direction away from the guide wire advancer housing 1330, such as by the user via a second hand or by another person. As the pull tab is pulled proximally, the pull line 1484 uncoils and rotates the spring hub 1444 along with pulley gear 1478 and spring shaft 1447 in an opposite rotation direction from the direction of rotation imparted upon pulley gear 1478 when the actuator 1460 is actuated and the power drive 1440 becomes operative as discussed along with FIG. 16. Rotation of the spring hub 1444 in this opposite direction acts to rewind flat coil spring 1446 around the spring hub and restore its potential energy. Further, as indicated in FIG. 15E, while the pull tab is being extended and the spring rewound, pulley gear 1478 engages mesh teeth of the floater gear 1466 to disengage from driver gear 1454 and avoid inadvertently applying any drive forces. Once recharged, actuator 1460 can be moved outward in a reverse direction from actuation to rotate lock 1458 into engagement with pulley gear teeth 1479 and retain the rewound, stored potential energy arrangement of flat coil spring 1446 and power drive 1440 until actuation is desired.

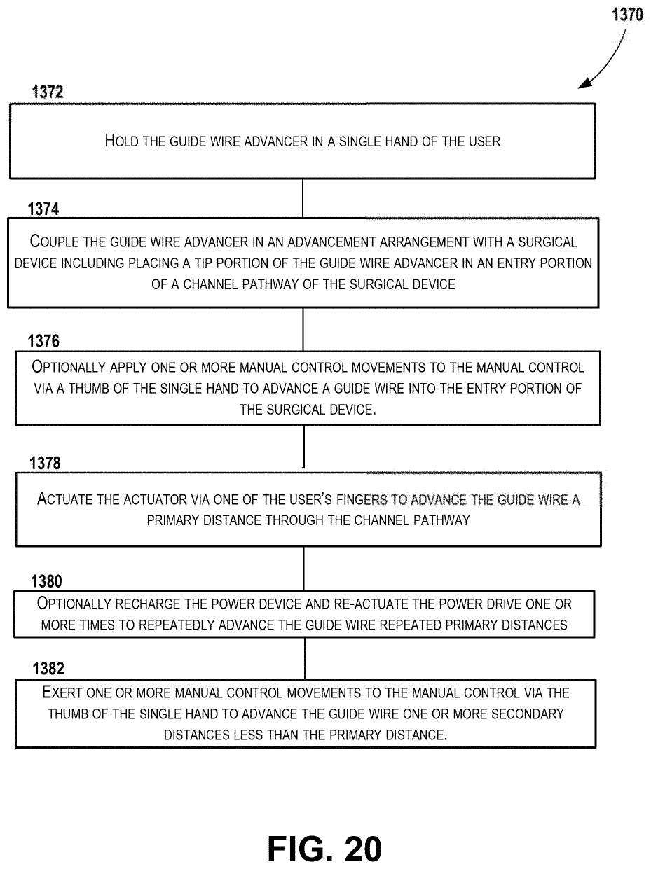

Referring now to FIG. 20 along with FIGS. 11-19, a method 1370 for advancing a guide wire using guide wire advancer 1310 with a guide wire 1312 and a surgical device, such as suture placement device 50 and/or other surgical device(s) is generally shown. The method 1370 generally includes step 1372 of holding the guide wire advancer 1310 in a single hand 1318 of a user and step 1374 of coupling the guide wire advancer 1310 in an advancement arrangement with a surgical device including placing a tip portion of the guide wire advancer in an entry portion of a channel pathway of the surgical device. The method 1370 optionally includes the step 1376 of applying one or more manual control movements to the manual control via a thumb of the single hand 1318 to advance a guide wire 1312 into the entry portion of the surgical device. The method 1370 further includes step 1378 of actuating the actuator 1460 via one or more of the user's fingers to advance the guide wire 1312 a primary distance through the channel pathway, and the optional step 1380 of recharging the automated power drive 1440 and re-actuating the power drive one or more additional times to repeatedly advance the guide wire 1312 repeated primary distances. The method 1370 also includes the step 1382 of exerting one or more manual control movements to the manual control via the thumb of the single hand to advance the guide wire one or more secondary distances less than the primary distance.

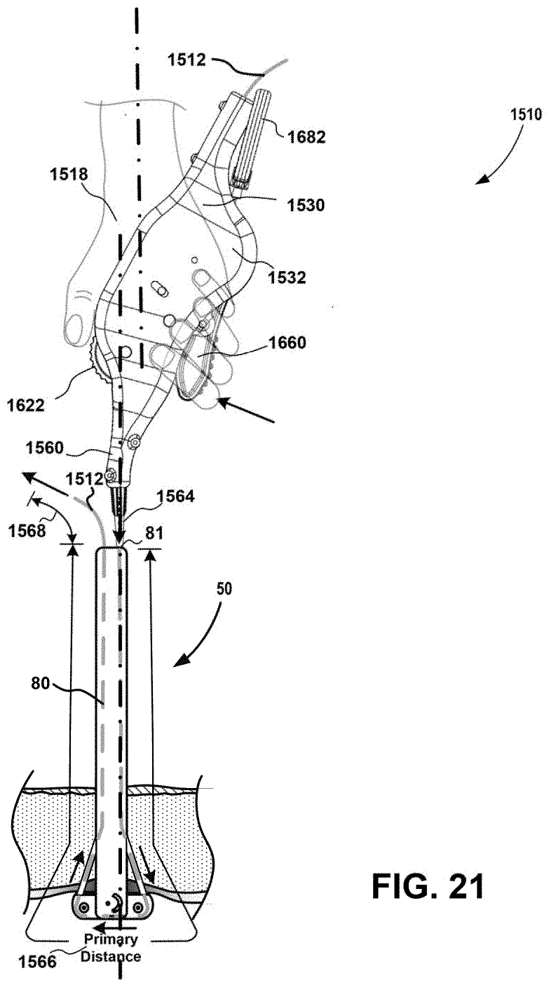

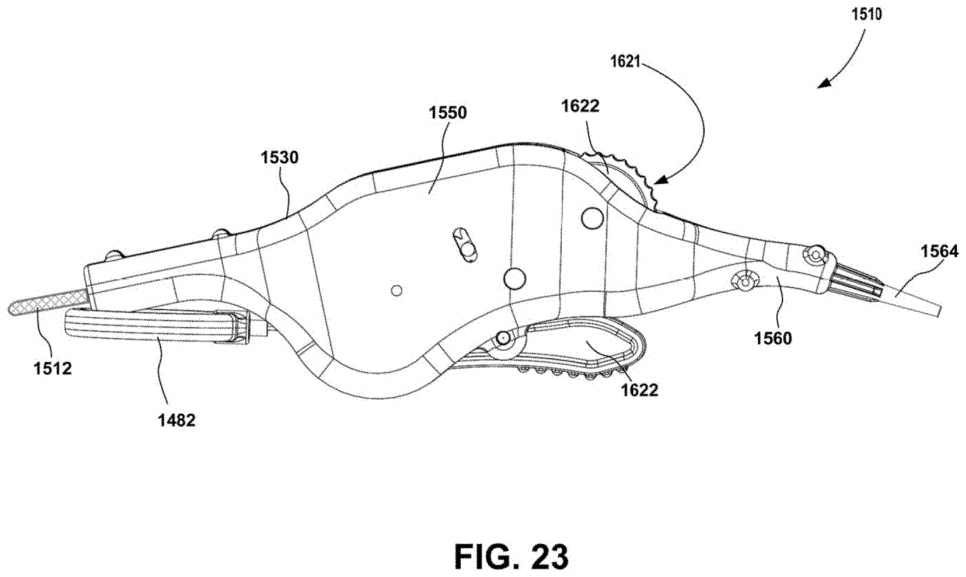

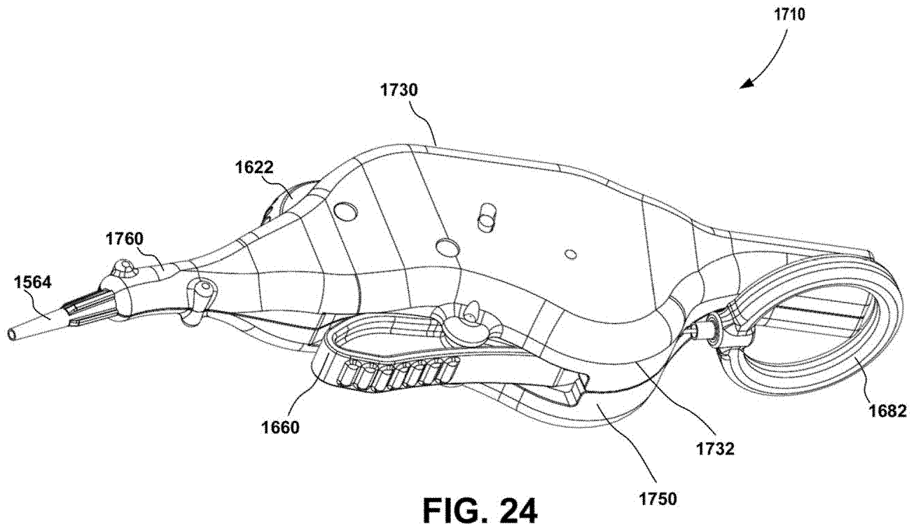

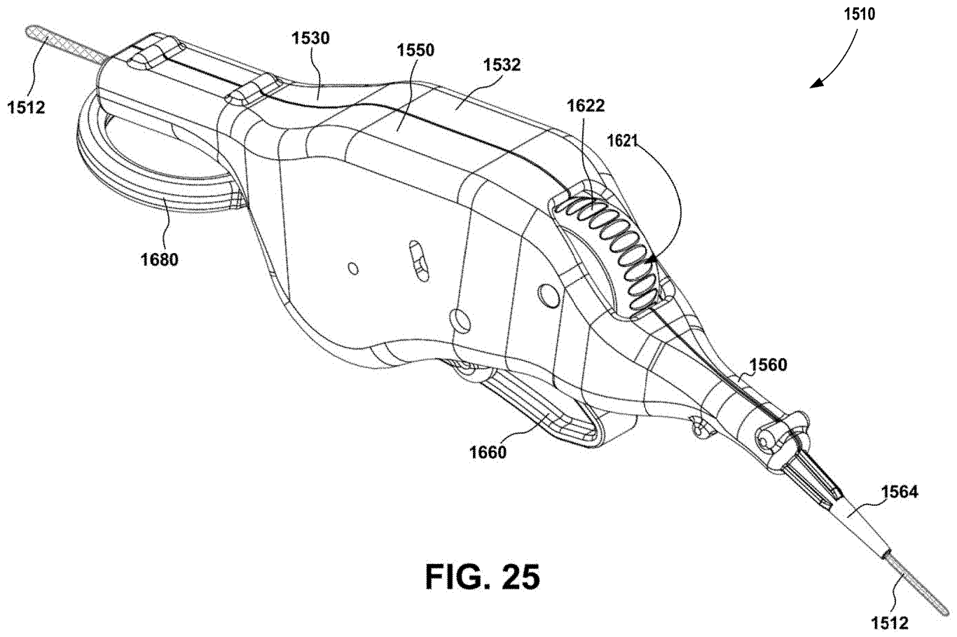

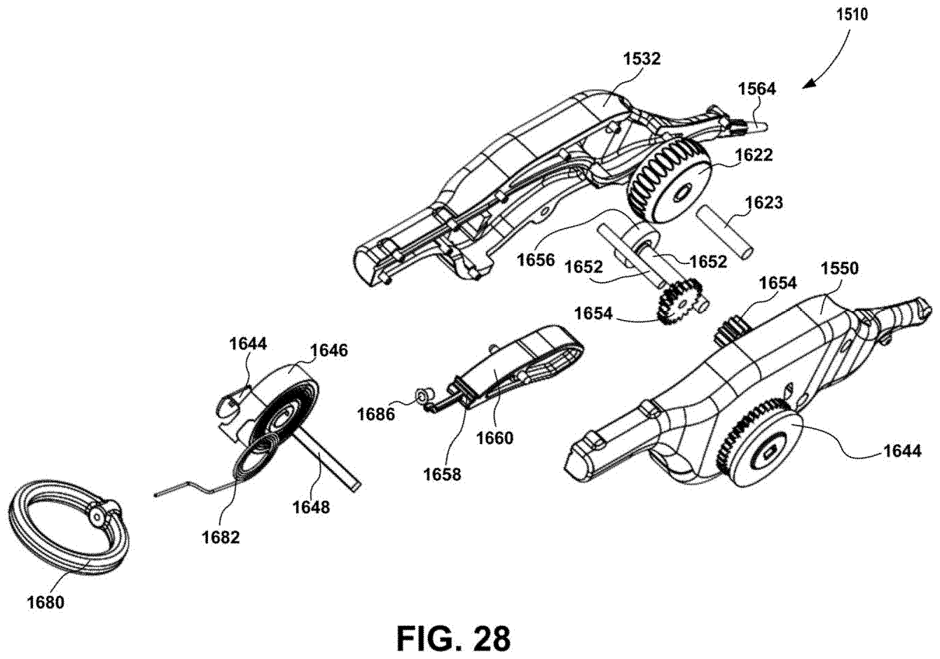

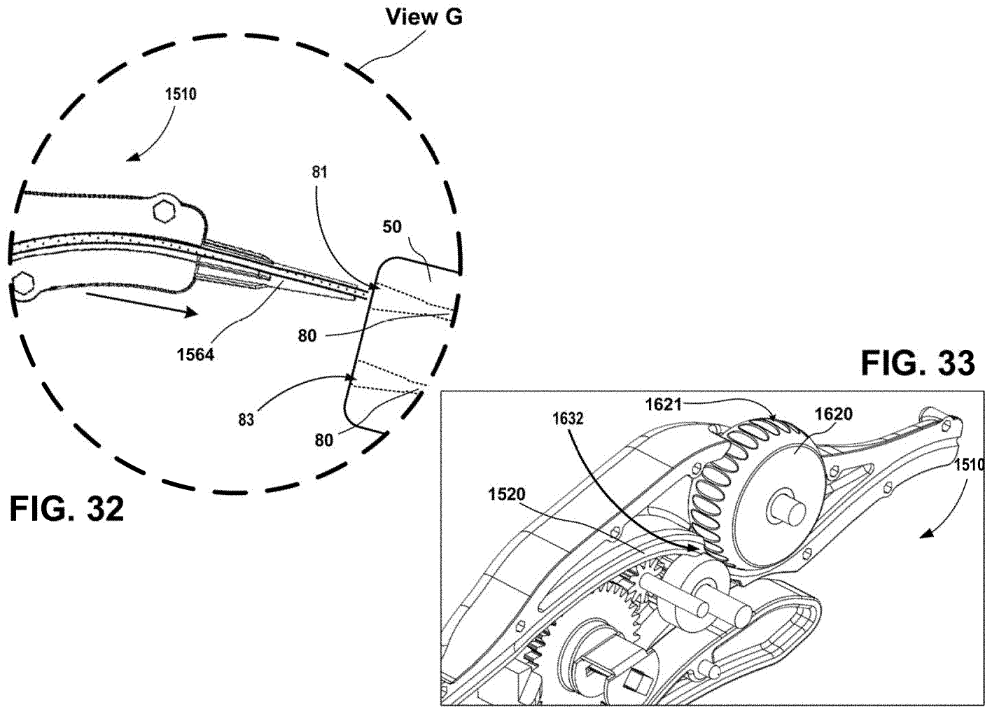

Referring now to FIGS. 21-37B and in particular to FIG. 21, a combination automatically-manually driven guide wire advancer 1510 is shown for advancing a guide wire an automated primary distance 1566 and a secondary manual distance 1568 that is less than the primary distance, which can be coupled with a guide wire/sheath coil (not shown) in a usage arrangement with the guide wire 1512 of the coil. It is understood that the combination guide wire advancer 1510 can be operatively configured with a surgical instrument including the suture placement device 50 discussed above along with FIGS. 3A-3E and shown in FIG. 21 while the surgical instrument is disposed within a port and prepared for suture placement procedures, as well as with other similar and non-similar types of surgical instruments (not shown). The combination guide wire advancer 1510 is configured to be held within the single hand 1518 of a user and easily controlled to perform guide wire advancement actions to support the surgical instrument, such as suture placement device 50 discussed.

The combination guide wire advancer 1510 includes many aspects and features that are the same as or similar to those discussed above with guide wire advancers 910, 1110, and 1310. Accordingly, like numbers refer to like features. However, guide wire advancer 1510 further includes aspects and features not discussed along with guide wire advancers 910, 1110, and 1310 or that differ therefrom, which are discussed below and/or shown in FIGS. 21-40. Combination guide wire advancer 1510 is advantageously configured for the user to actuate an automatic drive and readily advance the guide wire a primary first distance 1566 based on potential energy stored therein. Such a configuration can provide a powered drive force to automatically advance the guide wire through a pathway of a coupled surgical device being supported by the guide wire advancer, like advancing the guide wire within a channel pathway 80 formed within the suture placement device 50. The combination guide wire advancer 1510 is further configured as a compact, ergonomic, high-power advancement device arranged for the user to readily actuate the automatic drive using a single hand 1518 gripping the guide wire advancer. For example, as shown in FIG. 21, movement of one or more fingers of the gripping hand 1518 can readily actuate the automatic drive by depressing an actuator 1660. Further, the combination guide wire advancer 1510 is advantageously configured for the user to exert control movements via the same gripping hand 1518 while gripping the guide wire advancer, such as via rolling thumb movements exerted on a manual control 1627 conveniently placed proximate the user's thumb. As such, the user can readily grip the advancer and easily control advancement operations including both an automated primary advancement of the guide wire and manual, fine-tune secondary advancement of the guide wire.

Further, combination guide wire advancer 1510 is configured as an ambidextrous device that can perform and be used equally well via a user's left or right. In addition, guide wire advancer 1510 is configured for ergonomic handling and extended usage such that it can be easily held and controlled in a single left or right hand of user, and maintains a generally neutral wrist angle during use along with having a comfortable form-fitting pistol type grip that can be held comfortably during extended usage. Combination guide wire advancer 1510 includes an angled connection for coupling with a guide wire supply and a flexible stylet tip connector for coupling with a surgical device, which increases the range of flexibility for comfortably using the guide wire advancer and coupling with related devices. In addition, guide wire advancer 1510 is configured to include a highly compact, yet high-power mechanical power source disposed within a small, lightweight envelope that provides enhanced flexibility and comfort for holding and using the advancer device with various devices, environments and uses. Moreover, guide wire advancer 1510 is configured for quick and easy recharging to enable multiple, repeated power drive actuations.

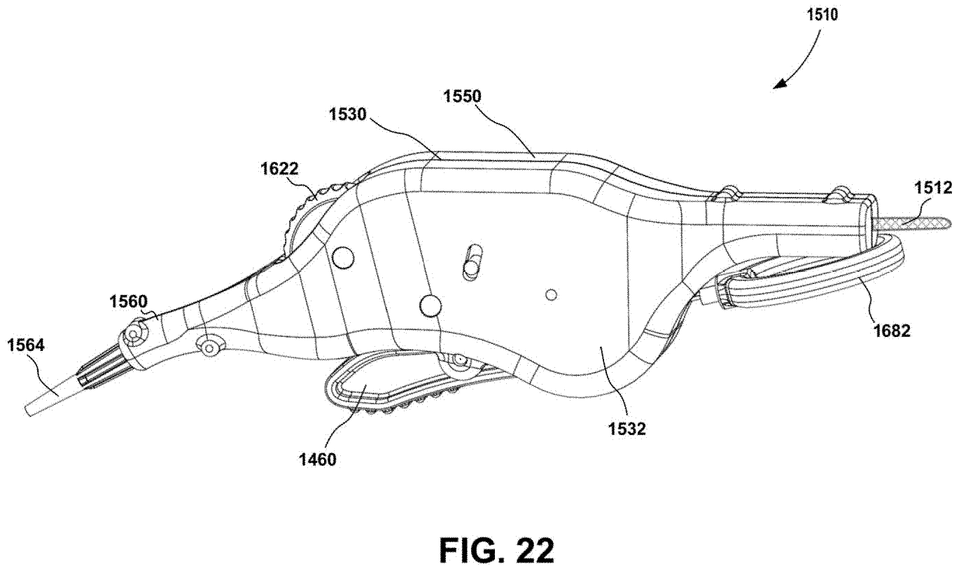

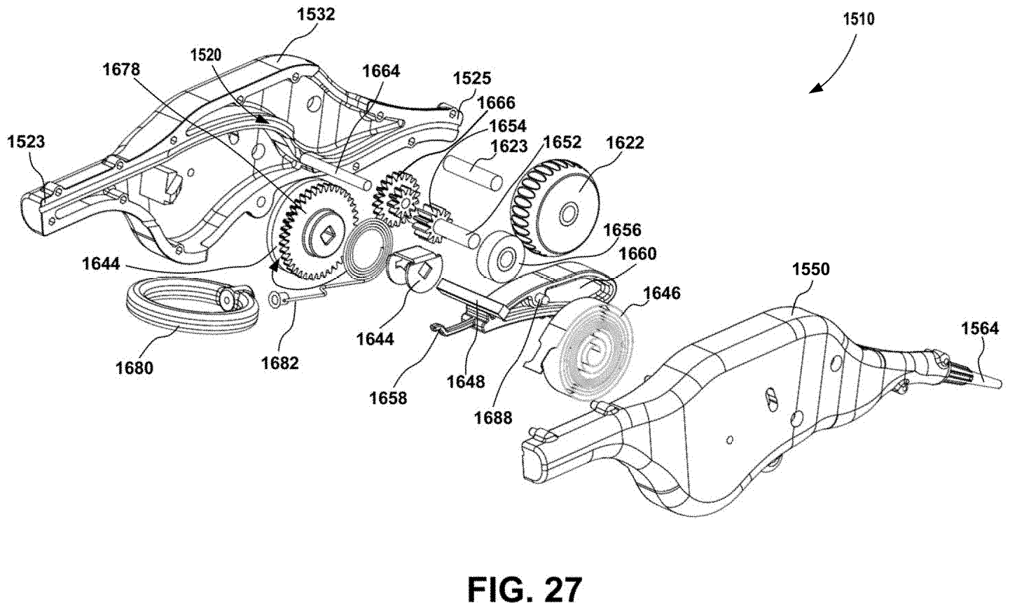

Referring now to FIGS. 22-27, the combination automatically-manually driven guide wire advancer 1510 generally includes an advancer body 1530, an automated power drive 1640, a manual drive 1620, and an integrated advancement driver 1630. The advancer body 1530 defines a guide wire pathway 1520 (FIG. 27) for advancing the guide wire 1512 therethrough from a first end portion 1523 to a second end portion 1525 of the advancer body, in which the advancer body is configured to be held in a grip of a single hand 1518 of a user and controllable by the single hand, for which guide wire advancer 1510 is configured to be held and used equally well by a left or right hand. The first end portion 1523 is configured to couple with the guide wire 1512 to receive a tip portion of the guide wire therein through an entrance of the pathway 1520 disposed at the first end portion 1523, and the guide wire 1512 is configured to advance through the pathway and out an exit of the pathway disposed at the second end portion 1525. The exit 1524 can include a stylet tip 1564 or similar arrangement for coupling the guide wire advancer in a support arrangement with a surgical device, such as suture placement device 50.

The advancer body 1530 includes a left housing 1532, a right housing 1550, and an introducer or stylet 1560 integrally formed within portions of both the left and right housings. As such, the left and right housing are generally mirror images of each other, controls are generally centered, and couplings or connections with other devices such as a guide wire 1512 or surgical device are arranged for ambidextrous usage such that guide wire advancer 1512 can be used equally well via a left or right hand. The guide wire pathway 1520 is formed through the stylet 1560 portion of the body disposed near the second end portion or distal portion of the device. However, the guide wire pathway 1520 follows a curved pathway as shown in FIG. 27, which provides significant benefits to user related ease of handling and controlling, compact size, and comfortable, ergonomic usage, as well as benefits for enhanced control and advancement of the guide wire.

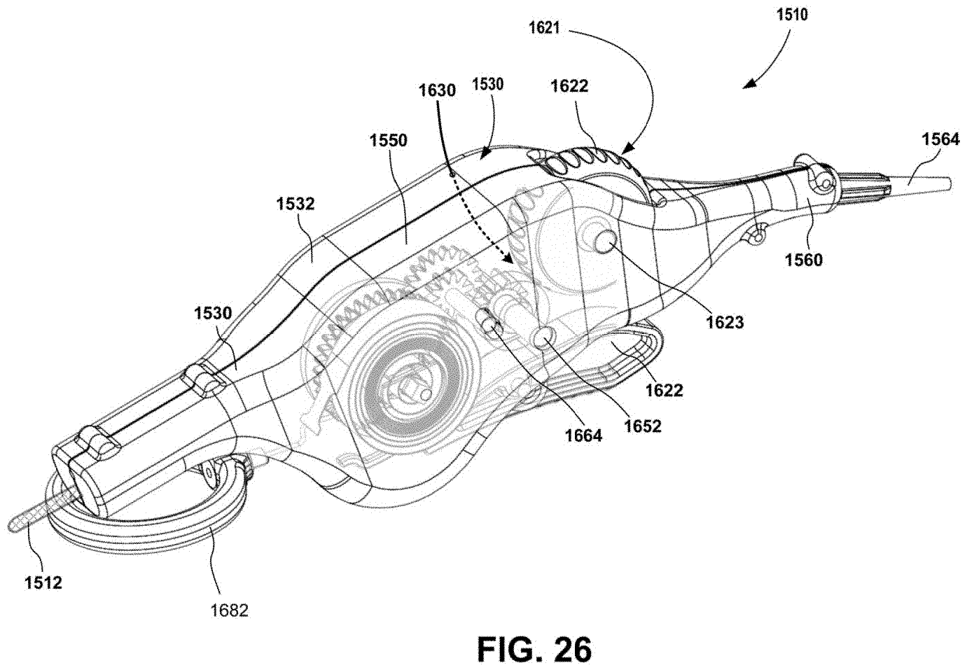

The integrated advancement driver 1630 (FIGS. 26 &27) is disposed within a front portion of the body 1530 proximate thumbwheel 1622 and along the guide wire pathway 1520 prior to traversing through the stylet 1560. As such, the guide wire advancer 1510 is configured to apply drive forces to the guide wire 1512 as it enters the stylet portion and enters the corresponding surgical device. The guide wire advancer 1510 is configured to closely support and guide the guide wire within the guide wire pathway 1520 along a curved pathway that prevents the guide wire from bending, kinking or folding over on itself along with enabling a compact, comfortable, high-power drive advancer arrangement.

As best seen in FIG. 21, upon actuation of actuator 1660, the automated power drive 1640 is configured to drive guide wire advancement for the primary distance 1566, which can be configured to correspond with substantially the entire length of the channel pathway 80 through the corresponding surgical device 50. The manual drive 1620 is configured to drive guide wire advancement for the secondary distance 1568 based on a user-exerted manual control movement on control 1627 that is configured as an exposed portion of a thumbwheel 1622. The exposed control 1627 of thumbwheel 1622 is configured to receive a user-exerted manual control movement (not shown) from the single hand 1518, such as rolling thumbwheel 1622, to transmit a corresponding manual drive force to the guide wire 1512 via the manual drive 1620 as discussed further below along with advancement of the guide wire 1512 the second distance 1568. One or more user-exerted manual control movements (not shown) can be applied by rolling the exposed control portion 1627 of thumbwheel 1622 repeatedly, which can beneficially be applied after actuation of the power drive to advance the guide wire the primary distance 1566, such as for manually advancing the guide wire tip portion 1516 to extend a desired length out of the surgical device. Manual control movements (not shown) can also be applied prior to actuation of the power drive, such as for manually advancing the guide wire tip portion 1516 through the length of the guide wire pathway and extend from the exit 1524 a desired length, such as for ensuring proper alignment and mating with a surgical device including extending into guide wire entry port 81 of suture placement device 50.

Thus, the combination of manual drive 1620 and automated power drive 1640 provides the user with significant control for advancing the guide wire as desired when aligning and mating the guide wire advancer 1510 with a surgical device, for quick and efficient deployment of the guide wire to advance through the pathway of the surgical device, and for arranging the guide wire for surgical procedures after deployment through the surgical device pathway. In addition, guide wire advancer 1510 is configured to efficiently advance the guide wire through and out of the advancer with minimal opportunities for the guide wire to bend, kink or fold over. These advantages are provided in compact device configured to be held and controlled via the single hand 1518 of a user that works well with either left or right hands, which can further be re-set or re-charged as discussed further below to provide multiple actuations of the power drive as appropriate for use, for instance, with surgical devices having extended channel pathways, and for multiple uses with the same or similar surgical devices including multiple suture placement devices for performing multiple port closure procedures. Many of these advantages and other benefits provided by guide wire advancer 1510 are related to the advantageous integration and combination of drives within the guide wire advancer including manual drive 1620, automated power drive 1640, and integrated combination drive 1630.

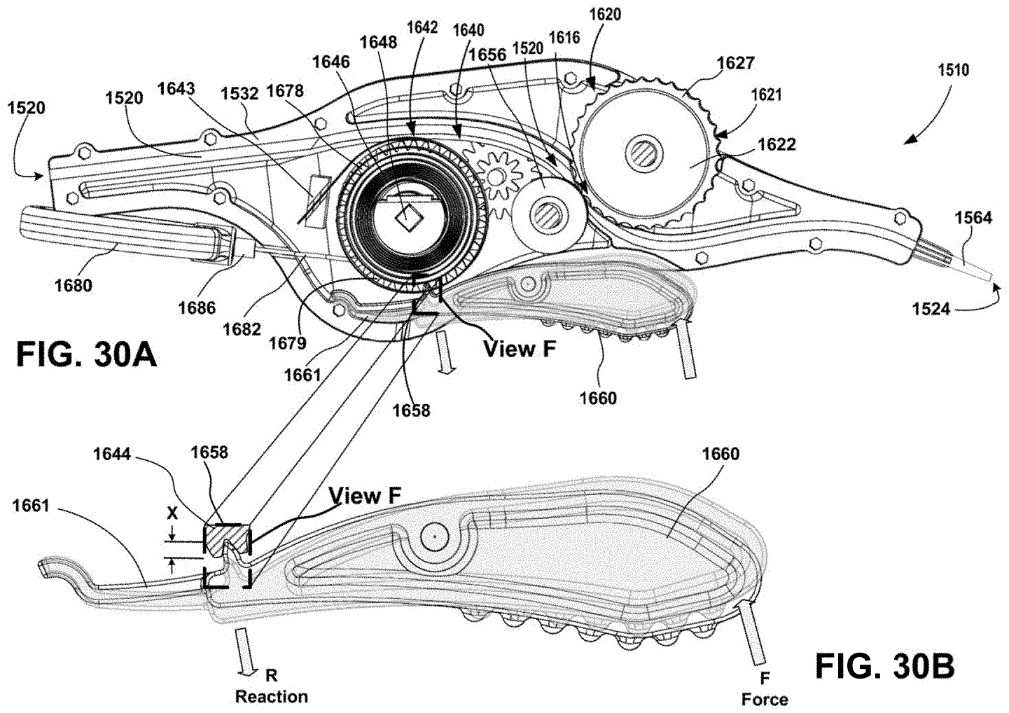

Referring now to FIG. 30A, manual drive 1620 generally includes a manual drive roller or thumbwheel 1622 and a manual control 1627 configured as a user-exposed user-controllable portion of the thumbwheel. The manual drive roller 1622 is rotatably coupled with the guide wire advancer body and has an engagement surface 1621 that is rotatable with the manual drive roller. The engagement surface 1621 includes a drive portion 1616 extending into the guide wire pathway 1520 and configured to drivingly engage the guide wire 1512 (not shown in FIG. 30A). The drive portion 1616 is configured to transmit a manual control force (not shown) applied by the user to manual control 1627 (e.g., rolling force applied by the thumb of hand 1518 to the thumbwheel) as a corresponding manual drive force imparted by manual drive roller drive portion 1616 to the guide wire 1512 via the integrated combination drive 1618 as discussed below along with the integrated combination drive to advance the guide wire the second distance 1568.

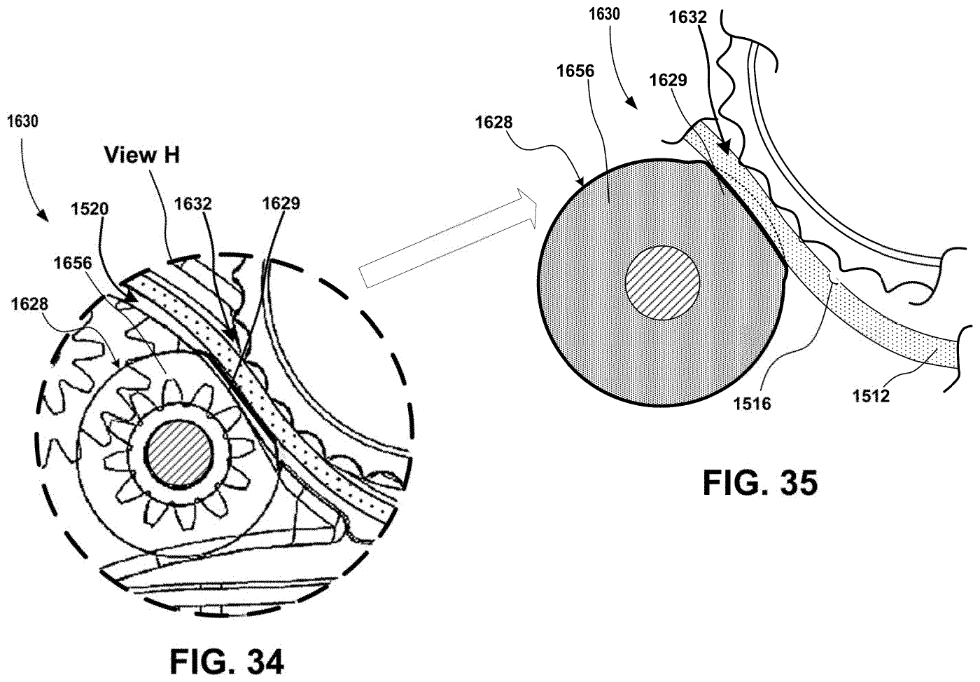

The automated drive 1640 generally includes a power driver 1642, a powered or automated drive roller 1656, and an actuator 1660. The power driver 1642 is configured to store potential energy for driving guide wire advancement of guide wire 1512 for the primary distance 1566 (FIG. 21) and transmit a corresponding automated drive force to the guide wire via automated drive roller 1656 when actuated. The automated drive roller 1656 is rotatably coupled with the advancer body 1630 and has a resilient surface 1628 (FIGS. 34 & 35) rotatable with the automated drive roller. The resilient surface 1628 includes a drive portion 1629 extending into the guide wire pathway 1520 and configured to drivingly engage the guide wire 1512 to advance when driven by power driver 1642. The actuator 1660 is configured to be actuated by the single hand 1518 (FIG. 21) to activate the power driver 1642 and transmit the automated drive force (not shown) to the automated drive roller drive portion 1629.

Referring now to FIGS. 26-30B, the power driver 1642 generally includes a flat coil spring 1646, a spring hub 1644, a spring shaft 1648, and a pulley gear 1678 and a gearbox 1668. Inner portions of the left cover 1532 and the right cover 1550 include support features defined therein or therethrough for retaining drive components in their operative arrangement when the left and right cover 1532 and 1550 are attached to each with drive components arranged therein, which is generally shown in FIG. 26. The flat coil spring 1646, the spring hub 1644, the spring shaft 1648, and the pulley gear 1678 when configured in their operative arrangement such as shown in FIGS. 26, 29 and 30B can provide an automated drive force for advancing the guide wire 1512 the primary distance 1566 when the actuator 1660 is actuator by the single hand 1518.

Left cover 1532 and right cover 1550 define a spring end retainer (FIG. 30A), which retains the outer end of the coiled spring 1646 and thereby affixes one end of the flat coil spring to the guide wire body 1530. The flat coil spring 1646 is wound as a flat coil around spring hub 1644, which is disposed on the spring shaft 1648 that is rotatably supported across the inside width of the guide wire advancer by support features formed on inner portions of left cover 1532 and right cover 1550. The spring hub 1644 is interlocked with the spring shaft 1648, such that the spring shaft and spring hub rotate together about a longitudinal axis of the spring shaft. The second end of the flat coil spring 1646 disposed inside the coil is attached to the spring hub, whereas the opposite first end at the beginning of the coil and located outside the coil is attached to the housing 1530 via spring end retainer 1643 (FIG. 30A) as discussed above. Thus, any rotational forces generated via potential energy stored in flat coil spring 1646 are configured to induce rotation about the rotational axis of spring hub 1648 and spring shaft 1648.

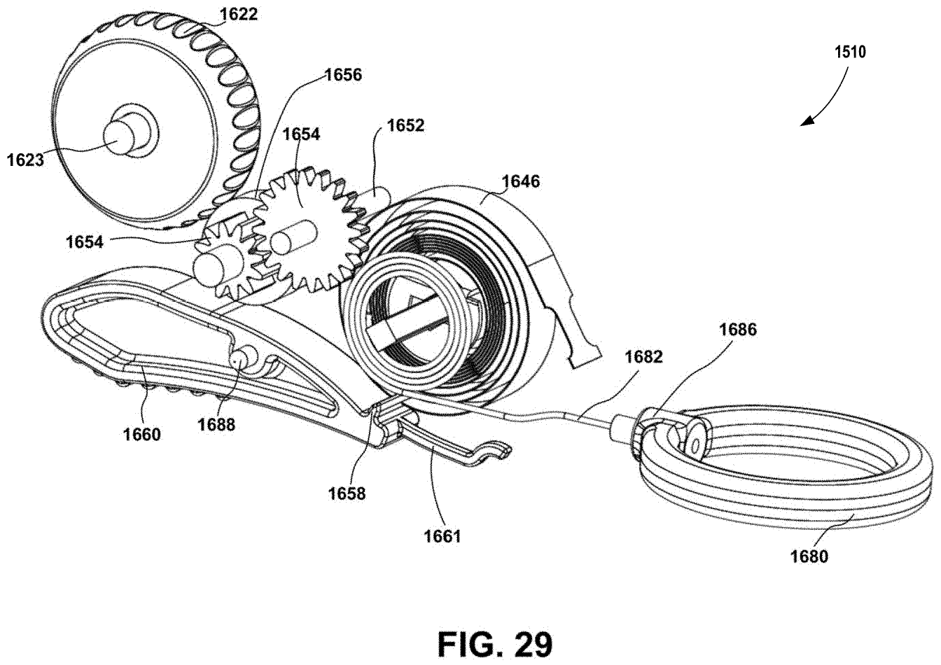

As shown in FIGS. 29 and 30A, pulley gear 1678 is also rotatably disposed on spring shaft 1648 adjacent to spring hub 1644 with the spring shaft extending through a central axis of the pulley gear and interlocked therewith, such that the pulley gear rotates about the axis of the spring shaft and along with rotation of the spring shaft. Thus, any rotational forces or moments applied by flat coil spring 1646 inducing rotation of spring hub 1644 about its central axis along with rotation of spring shaft 1648 also induce rotation of pulley gear 1678 therewith. When flat coil spring 1646 is wound tightly about spring hub 1644 such that it retains potential energy for driving the automated drive 1640 when actuated, spring 1646 applies a drive moment about spring shaft 1648 via spring hub 1644, which likewise applies the drive moment M to pulley gear 1678 about the axis of the spring shaft. However, as shown in FIGS. 30A and 30B, actuator 1660 is operatively coupled with a lock 1658 that engages pulley gear teeth 1679 located along a perimeter portion of the pulley gear 1678, which blocks or stops rotation of the pulley gear along with rotation of spring shaft 1648 connected thereto and prevents operation of the automated drive 1240 until lock 1658 is disengaged from the pulley gear teeth.

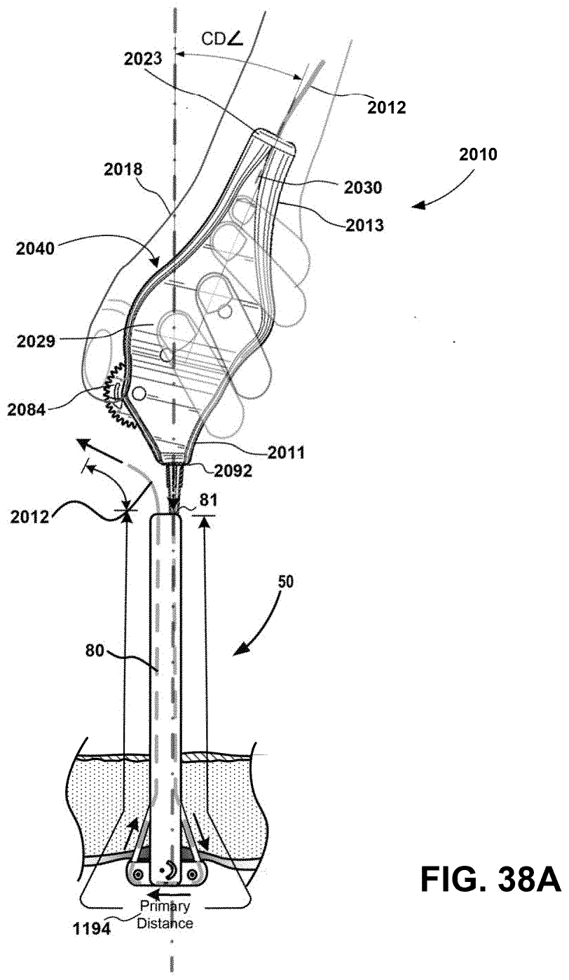

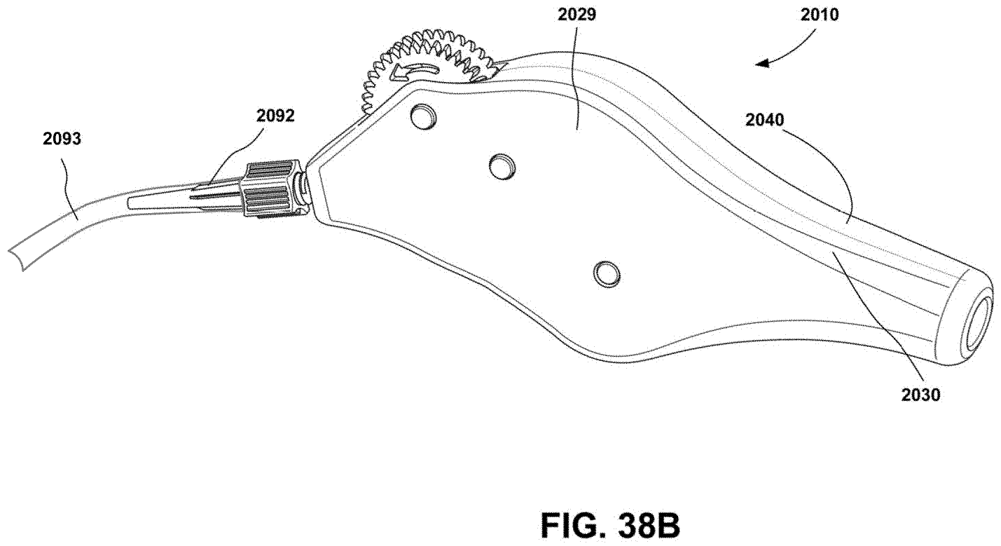

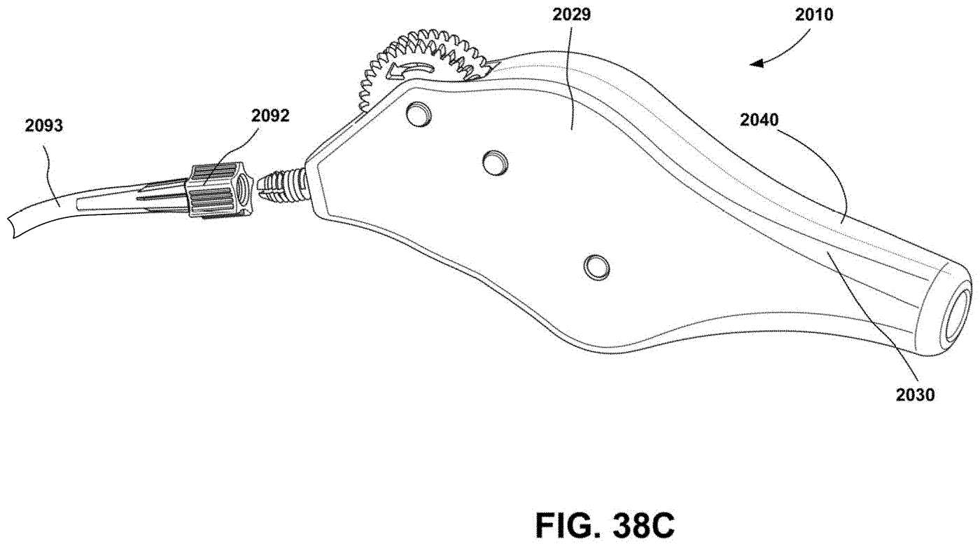

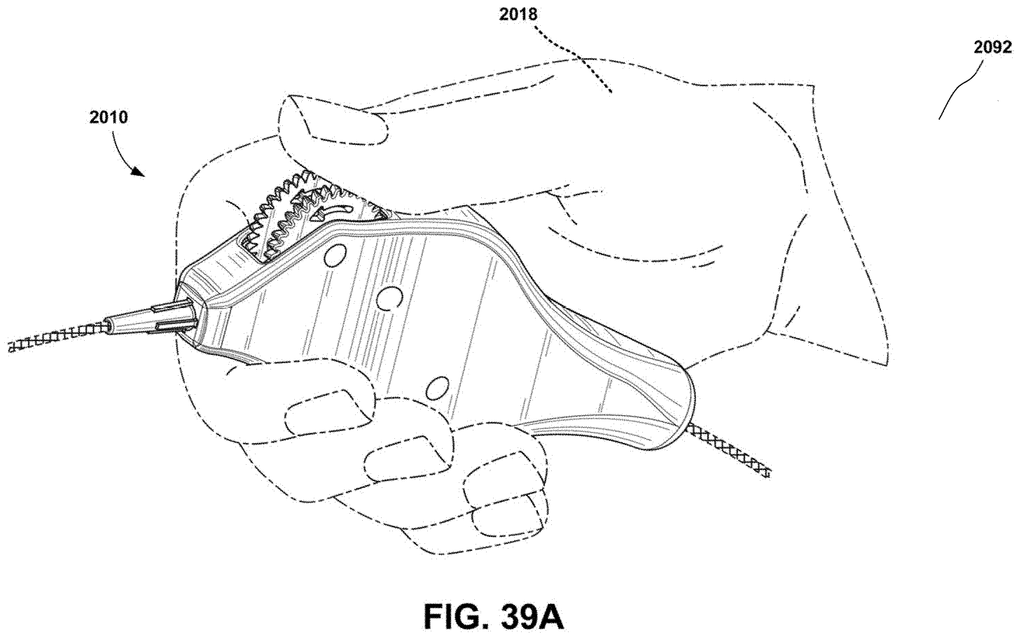

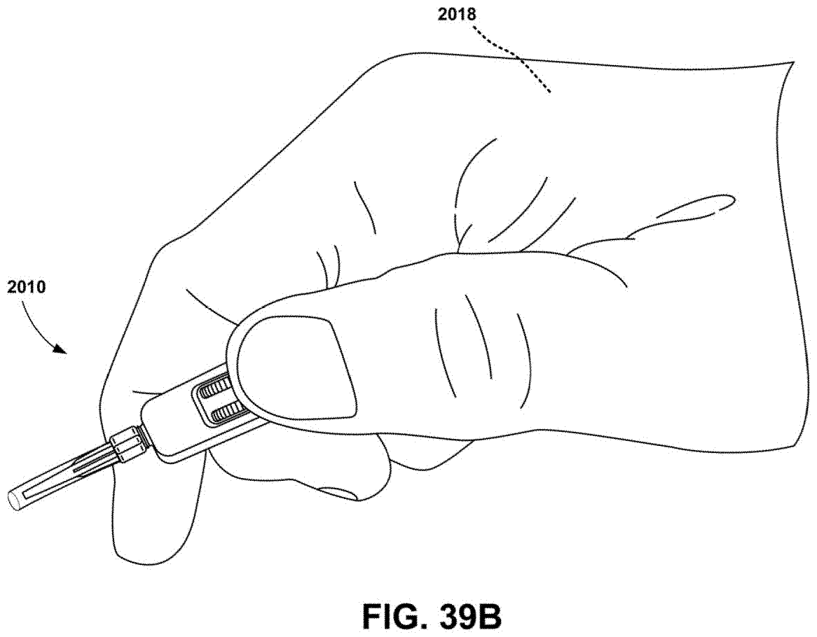

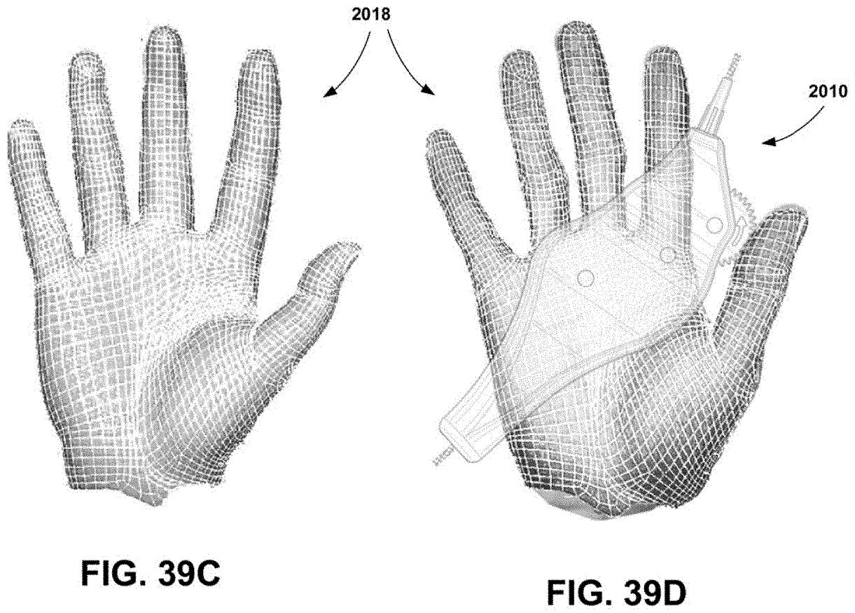

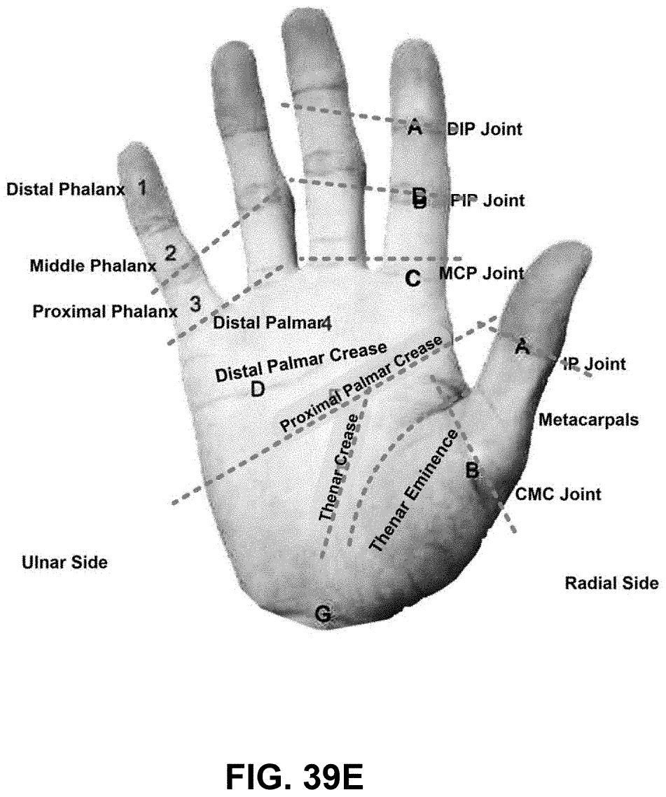

As shown in FIG. 30B, actuator 1660 is pivotably connected with lock 1658 such that movement of actuator 1660 via a finger of the single hand 1518 moves lock 1658 in an opposite direction and simultaneously disengages the lock from engagement with pulley gear teeth 1679. Accordingly, absent engagement of lock 1658 with pulley gear 1678, pulley gear can freely rotate with pulley hub 1644 and pulley shaft 1648 about the axis of the pulley shaft according to the drive moment M transmitted to the pulley gear from flat coil spring 1646. Gear teeth 1679 are configured to engage corresponding gear teeth on floater gear 1666 when actuated to drive rotation of the floater gear along with forcing the floater gear into a rotational, driving engagement with drive gear 1654 as discussed below along with FIGS. 38 and 39.

It is understood that the pitch radii of meshing gear teeth 1679, the sets of gear teeth disposed along floater gear 1666, and the gear teeth disposed along drive gear 1654 can vary with respect to each other according to various parameters and preferences, such as the rotational spring force provided by flat coil spring 1646 and duration of drive force when actuated, the desired primary advancement length 1566 for use of the guide wire advancer 1510 with a particular surgical device or types of devices, and/or pushability and related parameters for the particular guide wire and its usage. Drive chain and related modifications can fine tune operational characteristics of the automated drive 1640, such as mechanical advantage features, drive duration that can be provided by the stored potential energy and spring parameters, and drive forces applied to the guide wire 1512 for automated advancement.

For example, in the configuration shown in FIGS. 38 and 39, pulley gear 1678 has a large pitch radius for its mesh teeth 1679 with respect to the comparatively small pitch radius of mesh teeth on floater gear 1666 engaged by mesh teeth 1679. As such, a single rotation of pulley gear 1678 drives multiple rotations of floater gear 1666 with a lesser force applied than if the pitch radius of mesh teeth 1679 were smaller. However, floater gear 1666 applies mechanical advantage benefits via transmitting moment applied to it by pulley gear 1678 to drive gear 1654 using an attached gear having a large pitch radius to mesh with the drive gear and increase the force applied to the gear teeth of drive gear 1654 by a corresponding mechanical advantage factor. In other words, the rate at which drive force can be applied via the flat coil spring for the amount of potential energy its stores and the force ultimately applied to the guide wire as a drive force can be modified and fine-tuned as appropriate for usage of the guide wire advancer 1510 with different types and configurations of surgical devices according to mechanical advantage, gear ratios and related drive train principles.

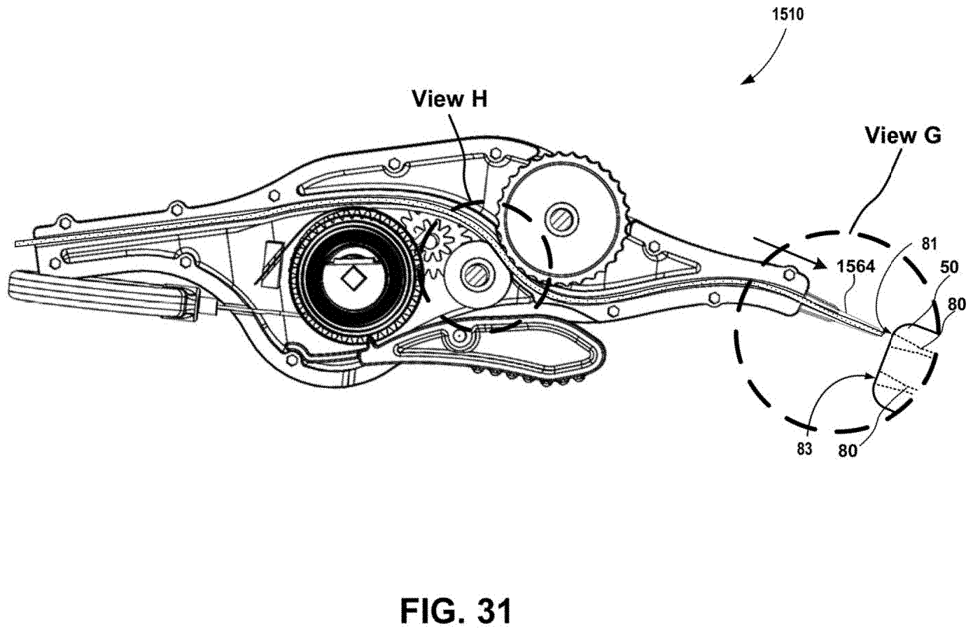

Referring to FIGS. 31-35 and, in particular, to FIGS. 34 and 35, the integrated advancement driver 1630 generally includes a cooperative feed roller nip 1632 configured to advance the guide wire in response to transmission of an automated drive force from automated power drive 1640 or a manual drive force applied by the user via thumbwheel 1622 and manual drive 1620. In other words, the integrated advancement driver 1630 is configured as a multi-source advancement driver that can readily advance the guide wire according to the user's control movements including responsive to a user-exerted manual control or manual drive force on thumbwheel 1622, and in response to user-actuation of the automated driver 1640. As such, the cooperative feed roller nip 1632 includes a drive roller from both the manual drive 1620 and the automated drive 1640. Further, the cooperative feed roller nip 1632 is defined between the drive portion 1616 of the manual drive roller 1622 that extends into the guide wire pathway, which is configured in an opposed arrangement with the drive portion 1629 of the automated drive roller 1656 that likewise extends into the guide wire pathway. The opposing drive portions can be configured to engage opposite side regions of the guide wire and cooperate as a pair of feed rollers for advancing the guide wire 1512 along the pathway 1520 toward the second end portion 1525. Further, the opposing drive portions can each be configured to act as both a drive roller and a driven roller based on the drive roller that is acting as the operative drive providing drive force to the guide wire.

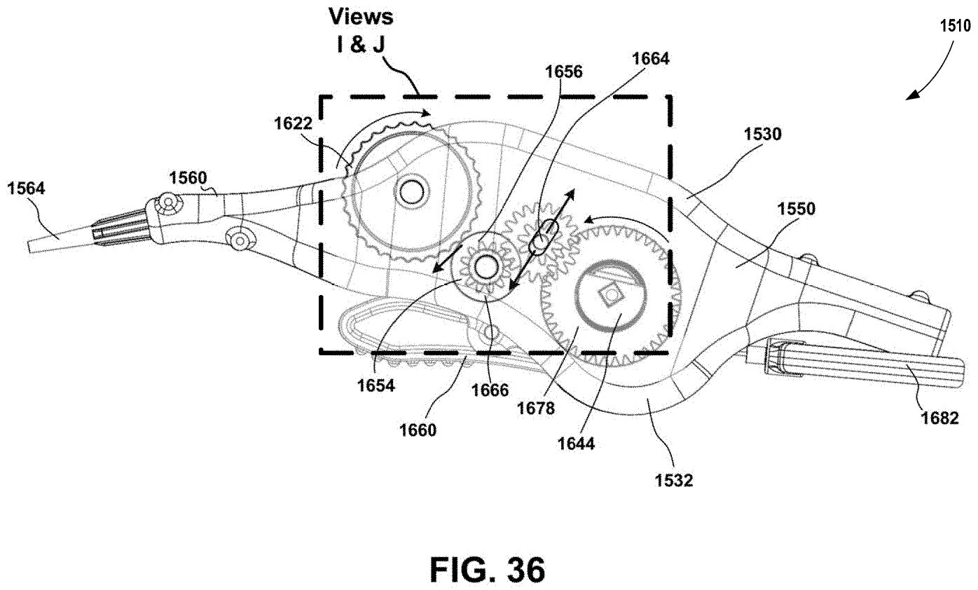

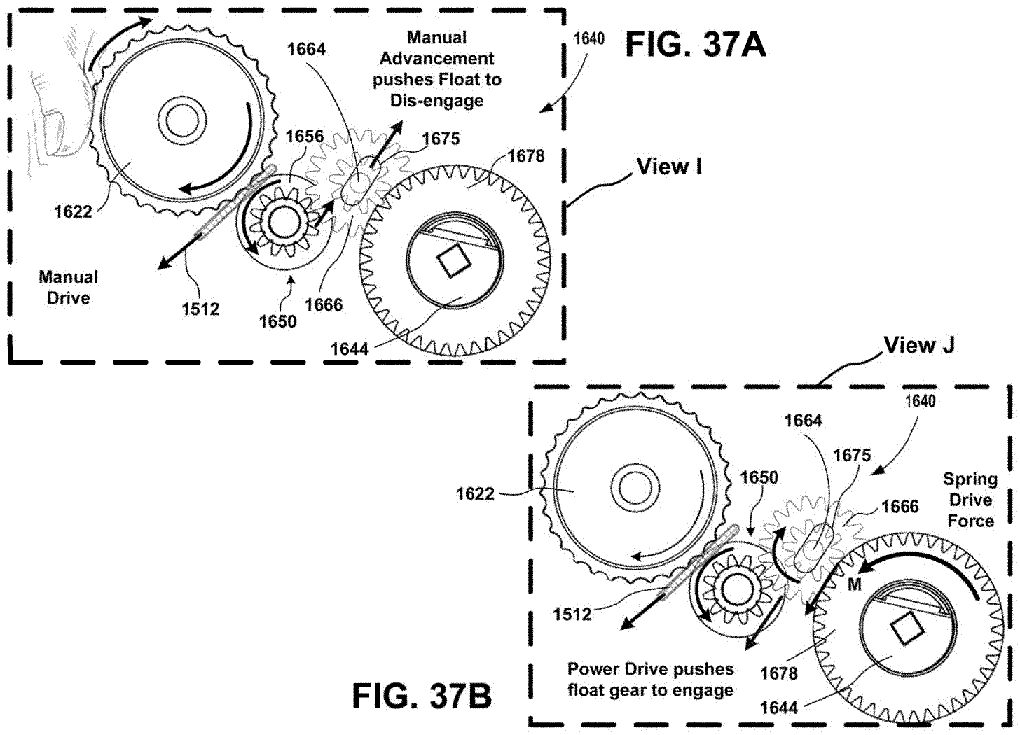

Referring now to FIGS. 36-37B, further details of the manual drive 1620 and the power drive 1640 as drive components of the integrated advancement drive 1630 are shown, as well as aspects and features pertaining to a transmission switch 1663 configured to automatically move between a first position D and a second position E. The transmission switch is configured to automatically move to the first position D when the user exerts manual control via thumbwheel 1622. The first position D de-couples the power drive 1640 from its driving connection with the automated or powered drive roller 1654 to prevent drive force inadvertently being applied by the power drive 1640 on the guide wire 1512 while the user applying fine-tuning user-controlled advancement of the guide wire. The transmission switch is further configured to automatically engage or connect the power driver 1640 to the powered drive roller 1654 when the user actuates the actuator to transmit powered drive force to the guide wire 1512. The transmission switch 1663 generally includes a floater gear 1666, a floater gear shaft 1664, and a pair of guide slots 1675 configured to support opposite ends of the floater gear shaft. The pair of corresponding guide slots 1675 are formed through the left cover 1532 and right cover 1550. The opposing slots support opposite end portions of floater gear shaft 1664 such that the floater gear shaft extends laterally between left and right sides of the guide wire housing 1530 across the width of the guide wire advancer 1510. The floater gear shaft is supported within the opposing guide slots 1675 to allow rotation of floater gear shaft 1664 and sliding translation of the shaft along the slot.

Referring now to FIG. 37A, operation of transmission switch 1663 is shown while the manual drive 1620 of the integrated combination drive 1618 is operative, as well as operations of the manual drive. Guide wire advancer 1510 is configured such that the user applies control movements to the manual control of the thumbwheel 1622 using the thumb of single hand 1518 via thumb movements rearward or in a proximal direction, which rotates the exposed control portion rearward or in a proximal direction. An outer surface portion 1621 of thumbwheel 1622 can be configured to have with a knurled, abrasive, tactile, contoured or other type of surface texture or features providing high frictional engagement, such as with the user's thumb of single hand 1518 and/or for driving engagement of the guide wire 1512. A manual drive portion 1616 of outer surface portion 1621 extends into the guide wire channel 1520 and drivingly engages a side portion of the guide wire 1512 disposed within the nip 1632 to transmit a manual drive force applied by the user to the guide wire. When the manual drive portion 1616 is applying a manual force to the guide wire, automated drive roller 1656 acts as a driven roller and correspondingly rotates in an opposite direction as thumbwheel 1622. Drive gear 1654 is attached to drive roller 1656 and, thus, correspondingly rotates along with the drive roller as shown in FIG. 15C. Mesh gear teeth disposed along drive gear 1654 engage corresponding mesh gear teeth of floater gear 1666 when the floater gear is proximate to or in engagement with the drive gear 1654 and push the floater gear 1666 along guide slots 1675 while rotating as shown responsive to application of a manual drive force. As such, floater gear 1666 automatically slides into the dis-engagement position D, which de-couples the pulley gear 1678 from engagement with drive gear 1654.

Referring now to FIG. 37B, operation of transmission switch 1663 is shown while the power drive 1640 of the integrated combination drive 1618 is operative, as well as further operations of the power drive. Guide wire advancer 1510 is configured such that the user can actuate actuator 1660 as discussed above to release potential energy stored by flat coil spring 1646 such that a moment M applied to pulley gear 1678 rotates the pulley gear in the direction shown. As discussed above along with FIGS. 34 and 35, gear teeth disposed along the pulley gear engage corresponding mesh teeth of floater gear 1666, which applies rotational force to rotate and translate the floater gear along guide slot 1675 into an engagement position E, in which the mesh gear teeth of the floater gear drivingly engage mesh gear teeth of drive gear 1654 to drive rotation of the drive gear in the drive direction shown. Rotation of drive gear 1654 as shown correspondingly rotates drive roller 1656 that is attached to the drive gear and exerts power drive force upon guide wire 1512 disposed within the nip as discussed above along with FIGS. 34 and 35. Thus, floater gear 1666 automatically slides into the engagement position E, which couples pulley gear 1678 into driving engagement with drive gear 1654 when the power drive is actuated. After potential energy stored in flat coil spring 1646 has been expended, the user can apply manual control via the thumbwheel and again disengage the floater as described above in FIG. 37.

### Example Handheld Manual Advancer Arrangements

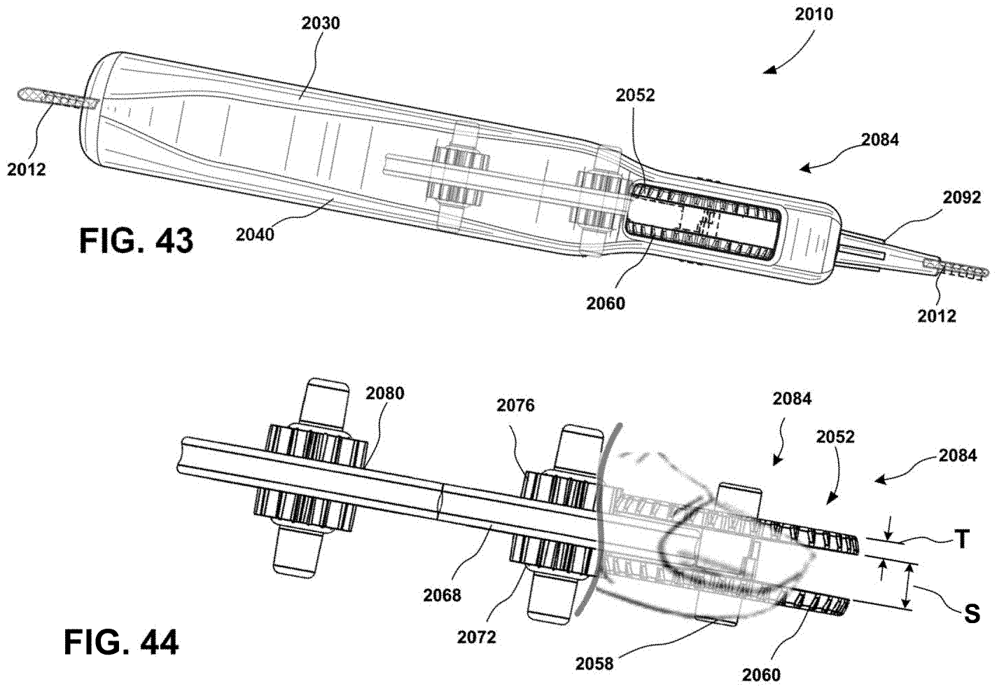

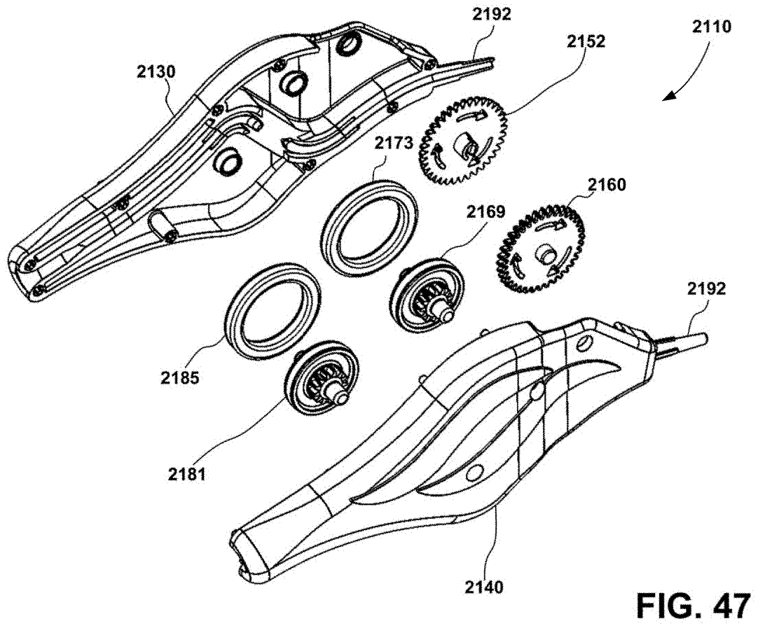

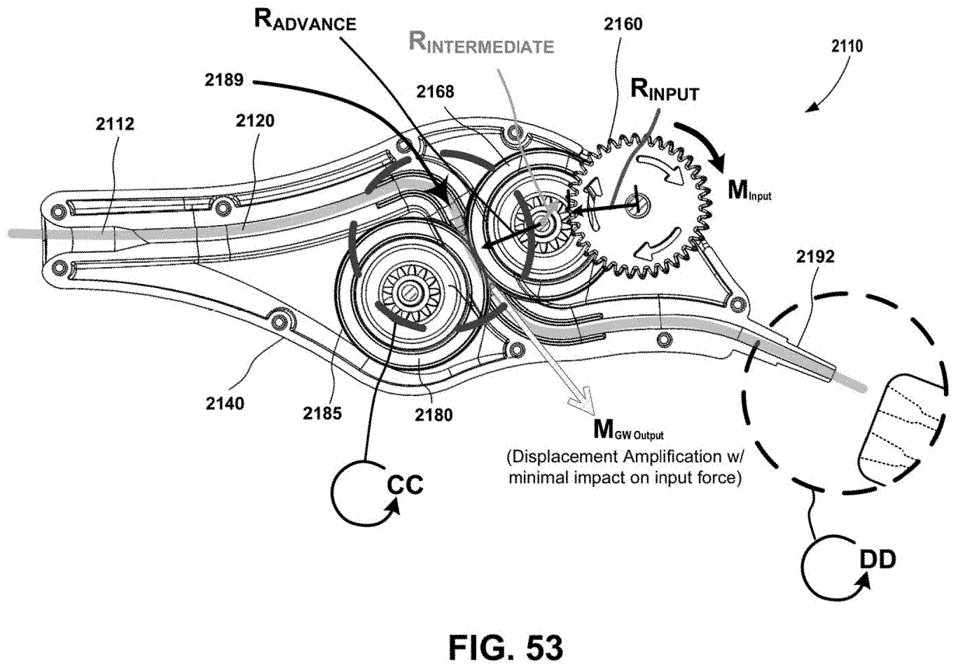

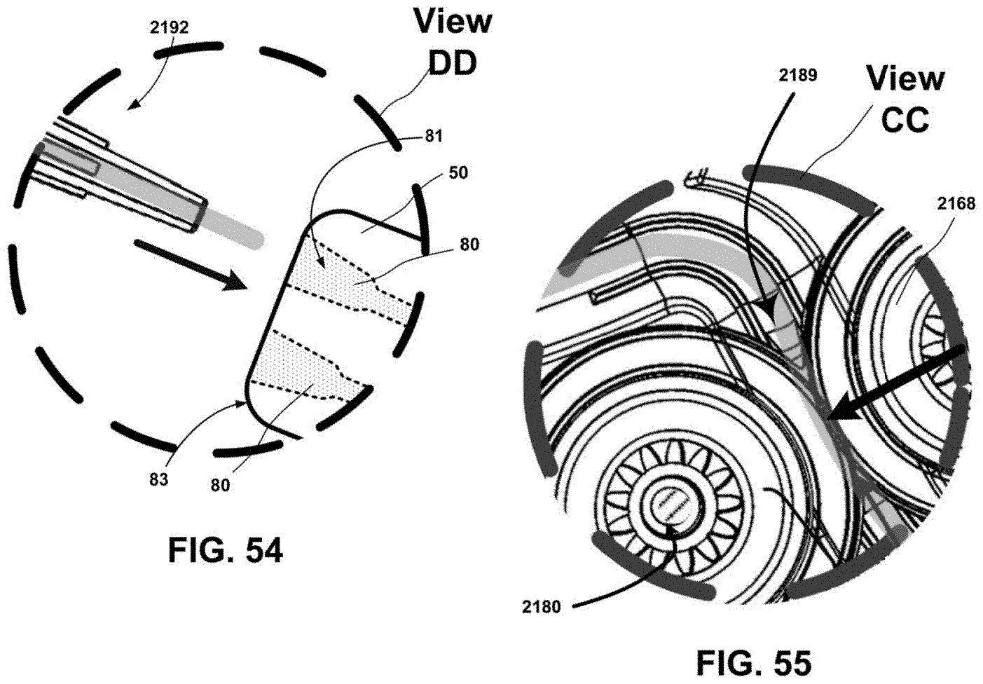

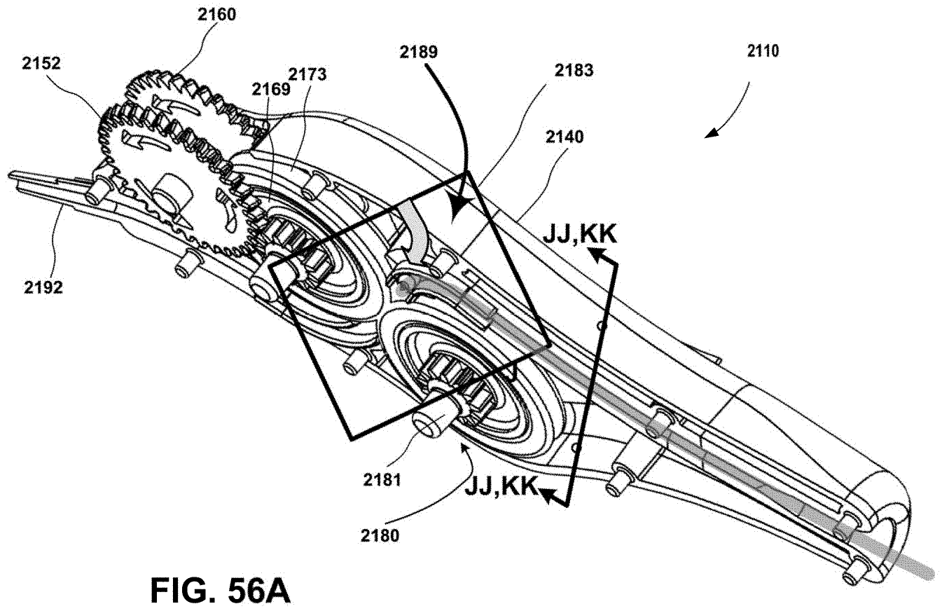

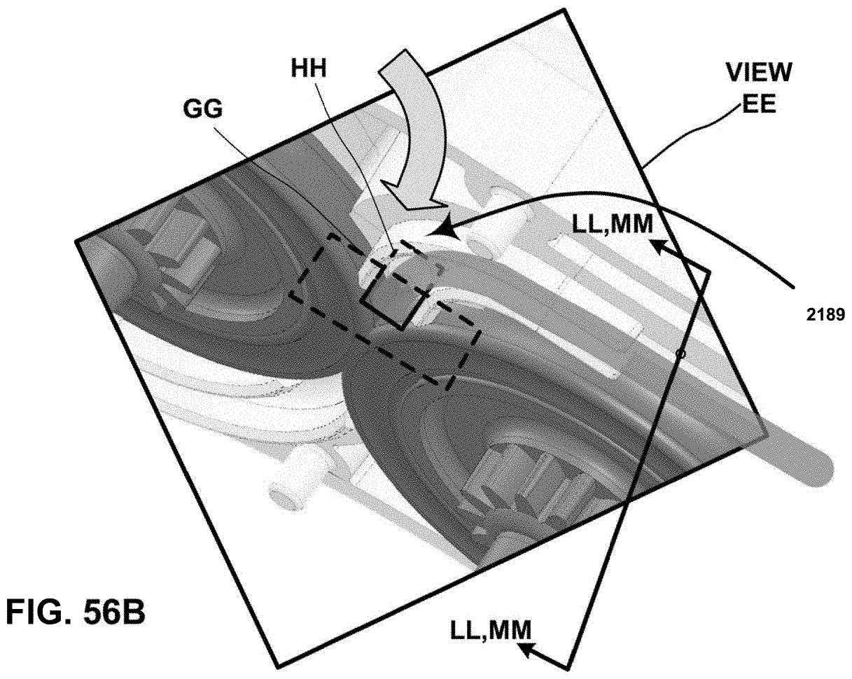

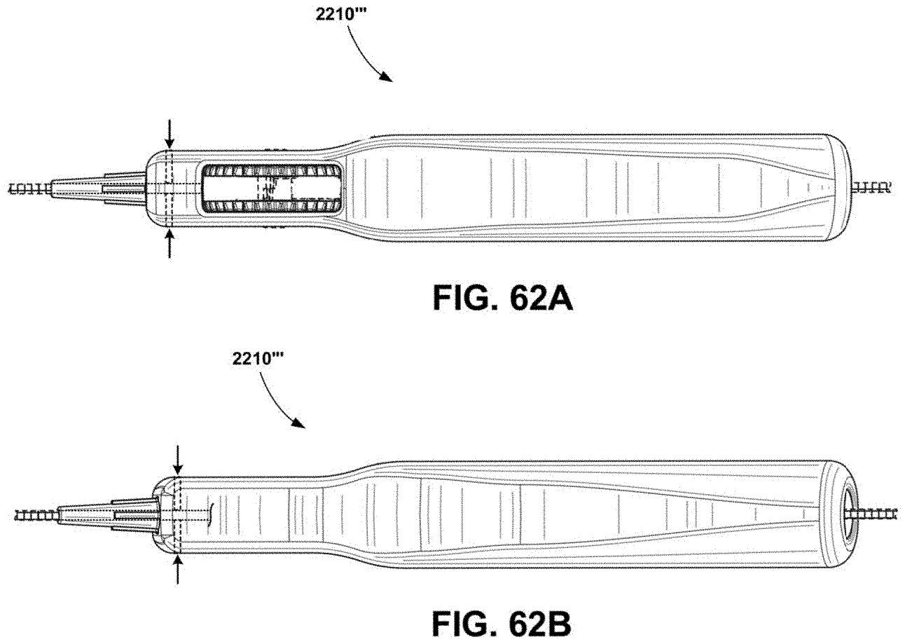

Referring generally to FIGS. 38A-62B, example arrangements of compact, ergonomic, and manually driven elongate device advancers are shown, which are generally identified as advancers 2010, 2110, 2210, 2310 along with several optional variations as denoted by corresponding descriptions and optional reference numbers. For these and other example arrangements for advancers and related features, like numbers refer to like features except as discussed along with each example. Manual advancers 2010 through 2310 shown in FIGS. 38A-62B and other advancers shown and discussed along with additional figures are each arranged for allowing the user hold and readily control the advancer from a single hand for precisely and quickly advancing an elongate device responsive to manual control movements. The examples are often shown coupled with an elongate device in the form of a guide wire along with a corresponding supply depicted as a sheath coil as an example usage arrangement, but operation and usage of the advancer devices are not so limited. Although not necessarily shown for each example arrangement, it is understood that the example elongate device advancers can be operatively configured with a surgical instrument including the suture placement device 50 discussed above along with FIGS. 3A-3E while the suture device may be disposed within a port and prepared for suture placement procedures. Each of the example elongate device advancers are configured to be held within the single hand of a user and easily controlled to perform elongate device advancement actions to support a surgical device, such as the suture placement device 50 discussed above.

As depicted in FIG. 38A, the elongate device advancers including example advancer 2010 of FIG. 38A can be advantageously arranged such that while the user is holding the advancer and controlling it with the single hand 2018, the user can readily impart translation of the elongate device 2012 for advancement through the elongate device advancer and into and/or through a support surgical device 50 or another desired path. The advancer 2010 can be arranged to provide amplified translation of the elongate device 2012 based on the user using the thumb of single hand 2018 and engaging a rotary control input in the form of a thumbwheel or similar controller for engaging a manual drive 2052 and thereby apply a user-exerted rotary input to the thumbwheel for rotating it an input arc distance. Advancer 2010 can further be advantageously arranged and configured to amplify user-exerted input movements applied to the manual input or thumbwheel for translating the elongate device 2012 an advancement distance that is greater than a length of user-exerted input are length. Further, the resultant advancement distance for a corresponding user-exerted control movement can be several times longer than the user-exerted input arclength. Thus, as depicted along with example manual advancer devices, the user can readily grip the advancer and easily control advancement operations without needing to apply numerous repeated advancement movements for imparting significant translation of the elongate device.

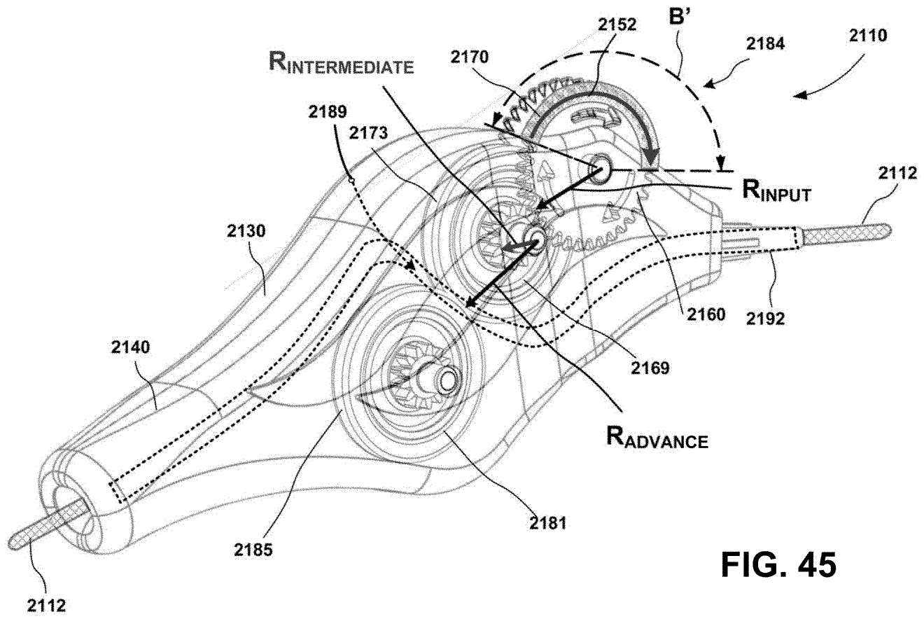

As shown in FIG. 38A along with FIGS. 38B and 38C, elongate device advancer 2010 generally includes an advancer body 2029 formed from a left housing 2030 and right housing 2040 attached to each other in an opposing relationship, a manual control 2084, and an internal manual drive. The advancer body 2029 defines an elongate device pathway that will be discussed in greater detail along with FIG. 45, which can advance the elongate device 2012 therethrough from a first end portion 2023 to a second end portion 2025 of the advancer body while the advancer body is held in a grip of a single hand 2018 of a user and controllable by the single hand. The first end portion 2023 is configured to couple with the elongate device 2012 to receive a tip portion 2016 of the elongate device therein through an entrance 2022 of the pathway disposed at the first end portion 2023, which extends through the pathway and out an exit 2024 of the pathway disposed at the second end portion 2025. The exit 2024 can include a stylet tip 2018 or similar arrangement for coupling the elongate device advancer in a support arrangement with a surgical device, such as a suture placement device.

### Ergonomic Shape, Grip, Control and Related Features or Customizations

Referring now to FIGS. 38A to 44, various features of elongate device advancer 2010 can cooperate with each other to provide an advancer that is ergonomic, simple to use, comfortable for the user to hold and control in the single hand, intuitive to operate, and highly efficient in operation for surgical support uses. As depicted in FIGS. 38A to 41B, the advancer body 2029 can be shaped with an ergonomic and easy to hold (grip) & control shape, such that the advancer body is readily cradled in the single hand 2018. An inboard lower edge region of the advancer body can be shaped to include a precision-control edge region 2011 (FIG. 38A) at a distal end portion of the advancer body proximate the introducer tip 2092, and a power grip edge region 2013 at a proximal end portion. As discussed in more detail hereafter, the precision-control edge region 2011 and the power grip edge region 2011 can be arranged for the fingers to naturally curve about and cradle the advancer body in the hand along with enhancing grip and device control for the user. A stylet tip 2092 can extend from the distal end of advancer at a comfortable angle with grip portions provided by the upper and lower edge portions, which places the wrist at a comfortable, natural control deviation angle (CD ∠) with a feed pathway into which the elongate device advances.

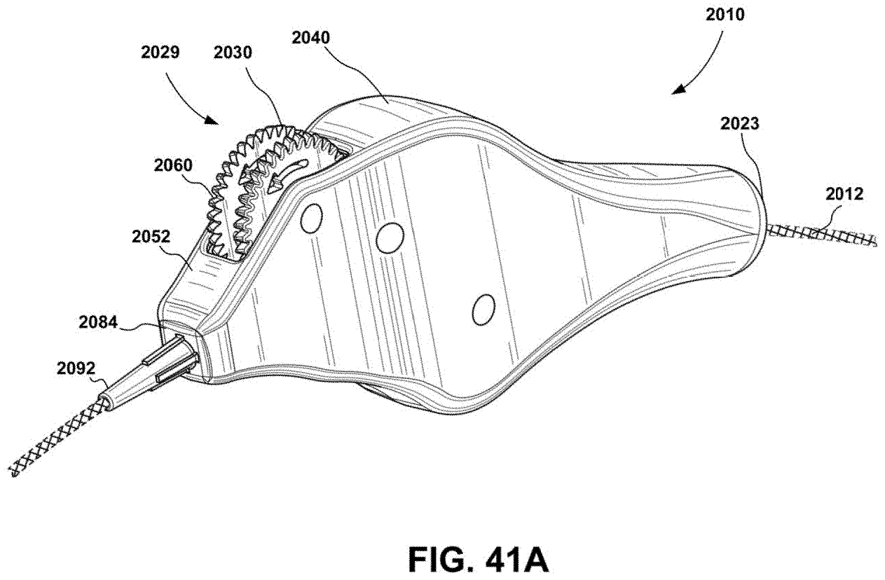

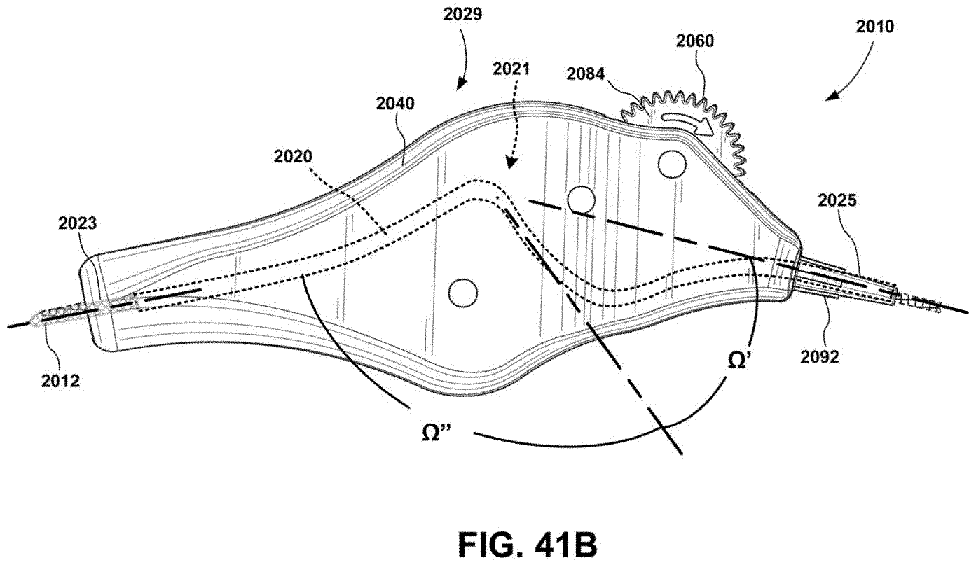

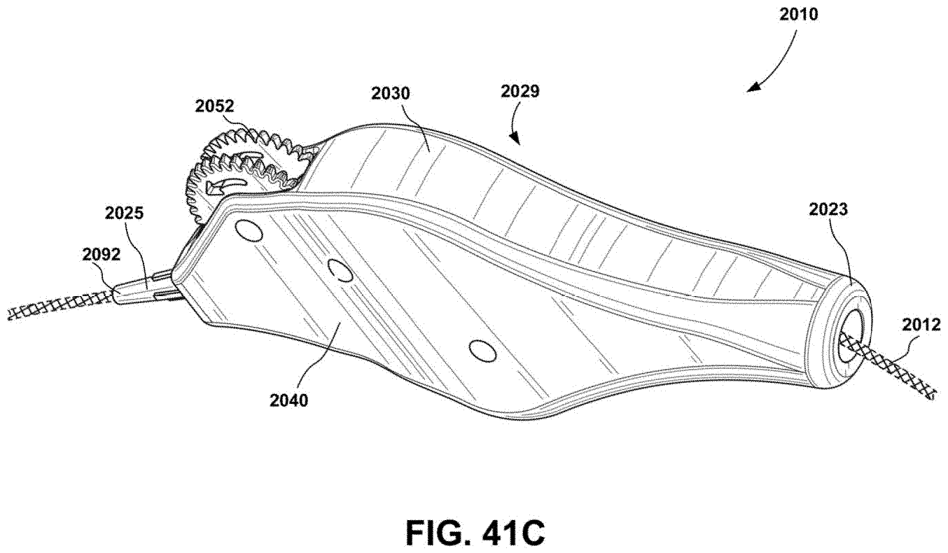

As can be seen in FIG. 41B, the advancer body 2029 can define a pathway 2020 within and through the advancer body 2029 that extends from a first proximal end 2023 to the stylet at the opposite distal end 2025. Although not readily apparent to the user, the pathway can include a significant bend or turn 2021 along a medial portion of the pathway, such that a longitudinal axis of the pathway along the bend portion is angled with respect to a longitudinal axis of the pathway at the stylet rather than being oriented generally parallel with the exit longitudinal axis as is common for conventional advancer devices. Thus, as shown in FIG. 41B, a longitudinal axis of the pathway along a medial portion of the pathway can form an acute tip deviation angle, Ω′, with respect to the stylet longitudinal axis at the tip and a transverse inlet deviation angle, Ω″, with respect to the inlet longitudinal axis at the proximal end portion. The acute tip deviation angle can be relatively small, such as about ten (10) degrees or more, as well as relatively large, such as about sixty (60) degrees or more, and the transverse inlet deviation angle, Ω″, can be generally transverse with respect to the inlet longitudinal axis, such as between about sixty (60) to about one hundred twenty (120) degrees or about ninety (90) degrees.

The example advancer body 2029 depicted in FIG. 41B can include a pathway 2020 defined therein having a medial bend 2021 arrangement, which can cooperate with various other aspects and features discussed herein to provide a compact, small form factor advancer that can easily be held by a single hand of the user and readily control by the user's thumb. Rather than maintaining a generally straight, elongate pathway oriented generally colinear with a length of the advancer may be implemented with conventional advancers, pathway 2020 can include a bend 2021 formed within a medial portion of the advancer 2010, which can provide various advantages and benefits for the advancer arrangement, such as pertaining to ergonomics, operational benefits, and/or efficient and intuitive operability. For instance, such an arrangement and pathway can cooperate well with ergonomic grip features and aspects related to comfort for holding and manipulating the advancer (e.g., wrist angle including minimizing ulnar deviation of the user) as discussed above.

Further, such an arrangement can enable arrangements that can provide beneficial operational benefits, such as amplified displacement of the elongate enclosure during use, powerful grip features, constant precise displacement control of the elongate device, and/or force amplification for driving displacement, which are described in greater detail below. In addition, such an arrangement can better enable intuitive controls for operating the advancer including orientation and location of the stylet at the distal end 2025, and forward thumb roll operation toward an advancement direction of the stylet for the manual input/thumbwheel 2084. Further, schematic representations of advancer 2020 with respect to a user's hand 2018 included with FIGS. 39A to 39E show comparative sizing, placement and orientation of the advancer during usage with a typical (e.g., 5^{th} to 95^{th} percentile adult hand), for which compact size and simple control, handling benefits and features of the advancer can be provided based, in part, on shaping of the pathway 2020 through the advancer as described herein.

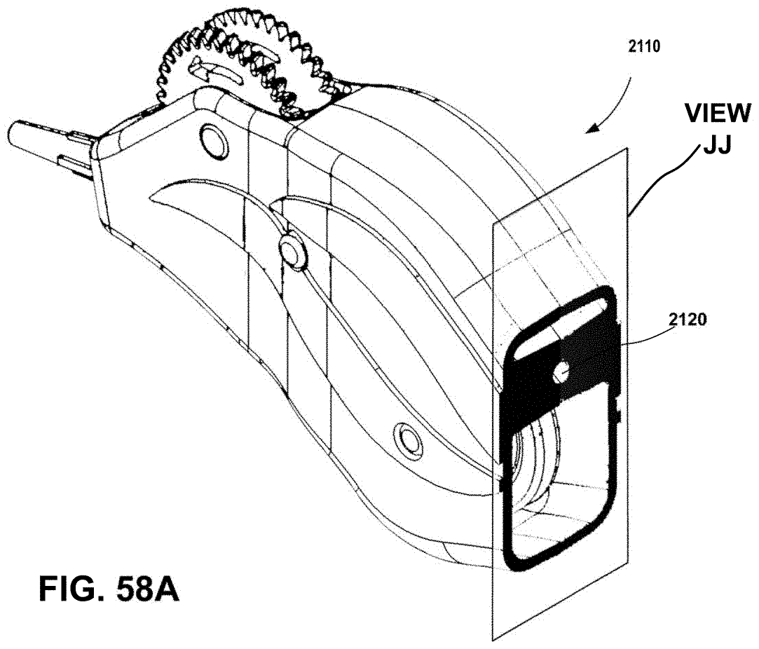

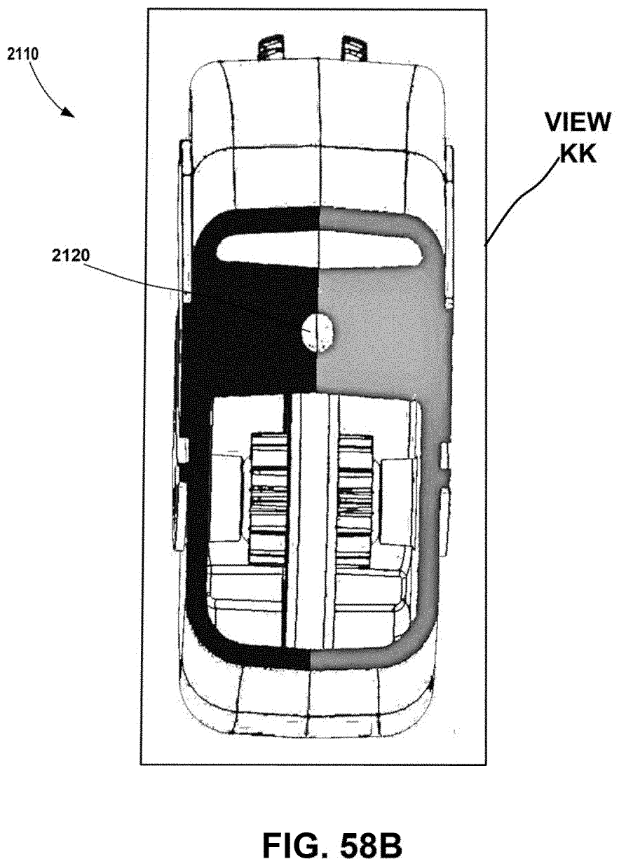

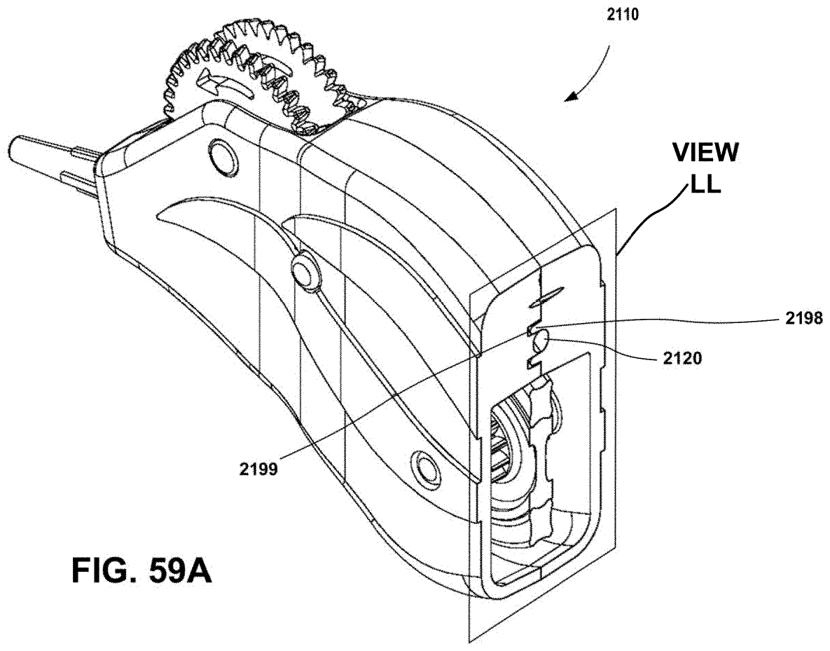

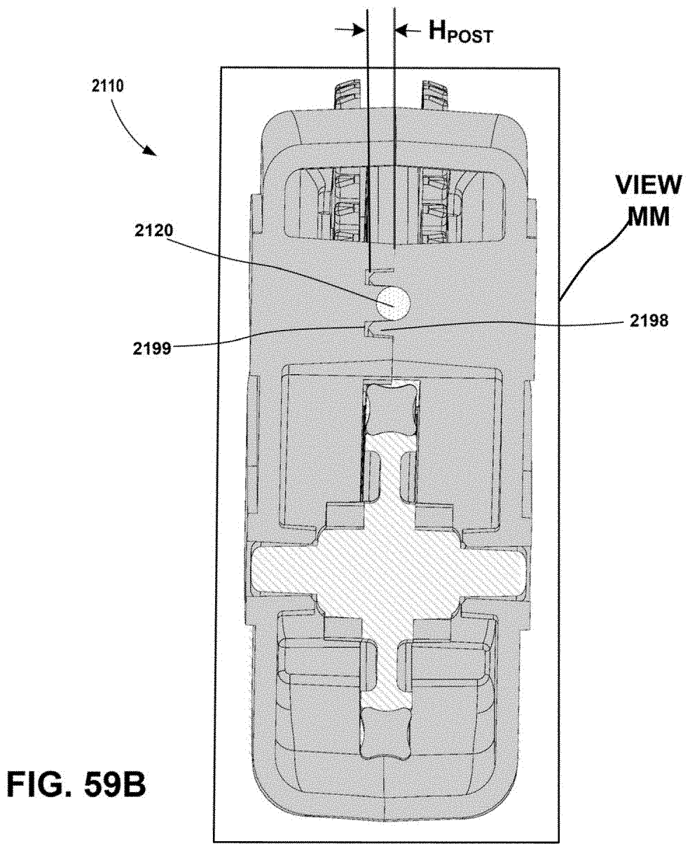

As can be seen in FIGS. 38A to 398, the advancer body can be formed via a mated housing pair including a left housing 1130 and a right housing 1140, which can be attached to each other along opposing, mating surfaces and joined to each other via an adhesive, ultrasonic weld and the like. Each of the left and right housings can be formed from a rigid, lightweight medical grade thermoplastic or similar material, and formed via known methods such as injection molding. As discussed in greater detail below along with FIGS. 58A to 59B, the housings can also include various mating features for providing enhanced structural stability along with other benefits. For instance, mating male protrusions and/or female recesses formed at opposing, corresponding or mating portions of the housings can provide benefits for the arrangement, such as reinforcing or enhancing the pathway defined through the advancer body, providing rotational supports and other support features for internal components, defining geometric features for providing structural stability or reinforcement, and/or providing aesthetic or visual features and cues indicating intuitive usage and operations of the advancer device.

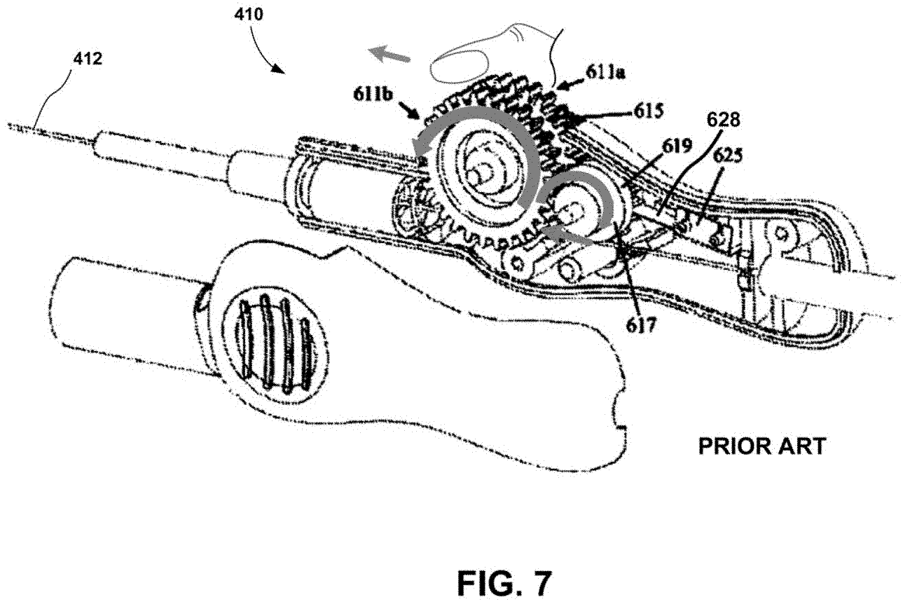

As further depicted in FIG. 39A, the stylet tip 2092 can include features for enhancing interface connections with the tip of the advancer, such size, taper, tubing engagement ribs, and/or other features for allowing ready connection of stylet tip 2092 with flexible tubing 2093 or similar type interface and retaining the same during usage. Further, usage of a flexible interface 2093 as a bridge-type connector between the stylet tip 2092 and a supported medical device, such as provided at the distal tip of the advancer body 2092 for coupling with the suture device 80 or other medical device as discussed above along with FIG. 7 for advancing the elongate body into the suture device as it exits the advancer for representing an example usage of the advancer. In particular, usage of flexible tubing 2093 or similar bridge interface can further enhance user flexibility, controllability, and user-comfort options provided via advancer 2010 via the user and hand 2018 having greater flexibility for the particular orientation and positioning of the advancer 2010 with respect to the medical device 50 during use.

It is understood that attachments can be provided for various types of connections with the stylet 1192 and/or the stylet can be replaced with another adaptor device for providing an appropriate coupling or connection with a different type of surgical device as desired. For instance, as depicted in FIG. 8B, the stylet tip 2092 can be connected with the advancer body using a removable or replaceable connection, such as a screw type connection shown, which can allow various arrangements, types, sizes and related customized stylet tip arrangements be used with the advancer for interfacing with a wide variety of medical devices.

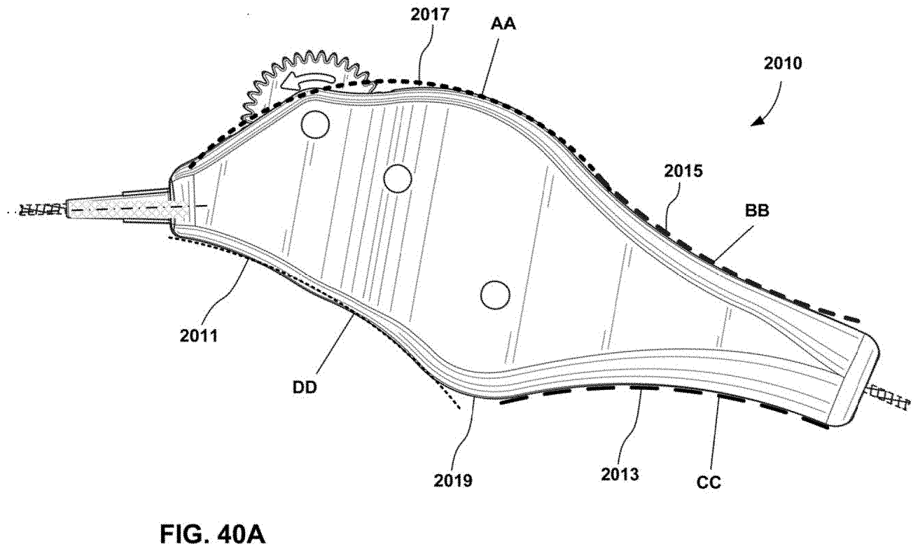

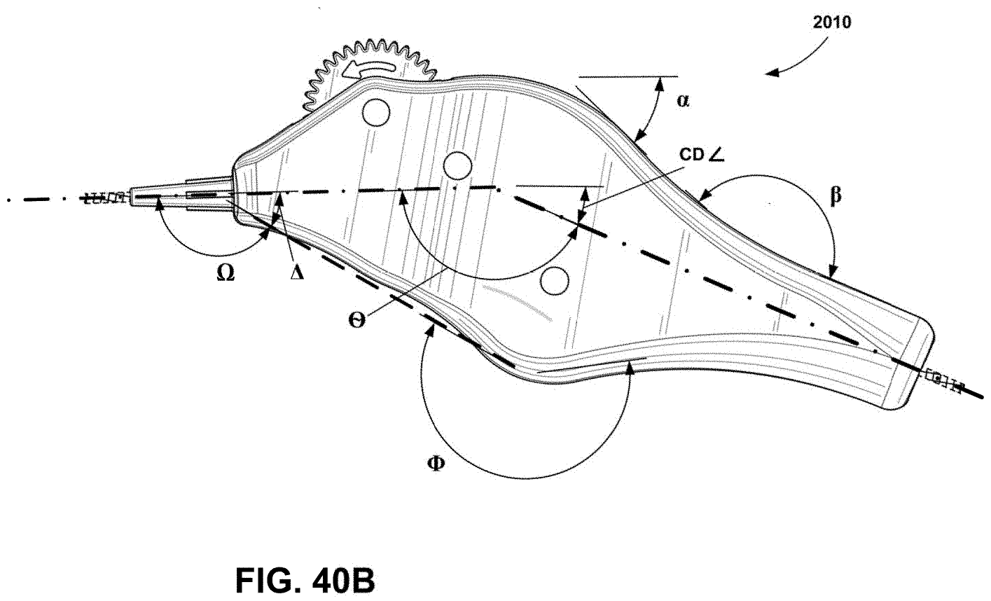

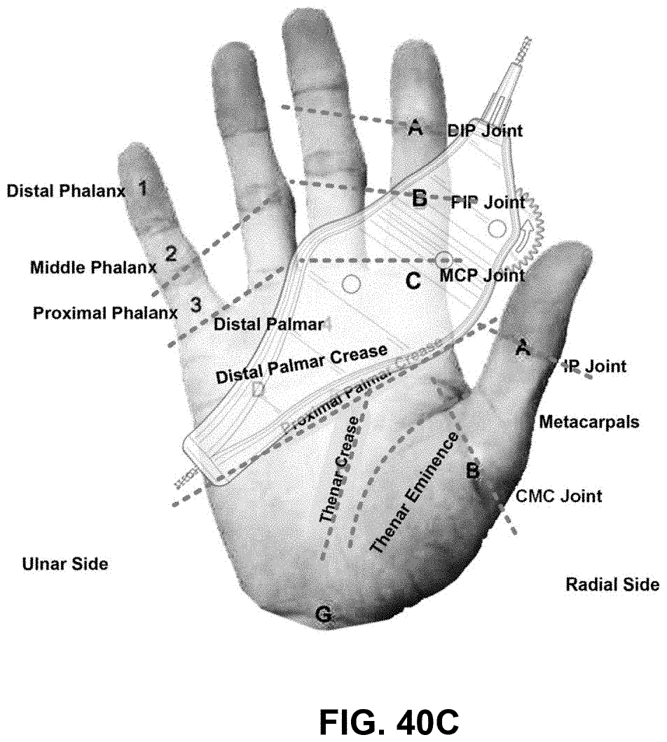

Referring now to FIGS. 40A and 40B, a left plan view of the example advancer 2010 is shown with example edge regions identified as discussed above along with FIG. 38A, which can act as ergonomic shape features that can support, cooperate with, and enhance grip, user control, handling and other grip, comfort and control features that can be incorporated with example elongate device advancers described herein. As noted above along with discussing FIG. 38A, the precision-control edge region 2011 and the power grip edge region 2011 for example advancer 2010 can be arranged according to ergonomic principles and anthropomorphic data for adult human hands with respect to for grip, handhold comfort, usage, control and related performance features. For example, size, shape, grip, comfort, ergonomic principles and other features and parameters for advancer arrangements including, for instance, advancer 2010 can rely on data for typical hand anatomy, ranges of motion and flexibility, grip characteristics, hand contours, curvature and natural positions in a relaxed state based on anthropomorphic data for adult hands falling within, for example, 5^{th} to 95^{th} percentiles (male and female).

Various arrangement aspects, features and/or customizations based this type of data can provide various benefits and operational advances pertaining to example elongate device advancers described along with examples depicted and discussed herein, such as enhanced grip features, improved usability, long-term retention within the user's hand, and extended effective operability and controllability for the user while held in the hand. For instance, representations shown in FIG. 40A for the precision control edge region 2011 and the power grip edge region 2013 include a generally concave shape or curvature DD formed along precision control edge region 2011 and another generally concave shaped curvature CC formed along the power control edge region 2013, which are separated from each other by sharp angle or bend 2019 separating each of the concave-shaped edge regions CC and DD from each other. In addition, a proximal edge region can be defined along an opposite upper edge region of the advancer to form a grip contact surface 2015, which can also define a gentle concave curved shape BB oriented opposite from the lower concave shape CC. Further, a distal edge region 2017 can define a convex curvature AA along an upper distal end portion of the advancer, which can define a thumbwheel access surface 2017 along an upper edge region of the advancer, through which a manual control 2084 can extend such that the manual control is partially embedded within the advancer body extending upward through the thumbwheel access surface 2017.

The arrangements, orientations with respect to each other, and curvatures for AA, BB, CC and DD including concave or convex shapes correspond with general shape and size characteristics of the advancer 2011 with respect to the user's palm and hand 2018 for anthropomorphic data of most adult users (e.g., 5^{th} to 95^{th} percentiles), which synergistically enhances grip strength, ergonomic, control and other related hand-advancer interface relationships. Such advantages and benefits can be enhanced even further based on angular orientations and relationships for palm-advancer interface surfaces of the user for holding, controlling, and operating the advancer 2011. Example angular relationships and orientations for various advancer surfaces and interface portions are shown in FIG. 40B that can cooperate with ergonomic and anthropomorphic data and relationships that can enhance interface features between the user's hand and the advancer, such as ease for gripping and holding the advancer; ergonomic aspects including comfort and long-term ease of access and control for the user and user's hand; and minimizing stress and related impacts to the user, such as avoiding ulnar deviation of the wrist while the user holds and controls the advancer.

Examples of potential beneficial angular relationships and orientations, for instance, can include the following angles that are identified in FIG. 40B:
α—inboard rotation of an upper edge region from the thumbwheel access surface 2017 for an acute angle α, and/or angular rotation for one hundred eighty (180) degrees plus the acute angle angle α with respect to the longitudinal axis at the stylet tip;
CD ∠—Counter Deviation Angle, which is generally an acute angle of deviation representing an inboard offset orientation angle of the input longitudinal axis out a co-planar or co-linear alignment arrangement with the exit longitudinal axis, which can avoid ulnar deviation of the user's wrist for hand 2018 while holding the advancer in a usage arrangement, such as is shown in FIG. 38A;

### Manual Drive/Thumbwheel Input and User Engagement

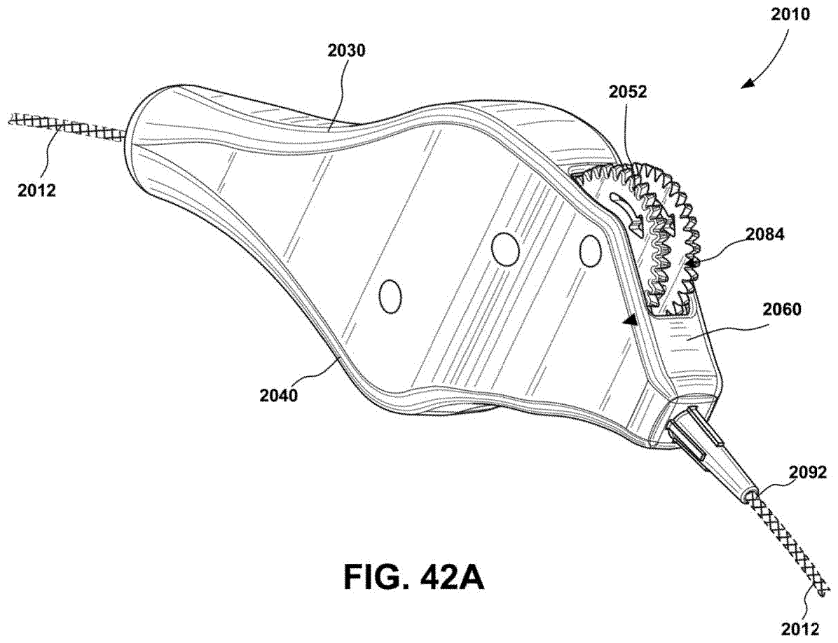

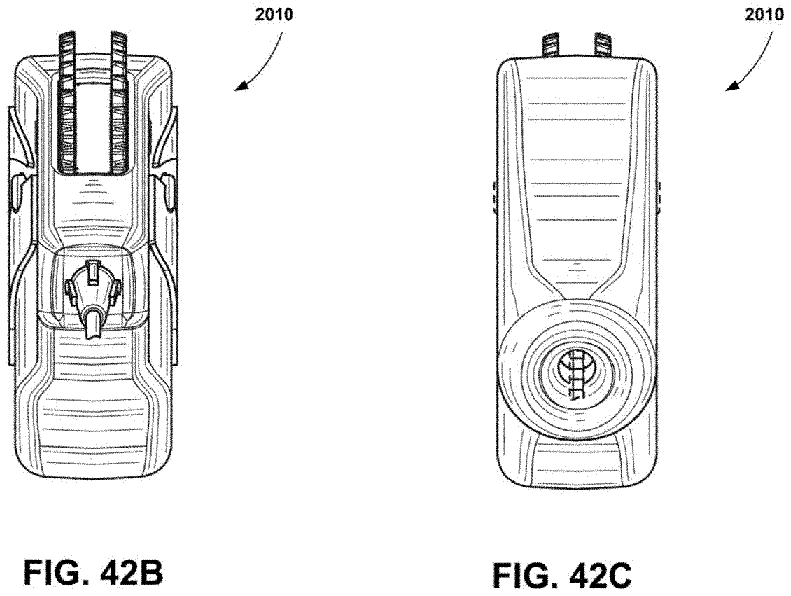

With particular reference now to FIGS. 42A-45, as well as FIGS. 37A-59B in general, the manual control 1184 is generally formed as a thumbwheel type mechanism disposed proximate the distal end portion and stylet tip 1192, which can be located along an upper portion of the advancer body that is arranged to support the user's thumb and thereby allow for natural operational control. The manual control can be arranged as shown and indicated for receiving rotary controls from the user including being arranged such that forward rotation toward the stylet tip imparts translation of the elongated device in a corresponding advancement direction for exiting the stylet tip. As show, visible indicia can be included on or proximate the manual input for guiding intuitive control movements of the user, which can be helpful and important during complex surgical procedures for properly operating the advancer. The manual control 1184 can be arranged for rotation and conveniently disposed along an upper portion of the advancer body configured for ready reception of user-exerted rotary input. Further, the manual control in a thumbwheel type arrangement can be partially embedded within the enclosure body in an arrangement for exposing a significant upper exterior portion of the rotary input outside of the advancer body, which can be accessible to the user and arranged for simple application of user-exerted control inputs thereto from the thumb of the single hand.

As can be seen in FIG. 45, the manual control can receive a user input arc for movement of the manual control based on the exposed rotary angle, β, of the manual control exposed and accessible for user input and control, which can be about ninety (90) degrees to over one hundred eighty degrees (180), such as for an expanded input arc arrangement having a raised pivot for the manual control providing ready thumbwheel access for the user. In other arrangements, such as represented in the example of FIG. 45, the exposed rotary angle can be about one hundred twenty (120) to almost one hundred eighty (180) degrees. In other arrangements, the exposed rotary angle can be about one hundred thirty (130) to one hundred sixty (160) degrees. In such arrangements, an extended user-exerted control arc can be applied for the span of the exposed rotary angle. The exposed rotary angle can exceed one hundred eighty degrees. However, such arrangements can impair quick and easy access options for the user to impart control movement to the manual input.

As best seen in FIG. 44, the manual control 1184 can include a left drive wheel 1152 and a right drive wheel 1160 disposed adjacent to each other and axially aligned along a thumbwheel shaft 1158 for joint rotation with each other about the thumbwheel shaft. The left and right drive wheels can be spaced apart or offset from each other by drive space, S. Further, one or both of the left and right drive wheels 1152, 1160 can be formed as a thin drive wheel having a thickness, T, that are each less than the drive space, S. Further, the thickness, T, of both drive wheels together can be less than the drive space. S, for providing enhanced engagement with the thumb. The combined thicknesses, T, and drive space, S, can be provided and arranged to be less than a typical thumb width.

Upon initial receipt of a user-exerted control movement, an engagement surface of the thumb of the single hand can encounter the manual control at an engagement angle that can be close to ninety degrees if the thumb rolls forward from the top edge portion of the advancer body. If the initial engagement angle is ninety degrees or close to ninety degrees, such as about sixty to ninety degrees, the engagement angle can provide a high frictional engagement according to the angle, as well as the force applied and the coefficient of friction for the engagement. It can be helpful when initiating translation of an elongate device from a rest position to have high engagement at the beginning of a manual control movement for assisting the elongate device to start translating from a rest position, which can be difficult for slippery surgical environments and for engagement with the thumb occurring though gloves.

An arrangement for the manual control having a pair of thin drive wheels spaced apart from each other by a drive space, S, and having a thickness, T, less than the drive space can enhance user engagement with the manual control. The pair of spaced apart drive wheels maintains and can even enhance a stable engagement region for the thumb of the single hand for avoiding slippage of the thumb to the left or right of the pair of drive wheels during engagement attempts, such that the thumb can bridge across the drive space and further partially fit within the drive space for effectively engaging the manual control and maintaining stable contact even for a low friction environment. Further, each of the pair of thin drive wheels is configured to concentrate engagement forces applied thereto by the thumb along with concentrating the reactionary engagement force applied to the thumb from each of the edge portions of the drive wheels. As such, the arrangement of a spaced apart, thin pair of drive wheels provides an enhanced engagement arrangement and stable engagement contact with the user's thumb for receiving the user-exerted input movement without slippage or similar losses.

As can further be seen in FIG. 45, a corresponding edge portion of each of the drive wheels can define a series of peaks and valleys, which can further enhance engagement with the user's thumb. Each of the peaks contacted by the user's thumb while applying the user-exerted control movement can concentrate the engagement forces and reactionary forces at the tips of the peaks contacting the user's thumb and further enhance the engagement contact. As such, engagement with the thumb of the single hand can be significantly enhanced to avoid input losses even for low-friction, slippery environments including engagement contact via wet gloves. The example arrangement of advancer 1110 for the manual control 1184 can provide high engagement contact as the user begins to exert a control movement, which input provided can include a user-exerted input art having an input arc length.

As discussed further below, beneficial features can be provided for the elongate device advancer based, in part, on the arrangement of the manual control 1184 including the arrangement of the pair of input wheels 1152, 1160. For example, the series of peaks and valleys for each input wheel can be formed as gear teeth, and each input wheel can form a drive gear having a radius, R_{CONTROL}. Further, each of the drive wheels 1152, 1160 can be arranged in a driving relationship with a lateral driven gear 1172, 1176 disposed at each side of the first drive roller and/or formed as part of the rotary axis for the first drive roller or attached with the input roller 1168 to rotate together about the rotary axis, such that the pair of drive gears 1160, 1152 each engage the first drive roller 1168 at outboard portions engaging the driven gears 1172, 1176 of the first drive roller in a stable bracketed or enveloped relationship. The gear teeth of each of the thin drive wheels can operatively engage corresponding teeth of the lateral intermediate driven gears attached to the first input roller, such that user-exerted movement imparted to the pair of drive wheels imparts movement at each of the driven intermediate gears of the first drive roller.

A matching rotation or movement distance for the mating gear teeth of the first and second input gears 1152, 1160 and movement they impart to the driven gear 1172, 1176 can be an arc length of the first and second input gears 1152, 1160 for a user-exerted movement applied to the first and second input gears. Although rotation of the first and second input gears for the input arc length and corresponding movement of the first and second input gears for the input art length can impart matching movement of the first roller driven gears 1172, 1176 for the input arc length. An intermediate rotary arc can be imparted to the first roller driven gears for the gear-connection movements of the first and second input wheels, which can be a function of the radius, R_{INTERMEDIATE}, for the driven gears. Thus, rotation of the intermediate input gears for an intermediate arc is applied to each intermediate gear in response to the input control wheels receiving a user-exerted control movement for an input arc 1170.

However, the span or rotation angle of the intermediate driven gears 1172,1176 can be a function of the radius, R_{INTERMEDIATE}, for the driven gears. The radius, R_{INTERMEDIATE}, for the driven gears is a smaller radius than the radius, R_{INPUT}, for the first and second input gears 1152, 1160 even though each of the intermediate driven gears rotated the distance of the input arc length. That said, the common rotation distance for the geared connection can identify the intermediate arc of the rotation imparted to the intermediate driven gears, which can be determined from the corresponding radii and a control input drive ratio of the corresponding radii (R_{INPUT}/R_{INTERMEDIATE}). As such, each of intermediate driven gears 1172, 1176 rotate an intermediate arc for the rotation imparted via the gear connections for moving the input arc length according to the control input drive ratio. Thus, each of the intermediate driven gears 1172, 1176 rotate an amplified imparted intermediate arc that is (R_{INPUT}/R_{INTERMEDIATE}) times the rotation of the first and second input gears for the user-exerted input arc 1170.

In some translation amplification arrangements between a drive gear rotating a user-exerted input arc, a control input drive ratio can range from about 2 to 4, such that the intermediate driven gear is rotated an amplified intermediate arc angle that is 2 to 4 times as large as the input arc 1170 exerted by the user. In many arrangements, the control input drive ratio for a translation amplification arrangement can be about 2.5 to 3, such that the intermediate driven gear rotates an intermediate arc angle that is 2.5 to 3 times the input arc angle, which can significantly enhance the utility, efficiency and effectiveness of such an elongate device advancer based on a first amplification of the user input occurring between the first and second input gears and the intermediate driven gears.

The initial amplification discussed above for an operative rotary engagement relationship between the first and second input gears and the intermediate driven gears can be permitted and supported via a shape of the pathway through the advancer body 1129 as shown in FIG. 15 and discussed above along with shape and arrangement features of the advancer body. Namely, a bend or curve along a medial portion of the pathway defined within the advancer device can enable and enhance operations for a translation amplifying advancer arrangement, as well as for force amplifying arrangements as will be discussed in greater detail hereafter. Potential amplification and/or mechanical advantage options for advancer drive mechanisms can be enabled and enhanced in accordance with bend or configuration options for the pathway as described below along with operations of the drive rollers and nip.

Manual Input Translation Amplification and/or Force Amplification

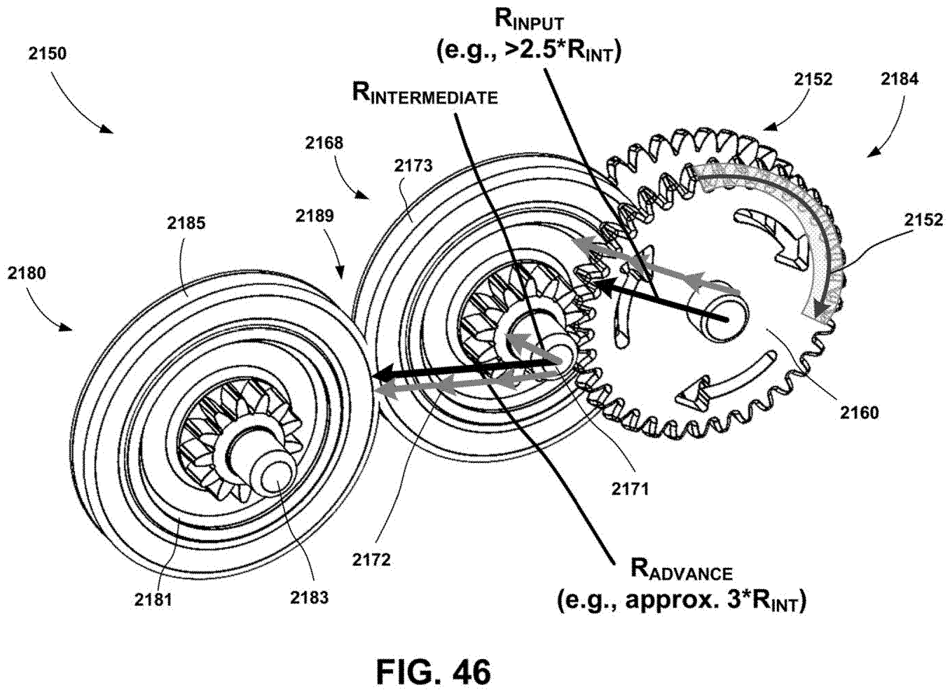

Referring now to FIGS. 46, 47, 52, 53 and 55, components of the manual drive 2150 along with components of the manual control 2184 are generally shown for the elongated enclosure advancer 2110 showing operative arrangements between the components. The manual drive generally includes a first drive roller 2168, a second drive roller 2180, and a nip 2189. The first drive roller 2168 is rotatably coupled with the advancer body via a shaft 2171 and includes an outer engagement surface 2173 and a first driven gear 2172. The shaft 2171 is disposed through a central portion of the first drive roller and includes end portions that extend outward on both sides for rotatable attachment to the advancer body 2129. A first driven gear 2172 is attached to the shaft 2171 and is disposed through the central portion of the first drive roller with the shaft, such that the first driven gear extends laterally beyond the first drive roller and forms gear teeth disposed about the shaft on each lateral side of the first drive roller. The first driven gear 2172, the first drive roller 2168, and the outer engagement surface are attached to each other such that the first driven gear, the first drive roller and the outer engagement surface rotate together about the shaft. A drive portion of the first drive roller includes the engagement surface 2173, which is configured to extend into the pathway and engage side portions of the elongate device 2110 within the pathway for driving translation of the elongate device.

The second drive roller 2180 is rotatably coupled with the advancer body via a shaft 2183 and includes an outer engagement surface 2185. The shaft 2181 is disposed through a central portion of the second drive roller and includes end portions that extend outward on both sides for rotatable attachment to the advancer body 2129. The second drive roller 2168 and the outer engagement surface 2185 are attached to each other such that the first drive roller and the outer engagement surface rotate together about the shaft 2183. A drive portion of the second drive roller includes the engagement surface 2185, which is configured to extend into the pathway opposite the first drive roller engagement surface 2173 and engage side portions of the elongate device 2110 within the pathway opposite the first drive roller engagement surface and apply an advancement drive force to the elongate device.

The nip 2189 is defined between the engagement surfaces 2173, 2185 of the first and second drive rollers extending into the pathway in an opposed arrangement. The opposing engagement surfaces 2173 and 2185 are configured to grip the elongate device between the engagement surfaces and cooperate as a pair of drive rollers to translate the elongate device along the pathway between the first and second end portions. Further, the opposing engagement surfaces 2173 and 2185 are arranged and operatively connected to each other and with the manual control 2184 to impart amplified translation movement to the elongate device in response to the manual control receiving a user-exerted rotary movement as discussed above along with the manual control. A translation distance of the amplified translation movement is greater than an arc length of the manual user-exerted input arc applied to the manual control 2184.

Further to the discussion above of the previous section pertaining to the manual control 2184, the manual control converts the initial input force and input movement applied as the user-exerted input arc 2170 to the manual control 2184, such as movement applied to a thumbwheel-type mechanism like the pair of input wheels 2160 and 2162, and does so according to mechanical advantage principles, Newton's Second Law of Rotational Motion and/or conservation of angular momentum principles, as well as preservation of applied moments. Elongate device advancers arrangements according to such aspects and preferences discussed herein along with the present examples can makes beneficial, innovative use of these principles for providing elongate device advancement arrangements that can perform significantly improved, effective and efficient elongate device advancement operations—and do so for manually driven, user-exerted advancement.

The rotational moment exerted by the user for rotating manual control 2184 the input arc length 2170 is transmitted by input wheels 2152 & 2160 to the first roller driven gears, which rotates the same arc length distance as the input arc length via gear contact with the input wheels, but for the intermediate driven gears having a smaller radius and thereby having amplified rotation imparted to the intermediate driven gears 2172, 2176 to rotate an amplified arc angle that is greater than the user-exerted input arc. Because the first roller driven gear 2172 is rotatably attached to the first roller driven gear about the shaft 2171, the first roller driven gear further imparts rotation of the first drive roller 2172 about shaft 2171 for the amplified intermediate arc.

The corresponding amplified rotation of the first input roller 2172 rotates simultaneously with rotation imparted to the first driven gear 2172 (adjoined) for the amplified rotary angle, which permits the first input roller 2172 having a greater radius than its first driven gear 2172 to amplify further the amplified arc imparted to the intermediate driven gear, such that the translation distance applied to the elongate device can be enhanced further. In other words, the manual control 2184 operatively (rotationally) connected to the first input driven gear 2172 performs an initial output (imparted) rotary angle amplification, which the first input roller 2168 can further amplify along with applying the resulting final amplification to the elongate device at the nip. Note also that force amplification can be provided as well according to mechanical advantage principles, such as generally based on the ratio of the output radius of the drive roller 2168 (R_{ADVANCE}) with respect to the input radius of the manual control 2184 (R_{INPUT}) or (R_{ADVANCE}/R_{INPUT}).

For instance, FIG. 46 depicts as an example an arrangement in which the manual input 2184 for each of the pair of thin input wheels 2152 and 2160 have a radius, R_{INPUT}, about which an example user-exerted input arc of about ninety degrees can be applied. Rotation of input wheel 2160 as shown for an input arc of ninety degrees imparts rotation of the first driven gear 2172 according to movement at the gear interface for the input arc length. However, the radius, R_{INPUT}, of the input wheel 2160 is about 2.5 times the radius, R_{INTERMEDIATE}, of the first roller driven gear 2172. As such, the first driven gear 2172 rotates an amplified intermediate arc angle that is about 2.5 times the user-exerted input arc applied to the thumbwheels, which for the example would be about two hundred twenty-five degrees, AND the intermediate driven gear 2172 further rotates the attached first input roller 2168 along with the intermediate driven gear for the increased angular rotation of two hundred twenty-five degrees, but with an extended moment arm.

A radius of the first input roller 2168, R_{ADVANCE}, has a radial length from the common axis (shaft 2171) with the first roller intermediate driven gear, that is about 3 times as long as the radius, R_{INTERMEDIATE}, of the first roller intermediate driven gear 2172. As such, an arc length for the same rotation imparted to the first roller intermediate driven gear 2172 (e.g., two hundred twenty-five degrees) provides to the first input roller 2168 an arc length that is about three times greater than the arc length provided by the same rotation for the intermediate driven gear 2172, which when applied by the first input roller 2168 at a distal engagement end 2173 of the nip drives the elongate device an amplified translation distance for which the first drive roller drives the elongate device, and does so with a moderate to negligible mechanical advantage.

Thus, a length of a user-exerted input arc can be amplified, for example, to drive the elongate device for the present example a length that is about seven and a half times longer than the user-exerted input arc length, and does so via a mechanical advantage/force amplification factor of about 3/2.5 or 1.2 Such large amplifications of user inputs, such as via a thumbwheel, that effectively translate and advance an elongate device, such as a guide wire, during surgical operations can significantly enhance operability of the advancer, overcome many shortcomings and challenges of conventional manual advancer mechanisms, and ultimately enhance surgical procedures and operations involving the same—especially when operable based on about the same amount of input force or even slightly less input force than would be required to physically push translation of the elongate device or directly drive a thumbwheel 2184 of similar size to translate the elongate device via direct contact.

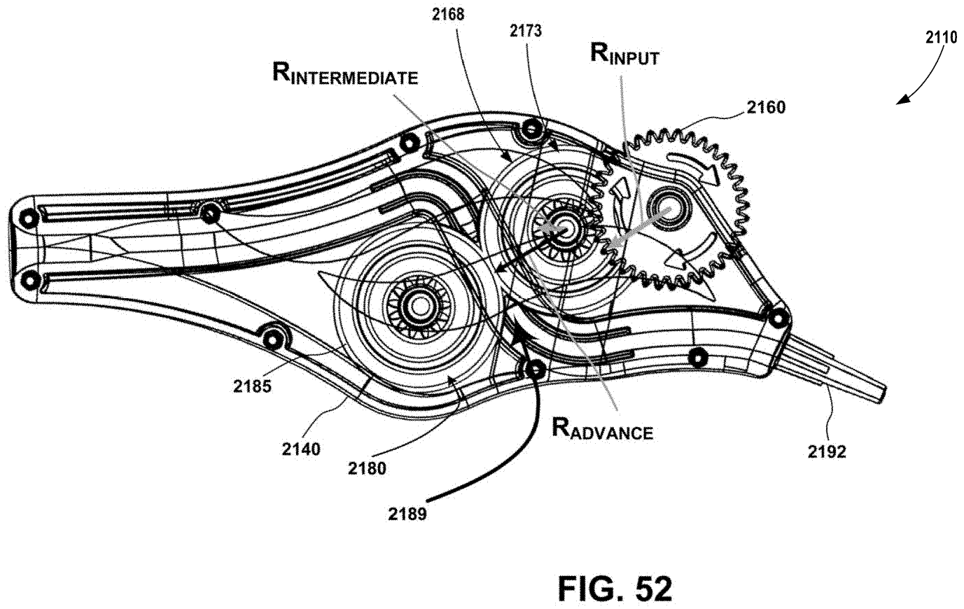

However, drawbacks and challenges for conventional advancer mechanisms include conflicting interests between being able to provide an advancer having a lightweight, easy-to-hold and control, ergonomic advancer arrangement, vs. amplifying advancer or translation movements for significantly reducing required user-exerted inputs for operating known manual advancers. With reference again to FIGS. 52, 53 and 55, the applicants have researched, tested, and identified arrangements for manual elongate device advancers that significantly reduce manual inputs for driving the advancement and translation, as well as providing a wide range of beneficial features including providing compact ergonomic arrangements as discussed above that simple and comfortable to hold, control and use in a single hand. As can be seen in FIGS. 52, 53 and 55, relationships between features such as manual control/input mechanisms, drive and roller arrangements and connections, and other aspects and features discussed along with examples herein can be permitted and supported, at least in part, via innovative and creative pathway arrangements, as well as other inventive mechanisms, aspects and features including drive mechanisms as discussed in further detail below.

### Manual Drive and Gripper-Driver

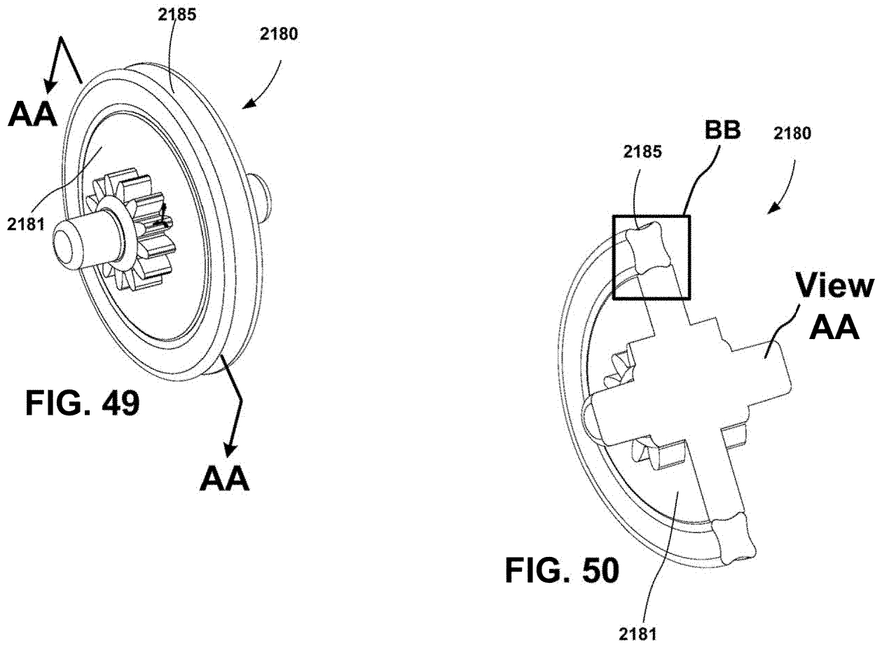

Referring now to FIGS. 49-578, further aspects, features and related innovative and creative concepts pertaining to the example manual drive 2150 discussed herein are shown, which generally involve operations and features pertaining to the nip 2189 and engagement of elongate devices therein. Such features cooperate with various other beneficial aspects and features previously discussed herein, which overall describe well-developed, effective and efficient concepts for configurations of a manual introducer/advancer mechanism for elongate devices that can be used with many different surgical procedures and for various types of elongate devices. Various concepts, aspects and features illustrated along with the examples of the above-listed figures provide further insight and understanding regarding these mechanisms and options for the same.

Referring now to FIGS. 52, 53, 55 and 56B, various views of a nip 2189 are shown as formed from a corresponding set of rollers including first drive roller 2168 having an engagement portion extending into the pathway, and a second drive roller 2180 opposite the first drive roller also having an engagement portion extending into the pathway opposite the first drive roller engagement portion. Each of the engagement portions are configured to rotate with the respective drive roller for each engaging an elongate device therebetween to impart translation forces to the elongate device and translate the elongate device along the pathway. A nip 2189 is formed between the first and second drive rollers and the respective engagement portions. Although referred to as a drive roller, the second drive roller 2180 can be configured as an idler roller such that it does not receive any active drive forces for imparting advancement forces to the elongate device.

However, note that each of the first drive roller 2168 and the second drive roller 2180 generally have the same configuration, such that both rollers are about the same size and have the same support structure and arrangement with the same type of engagement portion disposed thereon as an outer portion of the drive roller. As such, unlike many conventional nips and drive roller arrangements, the elongate device is gripped between the two drive rollers in a matching bilateral arrangement having balanced forces applied to the elongate device at opposite sides of the elongate device. Further, although the second drive roller 2180 can be configured as an idler roller, the second drive roller 2180 is arranged to provide equal and opposite opposing forces or reactionary forces with respect to the first drive roller 2168 against an opposite side of the elongate device. Thus, the nip 2189 and arrangement of drive rollers 2168, 2180 can arranged as a bilateral, balanced opposing forces arrangement against opposite side regions of the elongate device, which can surround the elongate device in a firm, balanced grip about its circumference having a high contact area with the elongate device effectively and efficiently applying translation forces to the elongate device.

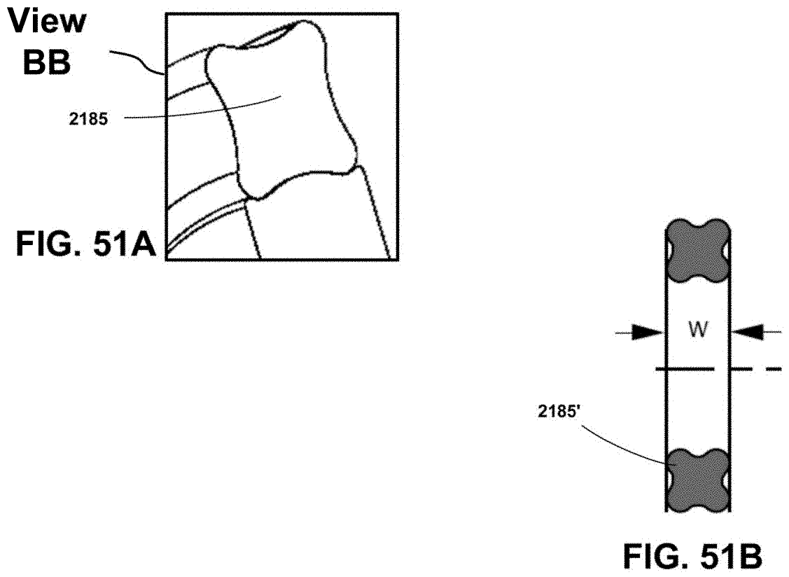

Referring now to FIGS. 49-51B, cross-sectional views of the second drive roller 2180 are shown as representative examples for either of the drive rollers. As shown, second drive roller 2180 includes an inner support frame 2181, which can be formed as a generally rigid support frame. The inner support frame 2181 includes an outer support surface defining either a groove or raised portion along the support surface. The second drive roller 2180 further includes an O-ring configured as a double seal O-ring. The double seal O-ring is one example of a newer family of O-rings that are also known as Quad-Rings or X-Rings, which are each arranged as circular O-rings configured to form a gripping, non-slip relationship with the outer support frame. Further each of the O-rings can be formed from an elastomeric material having a wide range of properties including high compressibility and high frictional contact properties as desired. Each of the O-rings include a four-lobed design such that each O-ring forms a four-pronged rectangular structure that forms a concave surface as it extends between each of the four prongs. Each of the O-rings are configured to make primary sealing contact at its lobes with an engagement surface, and compress, contour or expand as appropriate between the four lobes to form a full seal with a contact surface. FIG. 51B shows a cross-sectional view of an optional arrangement for the four-lobed O-rings have a more defined X-shape.

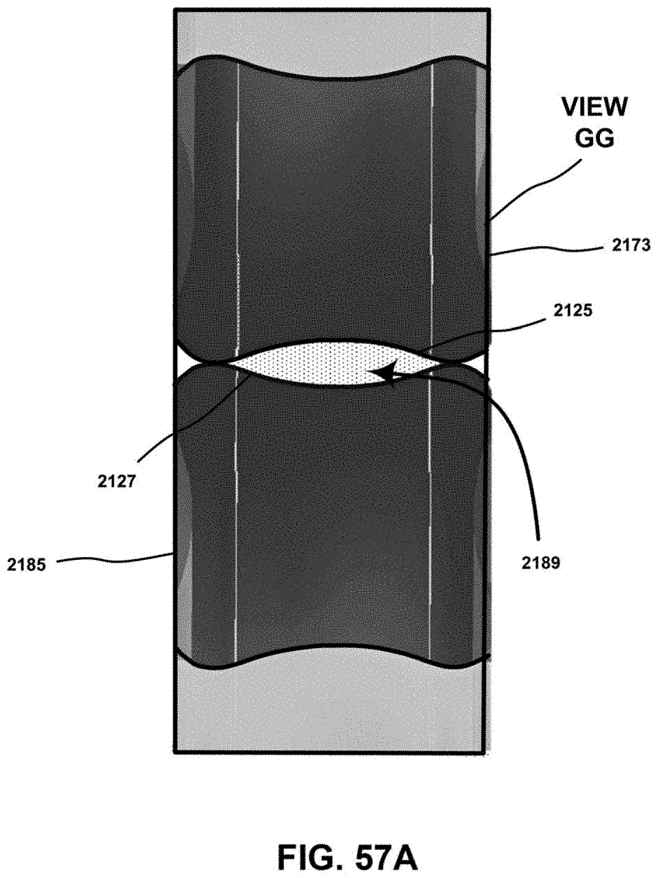

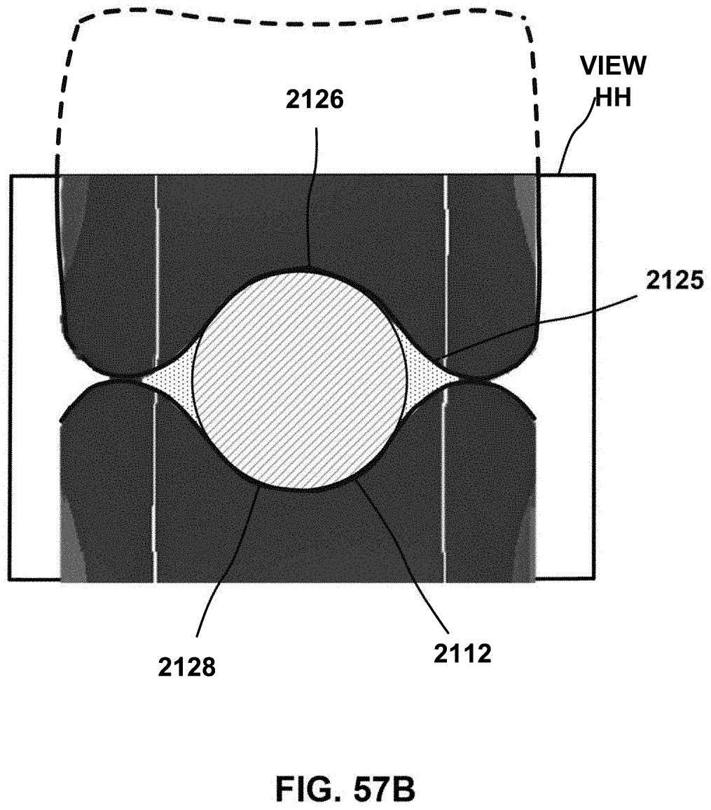

Referring now to FIGS. 57A and 57B, a cross-sectional view of the nip is show with and without an elongate device being shown. As can be seen in FIG. 57A, the first O-ring 2173 for the first roller drive forms a generally concave engagement surface 2125 for contacting the elongate device in its natural non-contact states, as does the second O-ring 2185 for the second roller drive. As can be seen best in FIG. 57B, when an elongate device is fed through the nip, the quad-type O-ring arrangements of engagement members readily form about most of the outer surface of the elongate device to form a high contact area grip about a greater area of the elongate device than most roller engagement members. Further, each quad-type O-ring envelops and surrounds a corresponding lateral region of the elongate device without imparting significant compressive forces against the elongate device. As shown in FIG. 57B, the quad-type O-ring expands laterally and inward toward the outer support surface of the roller rather than firmly maintaining its cross-sectional shape and imparting high compressive forces against the elongate device. The high contact area of engagement provided by the quad-type O-rings offsets the engagement benefits provided by high compressive force arrangements for engaging the elongate device. Further, the elastomeric material of the quad-type O-ring provides enhanced frictional contact engagement with the elongate device. As such, enhanced engaging contact can be formed with the elongate device through the nip without applying high compressive forces against the elongate device. Further, such an arrangement can significantly reduce rotational forces regarding for driving the drive rollers simply for engaging the elongate device within the nip.

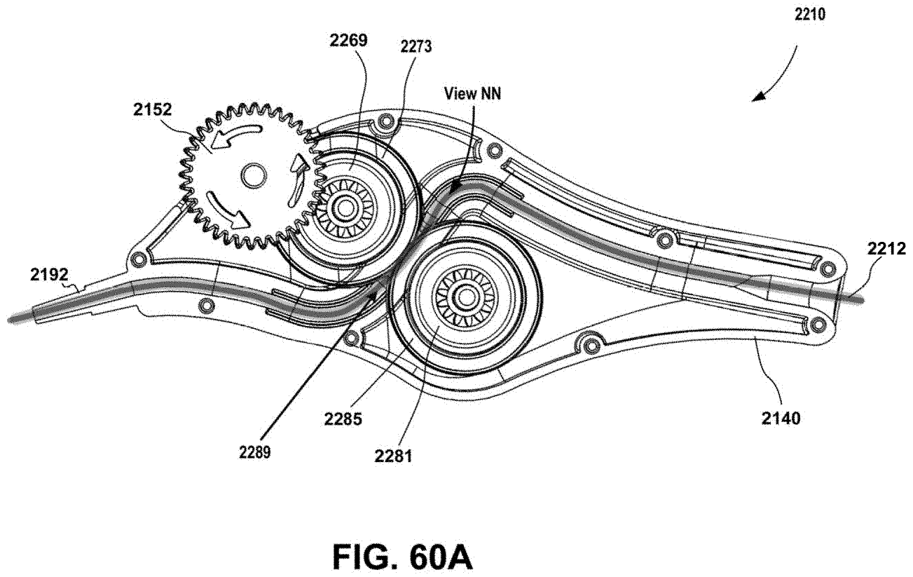

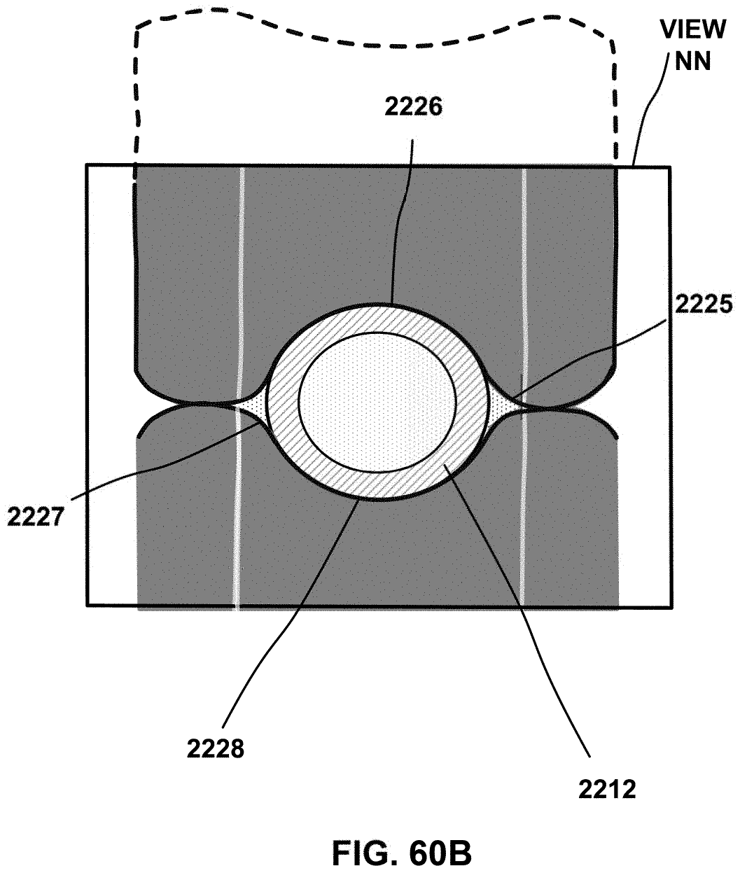

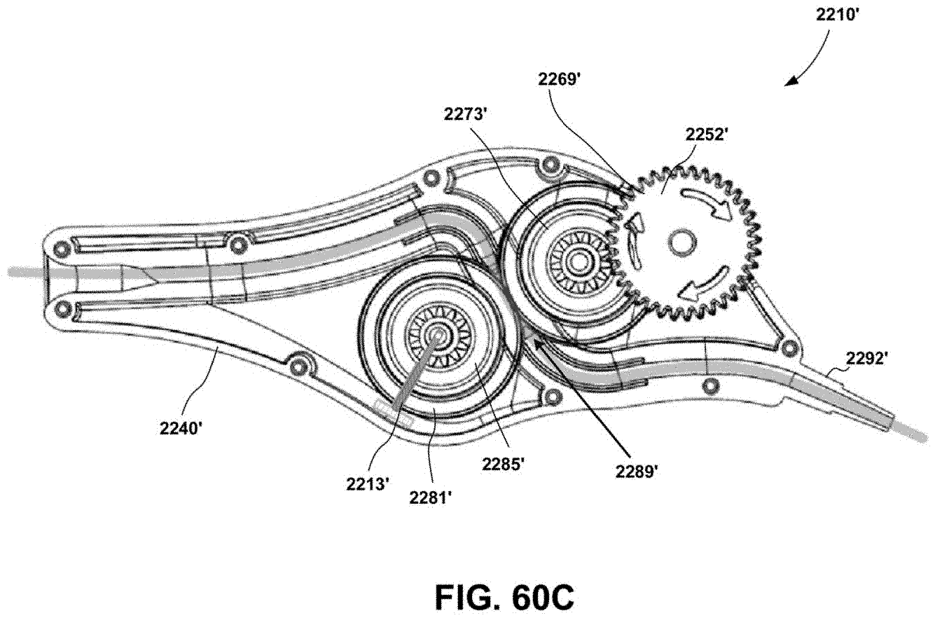

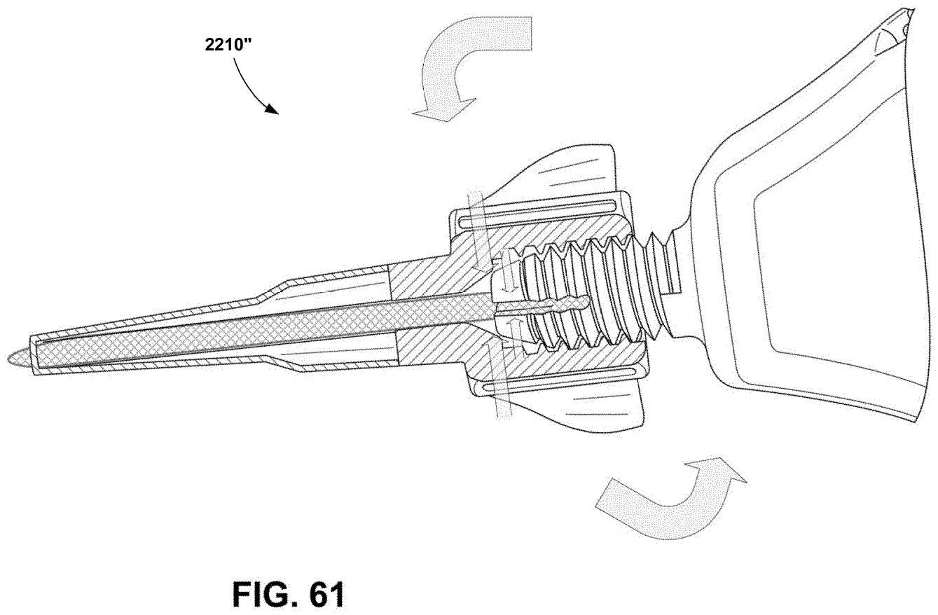

Referring now to FIGS. 60A and 60B, another arrangement of the advancer device is show along with an elongate device in the form of a catheter 2212 having a hollow center, which demonstrates the effectiveness for using a quad-type O-ring with the drive rollers and nip for engaging the elongate devices and translating the same through the advancer device. As shown in FIG. 61, an example advancer device according to aspects and features described herein can successfully translate and advance a tubular elongate device therethrough without collapsing the elongate device as would typically occur for conventional devices applying high compressive forces at nip or drive roller regions.

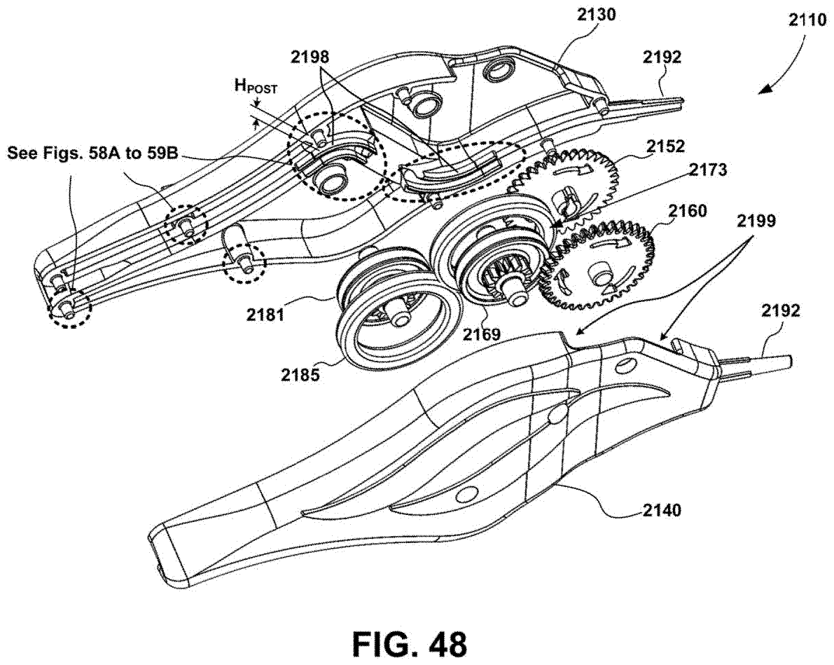

Referring now to FIGS. 60A to 60B, along with FIG. 48, cross-sectional views are shown through the enclosure along portions of the pathway 2120 for illustrating an offset male-female engagement arrangement of walls 2198 received in mating openings 2199 provided between the left and right housing along portions of the pathway at both sides of the pathway before and after the medial bend portion of the pathway. The male-female offset arrangement provides a barrier prior to the bend portion and the nip for blocking suture material or other fine materials that can be pulled by the elongate device from being caught within a seam or gap between left and right sides of the housing.

Referring now to FIG. 60C, an optional cross-sectional concept view is shown for an advancer device similar to other examples shown and described herein, which includes an adjustment rod 2313 configured to provide fine tune adjustments for the nip to allow different size and types of elongate devices to be used with the advancer device.

### Augmented Grip Connection & Reverse Clutch

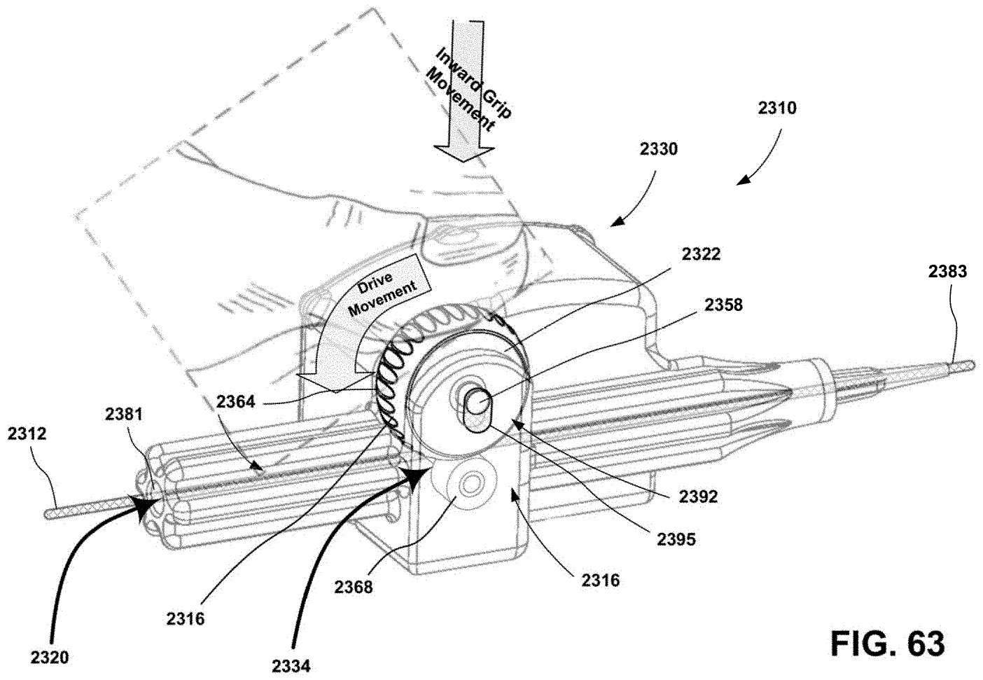

Referring now to FIGS. 63-65, an example drive-enhanceable, anti-slip handheld elongate medical device advancer 2310 is shown, which generally includes aspects and features similar to the example elongate device advancers discussed above along with operating in a similar manner, except as discussed hereafter. In particular, advancer 2310 includes aspects and features similar to advancer 1310 shown in FIG. 11 and discussed in greater detail above, but without including mechanical drive features. As such, like numbers generally refer to like features.

Advancer 2310 includes an advancer body 2330 that defines an elongate device pathway 2320 therethrough, such as for a guide wire or other elongate medical device 2312, which has an entry port 2381 or inlet at a proximal end portion and an exit port 2383 at an opposite distal end portion for advancing the elongate medical device 2312 therethrough and into an introducer pathway (not shown). The augmented advancer also includes a manual control thumbwheel 2322 partially embedded in the advancer body 2330, in which the thumbwheel is arranged to receive a user-exerted Drive Movement from a hand, and in particular from a thumb, for rotating the thumbwheel about a thumbwheel axis 2358. The drive augmentable advancer also includes a manual drive 2316 attached to the advancer body and operatively coupled with the thumbwheel 2322, in which the manual drive includes a nip 2334, a transmission 2364 in the form of a direct drive portion of the thumbwheel, and a reverse clutch 2392. The manual drive has a first roller (thumbwheel 2322) and an opposing second roller 2368 configured for jointly engaging opposite outer surface regions of the elongate medical device 2312 therebetween in an interference fit at a drive location along the pathway 2320.

The nip 2334 is configured to maintain a constant drive connection with the elongate medical device 2312 at the drive location when extending through the pathway 2320 and the drive location. The transmission connects the nip with the thumbwheel for transmitting a drive force to the elongate medical device at the drive location responsive to receiving the user-exerted rotational Drive Movement, and the reverse clutch 2392 that maintains and allows for the user to selectively augment a first interference grip connection between the nip and the elongate medical device when the elongate medical device extends through the drive location. The first interference grip connection transmits the drive force to the elongate medical device to advance the elongate device through the advancer and into the introducer pathway without slipping.

FIGS. 63 to 65 a depict a relatively simple, yet an incredibly effective augmentable-grip feature for use with elongate device advancer and especially for elongate medical device advancers, for which unexpected or unforeseen obstacles and challenges with advancing the elongate device during surgical support procedures can significantly impact surgical procedures. In many circumstances, challenges related to advancing a guide wire, catheter or other such elongate medical device can readily be overcome with the availability of enhanced or augmented grippability for a drive connection between an advancer and the elongate device being advanced. For the example depicted in FIGS. 63-65, an option for the user to press inward or downward on the manual thumbwheel currently being used for advancing the elongate device can enhance or augment the advancer grip connection sufficiently for overcoming a challenge encountered during advancement, and thereby readily mitigate the potential issue.

As shown in FIG. 63, the reverse clutch feature provide includes a slotted support 2395 for supporting rotation of the thumbwheel. The thumbwheel 2322 operates generally the same as it would if rotatably supported by a more typical pin or axle type round support, because an interference fit exists between the thumbwheel and second roller 2368, as well as with the elongate device 2312 being fed therethrough. As such, the thumbwheel 2322 is naturally biased to a first end (e.g., top portion for the example shown) for operating to advance the elongate device in a manner similar to conventional advancers that support a thumbwheel or similar drive wheel with a rounded pivot support. However, in the event of an obstruction, slippage or other significant need for an enhanced grip connection between the elongate device and the advancer, the reverse clutch, slotted support arrangement for the thumbwheel allows the user to apply inward force on the thumbwheel and thereby apply an inward grip movement to the thumbwheel concurrent with a rotation movement and thereby enhance grip force through the nip.

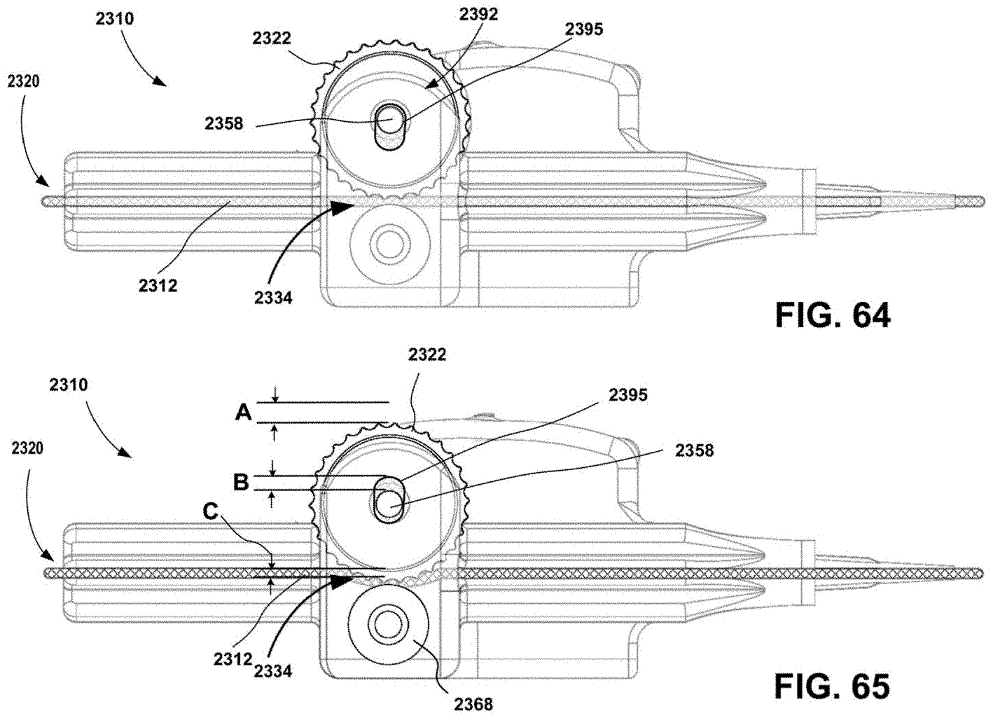

FIGS. 64 and 65 depict actions occurring when the user concurrently applies an inward grip movement to the thumbwheel 2322 along with applying rotational drive movements. Although an interference grip fit exists through the nip 2334 at the drive portion of the pathway, both the thumbwheel 2322 and the second roller 2368 have degrees of flexibility and compressibility for accommodating an augmented or enhanced application of grip connection force in a direction normal to nip rotations, which can allow for inward movement of the thumbwheel 2322 toward the nip in a nip tighten direction due to the slotted support arrangement of the reverse clutch 2392. Further, guide wires, catheters and most elongate medical devices typically being advanced generally include sufficient flexibility to accommodate increased flexion through the nip for at least a portion of their advancement. Thus, as depicted in FIG. 65, the user's inward grip force application significantly augments existing grip forces applied via the nip for providing enhanced grip to assist with advancing the elongate device on an as-needed basis.

The reverse clutch 2392 selectively increases the first interference grip connection to a greater second interference grip connection between the nip and the elongate medical device responsive to the thumbwheel receiving an inward user-exerted grip increase movement, which for the example of FIGS. 63-65 can be enabled via increased degrees of movement freedom on only one side of the nip (i.e., the thumbwheel). An overall combination of the same or different compressibility, durometer and related material and structural characteristics of the thumbwheel, second roller, elongate device, assembly, and the like can impact an amount of deflection actually applied to nip components and the elongate medical device within the nip. Generally, however, the grip connection through the nip is a function of the interference fit and friction through the nip such that an amount of normal force applied (or enhanced force applied) is the most significant factor rather than actual deflection at the nip. Nonetheless, as further shown in FIG. 65, differing deflections can apply for the inward grip movement applied (A) by the user in comparison with a deflection distance (B) for the thumbwheel axis within the slotted support, as well as for an actual deflection imparted to the elongate device within the nip (C).

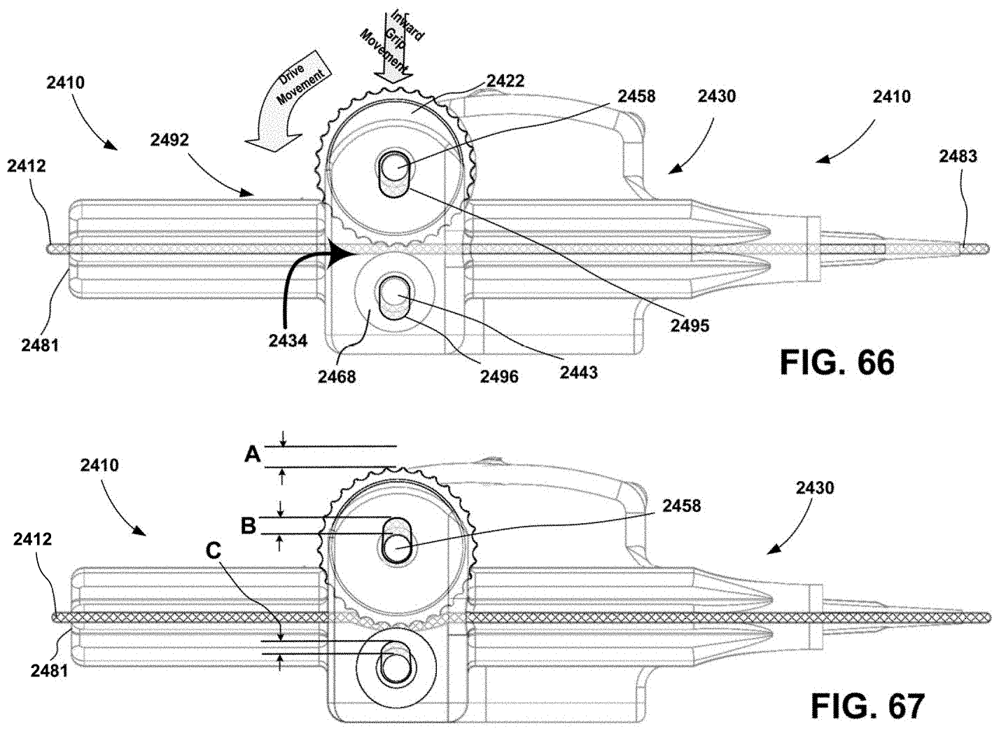

Referring now to FIGS. 66 and 67, a similar example drive force-augmentable, anti-slip handheld elongate medical device advancer 2410 is shown that is generally the same as advancer 2310 except as discussed hereafter regarding a slotted support and degrees of freedom for a second roller or idler roller of the nip. As such, like numbers generally refer to like features. Advancer 2410 differs from advancer 2310 discussed immediately above in that the second roller 2468 of the nip 2434 also includes a slotted support 2496 similar to the slotted support 2495 for the thumbwheel 2422, which is also aligned in a nip tighten direction extending normal to and across the nip 2434 and generally transverse to the pathway 2320 at the drive portion of the nip. As such, the reverse clutch 2492 of advancer 2410 further includes the slotted support 2496 for the second roller 2468 along with the slotted support 2495 of the combination thumbwheel/first roller 2422.

As generally discussed above in the previous example, even though degrees of movement freedom can be enabled as part of the overall functional arrangement and potential operations for the reverse clutch 2492 for various advancer arrangements, the related movement of components for an inward grip movement for augmented application of grip forces can impact other adjacent or affected components. As such, even though the second roller of the previous example lacked a slotted support, enhanced or augmented forces applied across the nip impacted the second roller and its resultant compression at the nip. Similarly for the example of FIGS. 66 and 67, in addition to the thumbwheel the second roller also has increased directional degrees of movement freedom across the nip related to reverse clutch operations, which can further enhance an amount of augmentation of the grip connection across the nip. In particular, both the first roller/thumbwheel 2422 and the second roller 2468 can be provided degrees of movement freedom in the same direction across the nip as applied forces and movement imparted across the nip from the user applying an inward grip movement. As such, both rollers across the nip can more freely compress about the elongate device within the nip and thereby potentially increase surface contact area along with applying a more balanced, bilateral augmented grip connection with the elongate device.

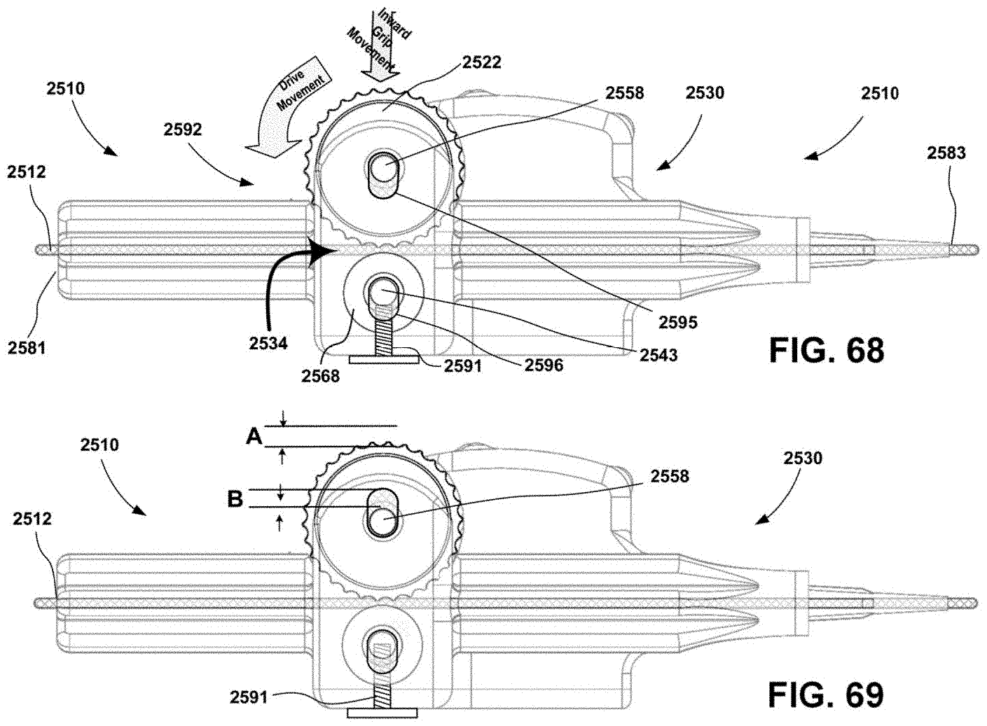

Thus, directional freedom of movement as can be applied via slotted supports when provided for both sides of a nip can enhance overall functionality and effectiveness of a reverse clutch feature. However, some advancer arrangements and implementations may not benefit from corresponding bilateral degrees of freedom across the nip, and/or can have other limitations and concerns for providing slotted supports for both sides of a nip or even in general. Further, it can be beneficial for various advancer arrangements to restrain use of a reverse clutch feature and only enable use of the same for certain conditions, procedures or amounts. Thus, in many circumstances, features of a reverse clutch and related actions can provide much greater benefits and overall effectiveness if some or all movement freedoms were limited or restrained for appropriate conditions or motions. FIGS. 68 and 69 depict an example for limiting degrees of freedom and/or adjusting movement freedom for a second roller, for instance.

Referring now to FIGS. 68 and 69, another related example drive-augmented, anti-slip handheld elongate medical device advancer 2510 that is similar to the examples of FIGS. 63-65 and 66-67 except as discussed hereafter regarding augmented grip advancers having locks, limiters and related mechanisms installed for restraining augmented grip and related functionality. As such, like numbers generally refer to like features. Advancer 2510 differs from advancers 2310 and 2410 discussed above in that the second roller 2568 of nip 2534 is locked or limited with respect to augmented grip movements or functionality based on a position lock/limiter 2591 being attached to the pin 2543 of the second roller 2568.

Lock/Limiter 2591 as shown can be attached to the second roller 2568 aligned with and proximate slotted support 2596 of the reverse clutch 2592 for controlling movement of the second roller with respect to augmented grip connection movements, for specific placement of the second roller with respect to these movements, and/or for limiting impacts of the movements with respect to the roller. Depending on the type, anticipated usage, and design of various advancers, one or more rollers and other drive related features can cooperate with or be included as part of nip or related advancement driver whose primary function and significance is for support, alignment and other drive related functions, which can be adversely impacted when moved even temporarily as pan of augmented grip connection movements.

For instance, idler rollers can often be integrated with nip components to perform significant non-drive functions, such as routing an elongate medical device through the pathway before or after the nip, for which significant displacement during augmented grip movements can be adversely impact. Further, various drive related rollers of an advancer can have different types of contact surfaces, durometer, compression and the like, for which an augmented grip movement could impart an excessive response with an unbalanced system or cause detrimental design changes. The use of lock or limiter supports can protect against such adverse impacts and can further provide supportive adjustments to better enable augmented grip movements. For example, advancer 2510 makes use of thumbwheel 2522 for receiving user-exerted movements including both rotation movements and inward augmented grip movements. As such, thumbwheel 2522 that acts as a drive roller has a much larger size than the second roller 2568, and most likely has a harder durometer with less compressibility than the second roller 2556 to provide desired thumb grip interactions and effective user controls.

Thus, thumbwheel 2522 directly receives user-exerted forces and movements, which could impart excessive force concentrations and overall impacts on the smaller, more compressible second roller along with causing unbalanced nip movements in the direction of the second roller —especially when the second roller includes a slotted support 2595 having degrees of freedom oriented transverse with the nip. Limiter 2591 appropriately limits degrees of freedom and moveability of the second roller 2568, such that advancer 2510 can effectively take advantage of benefits available to the user for making augmented grip movements as desired or appropriate while controlling or guiding such movements with respect to the advancer design, arrangement and other parameters including balancing compression about the elongate device through the nip for maintaining elongate device alignment through the nip.

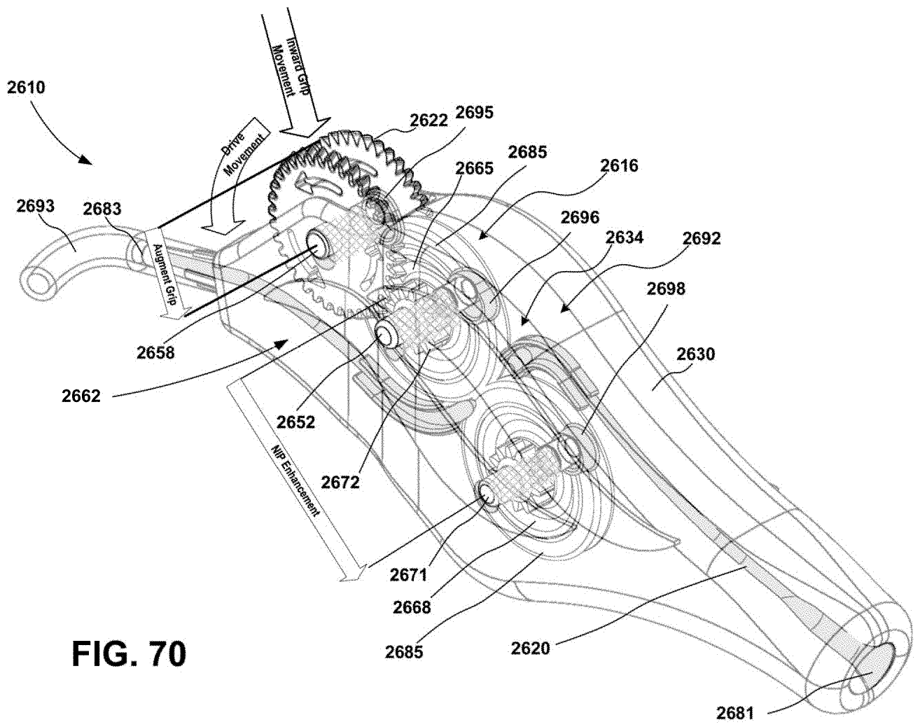

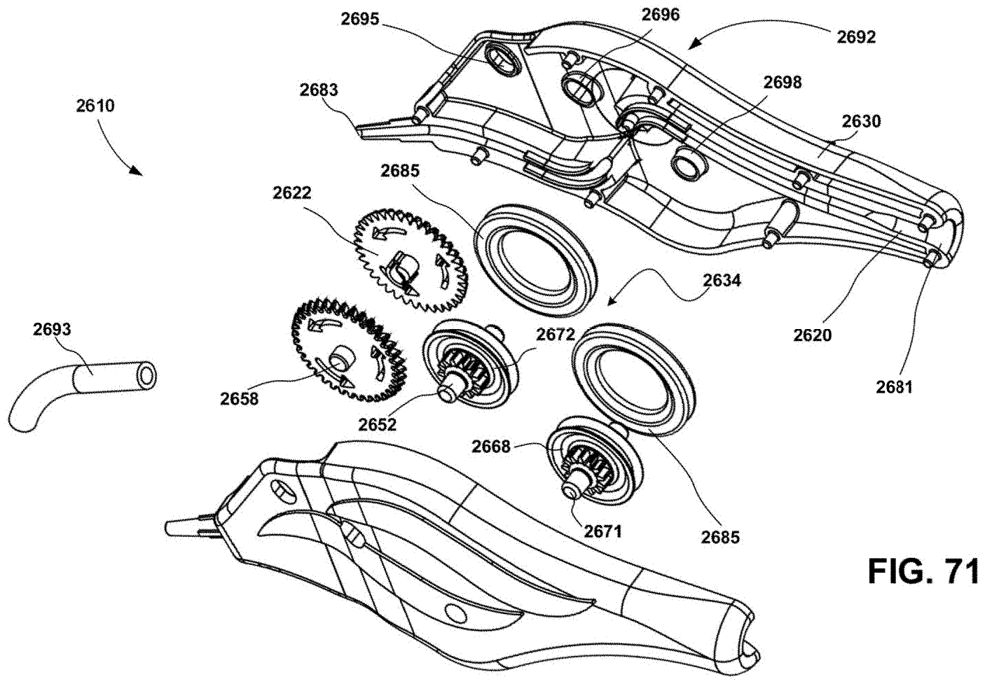

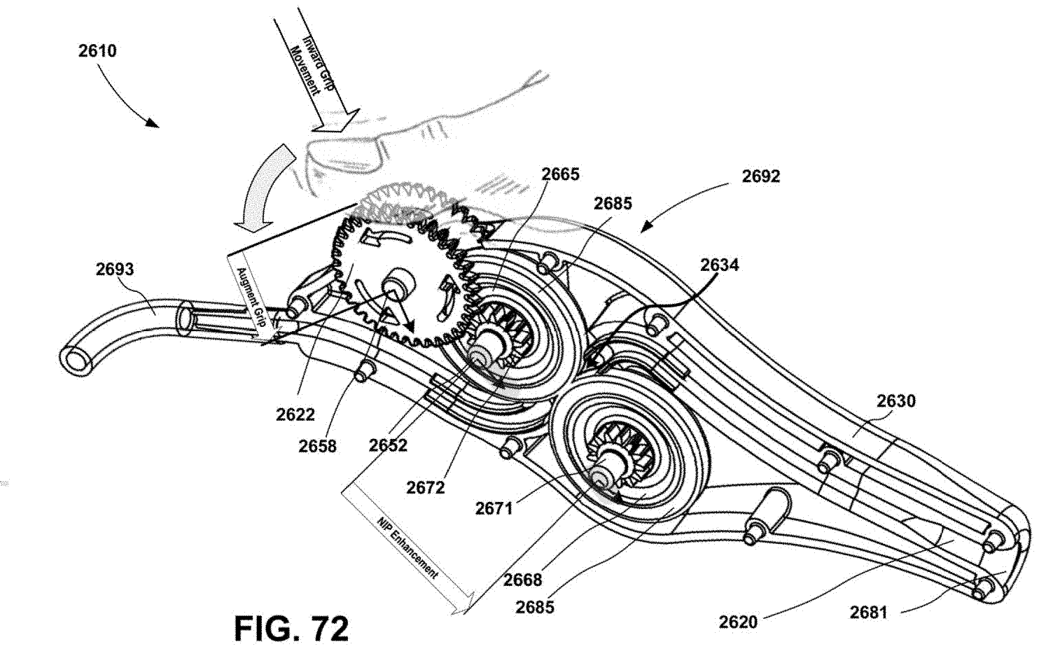

Referring now to FIGS. 70 to 76, an additional example drive-augmentable, anti-slip handheld elongate medical device advancer 2610 is shown that can generally be configured for being held in a single hand of a user along with being operated and controlled via the user's thumb. Advancer 2610 generally includes aspects and features discussed and described herein including along with previously described schematic depictions and examples, except as described hereafter. As such, like numbers generally refer to like features.

Advancer 2610 is configured and arranged for advancing a wide variety of elongate medical devices and for supporting many types of procedures in a wide range of environments including a multitude of surgical, inpatient, outpatient, curative and palliative procedures. Further, advancer 2610 can be used for advancing a wide range of elongate medical devices including catheters, guide wires, tubing, flexible needle members and the like, and for advancing into, within and through various connected introducer, surgical, diagnostic and related devices. As such, advancer 2610 is arranged as a manual advancer that can be controlled and operated within a wide range of functional uses for effectively advancing a corresponding elongate medical device (not shown) under most conditions without slipping or becoming jammed. In addition, advancer 2610 is arranged for readily accepting augmented control movements from the user along with performing advancement actions for permitting enhanced advancement functionality as desired.

As shown in FIG. 70, advancer 2610 generally includes an advancer body 2630 that defines an elongate medical device pathway 2620 extending therethrough from a proximal entry 2681 to a distal exit 2683, and a thumbwheel 2622 partially embedded in the advancer body proximate the distal end. The thumbwheel is arranged to receive a user-exerted Drive Movement from the user's hand, such as the thumb, for rotating the thumbwheel about it thumbwheel axis 2658. A manual drive 2616 is operatively coupled with the thumbwheel and includes a nip 2634, a transmission 2662, and a reverse clutch 2692. The nip includes a first roller 2665 and a second roller 2668 disposed at a drive location of the pathway 2620, which can jointly engage opposite outer surface regions of an elongate medical device 2612 (not shown) when it extends through the pathway and drive location in an interference fit between the rollers. As such, the nip can maintain a constant drive connection with the elongate medical device at the drive location. The transmission 2662 connects the nip with the thumbwheel for transmitting a user-exerted drive force to the elongate medical device at the drive location and includes a movement interface between the thumbwheel and the first roller 2665. The movement interface includes gear teeth formed along outer edge portions of the thumbwheel 2622, which drivingly engages a drive gear 2672 attached to and movable with the first roller 266. The movement interface is configured to move an axis 2652 of the first roller 2665 in the nip-tighten direction toward the nip when the thumbwheel moves in the grip direction inward away from the thumb.

The reverse clutch 2692 maintains the transmission drive connection between the thumbwheel and the nip including a first interference grip connection provided by the interference fit through the drive location, and is further configured for selectively augmenting the first interference grip connection between the nip and the elongate medical device. The first interference grip connection is configured to transmit the user-exerted drive force applied to the elongate medical device to advance the elongate medical device through the pathway and into the introducer pathway without slipping. The reverse clutch can selectively augment and increase the first interference grip connection to a higher second inherence grip connection between the nip and elongate medical device responsive to the thumbwheel receiving a user-exerted Inward Grip Movement. As depicted in FIG. 70, the user-exerted Grip Increase Movement can be applied to the thumbwheel along with the user applying a rotation Drive Movement. The Inward Grip Movement can be applied in an inward grip-increase direction or grip direction, such as generally perpendicular to a lateral support surface proximate the thumbwheel or generally perpendicular to the thumbwheel as indicated in FIG. 70.

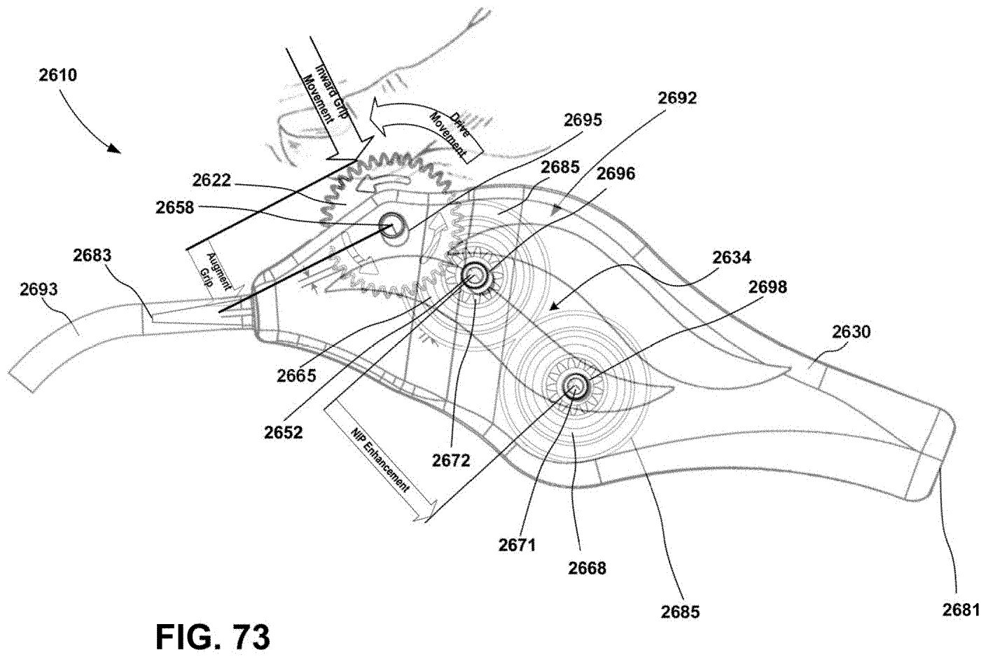

The reverse clutch 2692 can include a pair of thumbwheel rotation supports 2695 for rotatably supporting the thumbwheel 2622 on the advancer body, in which the thumbwheel slots are oriented in alignment with the grip direction. The reverse clutch can further include a pair of driver rotation slots 2696 oriented in a nip-tighten direction that can be oriented generally transverse to the nip at the drive location of the pathway. As best seen in FIG. 73, the pair of adjustable thumbwheel rotation supports 2695 can be configured for rotatably supporting the thumbwheel axis at an initial position on the advancer body corresponding with the first interference grip connection, for enabling inward movement of the thumbwheel to an augmented grip position responsive to a user-exerted grip increase movement, and for rotatably supporting the thumbwheel axis at the augmented grip position on the advancer body different from the initial position corresponding with the greater second interference grip connection. The thumbwheel 2622 is arranged to receive a user-exerted Inward Grip Movement from a thumb for increasing grip through the nip 2634 via movement from the thumb including a translation movement for moving the thumbwheel axis from the initial position to the augmented grip position. The Inward Grip Movement can include translation movement configured to increase compression of the compressible interface and thereby increase the interference grip connection of the nip from the first interference grip connection to the greater second interference grip connection for augmenting the drive force applied for advancing the elongate medical device.

The reverse clutch 2692 can further include a pair of adjustable driven rotation supports 2698 rotatably connecting an axis of the second roller 2668 with the advancer body. The pair of adjustable driven rotation supports 2698 can be configured for rotatably supporting the second roller axis 2671 at an initial position on the advancer body corresponding with the first interference grip, for enabling outward movement of the second roller away from the nip to an augmented grip position for bilaterally augmenting the interference grip, and for rotatably supporting the second roller axis at the augmented grip position on the advancer body different from the initial position corresponding with the greater second interference grip connection.

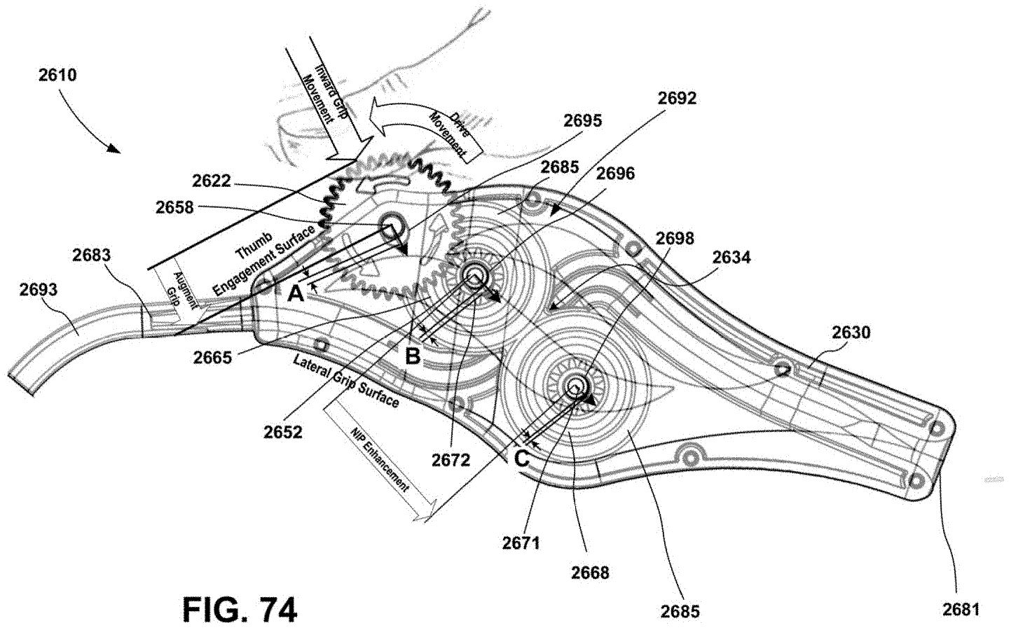

As best seen in FIGS. 74 to 76B, a compressible interface 2685 radially extends about both the first nip roller 2665 and the second roller 2668, which can bias the thumbwheel axis to the initial position and, when an Inward Grip Movement is exerted by the user, for being more tightly compressed about an elongate medical device (not shown) within the pathway for providing increased and bilateral compressive force that can augment grip with the elongate medical device. The pair of adjustable driver rotation supports 2696 can be oriented inward along the advancer body in a nip tighten direction, in which the driver slotted rotation supports rotatably support the drive roller 2665 at the initial position at a first end of the driver slotted rotation supports and at the augmented grip position disposed inward in the nip tighten direction along the driver slotted rotation supports. The pair of adjustable driven rotation supports 2698 can be oriented in a nip tighten direction, for which the driven slotted rotation supports rotatably support the second roller axis 2671 at the initial position at a first end of the driven slotted rotation supports and at the augmented grip position is disposed outward away from the nip along the nip tighten direction along the driven slotted pivot supports. The pair of adjustable thumbwheel rotation supports 2695 can include a pair of opposing, parallel slotted thumbwheel rotation supports oriented inward along the advancer body in an increase grip direction, in which the thumbwheel slotted rotation supports rotatably support the thumbwheel axis 2658 at the initial position at a first end of the pair of slotted thumbwheel rotation supports and at the augmented grip position disposed inward in the increase grip direction along the thumbwheel slotted rotation supports.

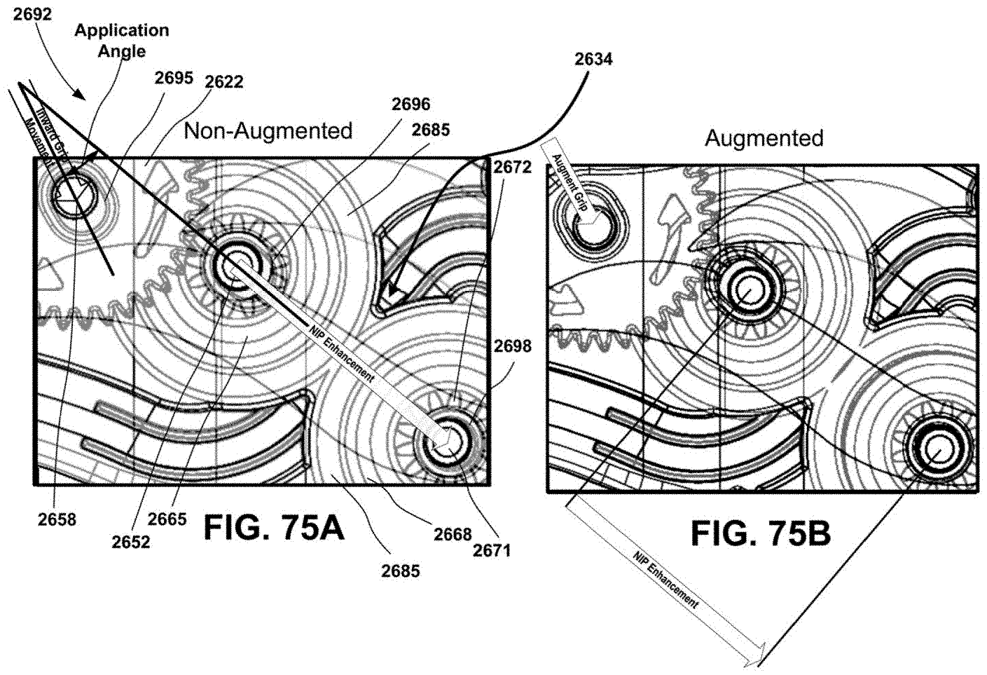

As shown in FIG. 75A, an Inward Grip Movement exerted by the user for moving the thumbwheel 2622 inward in a grip increase direction can form an Application Angle with a nip increase direction oriented normal to the nip 2634 in which grip increase movements and forces are applied. Further, as indicated in FIG. 74, the advancer body can define an upper lateral thumb engagement surface at the distal end portion for receiving the user's thumb and for user engagement with an exposed portion of the manual control during use of the advancer. The slotted thumbwheel rotation supports 2695 are disposed proximate the lateral thumb engagement surface and oriented inward and generally proximally along the advancer body away from the external thumb engagement surface. The increase-grip direction defines an acute angle with a proximal side of the external thumb engagement surface corresponding with flex movement of the user's thumb. The advancer body further can define a lower lateral grip surface at the distal end portion opposite from the upper lateral thumb engagement surface for receiving the user's fingers and gripping the advancer during use; and the nip-tighten direction can define an acute angle with the lower lateral grip surface corresponding with the user's grip. The increase-grip direction can define an acute angle with the nip-tighten direction, and the first roller can translate from the first position to the second position in the nip-tighten direction internally and distally along the advancer body from the first clutch direction.

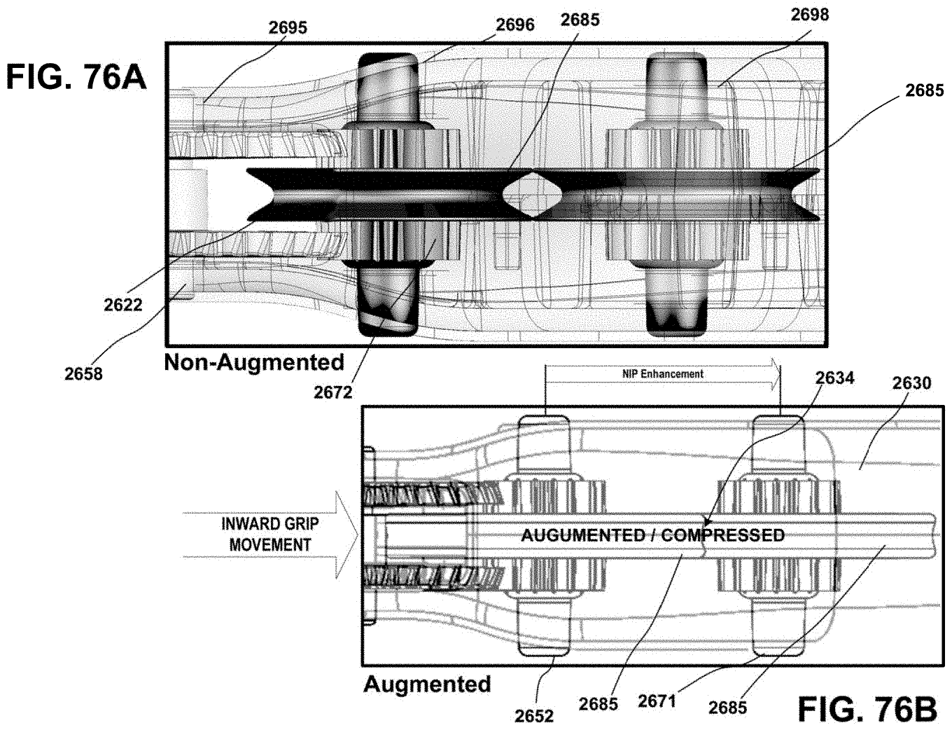

As best seen in FIGS. 75A to 76B, the first or driven drive roller 2665 can include a first outer region 2685 rotatable with the drive roller. A first outer engagement surface of the first outer region is configured to engage a first external radial portion of the elongate medical device, in which the first outer region 2685 can include a first compressible material configured to engage the first external radial portion in an interference relationship for the first interference grip, where the first compressible material is compressed during engagement of the first drive roller with the elongate device. The reverse clutch 2692 can include a first compressed portion of the first compressible material disposed in the interference relationship for the first interference grip, and the first compressed portion can have a first compressed state for the first interference grip connection and a second compressed state greater than the first compressed state for the second interference grip connection.

The second roller or driven roller 2668 can likewise include a second outer region 2685 rotatable with the second drive roller, and a second outer engagement surface of the second outer region can be configured to engage a second external radial portion of the elongate medical device. The second outer region can include a second compressible material configured to engage the second external radial portion in an interference relationship, where the second compressible material is compressed during engagement of the second drive roller with the elongate device. The reverse clutch can include a third compressed portion of the second compressible material disposed in the interference relationship for the first interference grip connection and a fourth compressed state greater than the third compressed state for the second interference grip connection. The first compressible material can be compressed a greater amount than the second compressible material for engagement of the reverse clutch for the increased second interference grip connection versus the first interference grip connection. The first and the second compressible materials can have substantially the same compressibility, and the second compressible materials have substantially different compressibility properties.

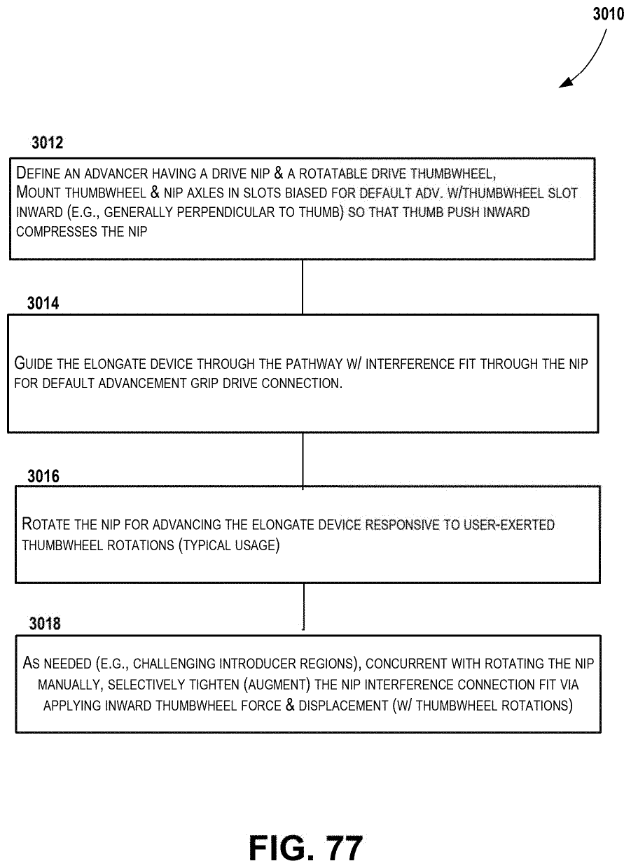

Referring now to FIG. 77 along with FIGS. 70-76B, a method 3010 is schematically represented for selectively increasing grip between an elongate medical device advancer and an elongate medical device extending through the advancer. The method can include defining 3012 an elongate device guided pathway through an advancer enclosure having a nip for advancing the elongate device operatively coupled with a manually rotatable thumbwheel for driving nip rotations, and the thumbwheel can be arranged to apply inward grip movements to the nip for enhancing interference grip with the elongate device through the nip. The method can also include guiding 3014 the elongate device through the pathway including establishing an interference fit between the elongate device and the nip for providing an advancement grip connection with the elongate device. The method can further include rotating 3016 the nip for advancing the elongate device responsive to user-exerted thumbwheel rotations for advancing the elongate device. In addition, the method can include, concurrent with rotating the nip, selectively tightening 3018 the nip interference advancement connection with the elongate device responsive to receiving user-exerted grip movement and forces applied to the thumbwheel along with thumbwheel rotations.

Defining 3012 the elongate device guided pathway can include arranging the nip and a drive portion of the pathway at a nip-tighten angle substantially perpendicular to the nip at an acute angle from a direction of inward grip movements for the thumbwheel for enabling effective advancement of the elongate device along the pathway along with providing an ergonomic advancer arrangement that can be gripped and controlled by a single hand of the user. Guiding 3014 and, in particular, establishing the interference fit can include establishing an interference fit that can advance the elongate device through the pathway and introduce the elongate device into a target pathway. Selectively tightening 3018 the nip interference advancement connection can include increasing the nip interference connection as needed for traversing the target pathway.

Although various embodiments have been described as having particular features and/or combinations of components, other embodiments are possible having a combination of any features and/or components from any of embodiments as discussed above. Aspects have been described in the general context of medical devices, and more specifically surgical instruments, but inventive aspects are not necessarily limited to use in medical devices.

1. An augmented, anti-slip handheld advancer for an elongate medical device, the advancer comprising:
an advancer body defining a pathway for the elongate medical device extending between an inlet at a proximal end portion and an exit at an opposite distal end portion for advancement into an introducer pathway;
a manual control thumbwheel partially embedded in the advancer body, the manual control thumbwheel arranged to receive a user-exerted drive movement from a hand for rotating the thumbwheel about a thumbwheel axis;
a manual drive attached to the advancer body and operatively coupled with the thumbwheel, the manual drive comprising:
a nip having a first roller and an opposing second roller configured for jointly engaging opposite outer surface regions of the elongate medical device therebetween in an interference fit at a drive location along the pathway, the nip configured to maintain a constant drive connection with the elongate medical device at the drive location when extending through the pathway and the drive location;
a transmission connecting the nip with the thumbwheel for transmitting a drive force to the elongate medical device at the drive location responsive to receiving the user-exerted rotational drive movement; and
a reverse clutch maintaining and selectively augmenting a first interference grip connection between the nip and the elongate medical device when the elongate medical device extends through the drive location, the first interference grip connection transmitting the drive force to the elongate medical device to advance the elongate device through the advancer and into the introducer pathway without slipping, the reverse clutch selectively increasing the first interference grip connection to a greater second interference grip connection between the nip and the elongate medical device responsive to the thumbwheel receiving an inward user-exerted grip increase movement.

2. The augmented, anti-slip handheld advancer of claim 1, the reverse clutch comprising:
a pair of adjustable thumbwheel rotation supports rotatably connecting the thumbwheel axis with the advancer body, the pair of adjustable thumbwheel supports configured for rotatably supporting the thumbwheel axis at an initial position on the advancer body corresponding with the first interference grip connection, enabling inward movement of the thumbwheel to an augmented grip position responsive to a user-exerted grip increase movement, and for rotatably supporting the thumbwheel axis at the augmented grip position on the advancer body different from the initial position corresponding with the greater second interference grip connection.

3. The augmented, anti-slip handheld advancer of claim 2, the reverse clutch further comprising:
a pair of adjustable driver rotation supports rotatably connecting an axis of the first roller with the advancer body, the pair of adjustable driver rotation supports configured for rotatably supporting the first roller axis at an initial position on the advancer body corresponding with the first interference grip, enabling movement of the first roller to an augmented grip position responsive to movement of the thumbwheel, and for rotatably supporting the first roller axis at the augmented grip position on the advancer body different from the initial position corresponding with the greater second interference grip connection;
an interface maintaining a drive gap between the thumbwheel axis and the first roller axis and configured to move the first roller axis from the initial position to the augmented grip position when the thumbwheel axis is moved from the initial position to the augmented grip position by the user; and
a compressible interface radially extending about the first roller, the compressible interface biasing the first roller axis and the thumbwheel axis to the initial positions based on the interface and providing compressive force for augmenting grip with the elongate medical device;
wherein the thumbwheel is arranged to receive a user-exerted grip increase movement from the thumb including a translation movement for moving the thumbwheel axis from the initial position to the augmented grip position, the translation movement configured to increase compression of the compressible interface and thereby increase the interference grip connection of the nip from the first interference grip connection to the greater second interference grip connection for augmenting the drive force applied to advance the elongate medical device.

4. The augmented, anti-slip handheld advancer of claim 2, the reverse clutch further comprising:
a pair of adjustable driven rotation supports rotatably connecting an axis of the second roller with the advancer body, the pair of adjustable driven rotation supports configured for rotatably supporting the second roller axis at an initial position on the advancer body corresponding with the first interference grip, enabling outward movement of the second roller away from the nip to an augmented grip position for bilaterally augmenting the interference grip, and for rotatably supporting the second roller axis at the augmented grip position on the advancer body different from the initial position corresponding with the greater second interference grip connection; and
a compressible interface radially extending about the second roller, the compressible interface biasing the thumbwheel axis to the initial position for providing increased and bilateral compressive force for augmenting grip with the elongate medical device.

5. The augmented, anti-slip handheld advancer of claim 2, the reverse clutch further comprising:
a pair of adjustable driver rotation supports rotatably connecting an axis of the first roller with the advancer body, the pair of adjustable driver rotation supports configured for rotatably supporting the first roller axis at an initial position on the advancer body corresponding with the first interference grip, enabling inward movement of the first roller to an augmented grip position responsive to a user-exerted grip increase movement, and for rotatably supporting the first roller axis at the augmented grip position on the advancer body different from the initial position, the augmented grip position corresponding with the greater second interference grip connection; and
an interface maintaining a drive gap between the thumbwheel axis and the first roller axis and configured to move the first roller axis from the initial position to the augmented grip position when the thumbwheel axis moves from the initial position to the augmented grip position;
wherein:
the pair of adjustable thumbwheel rotation supports comprises a pair of opposing, parallel slotted thumbwheel rotation supports oriented inward along the advancer body in an increase grip direction, the thumbwheel slotted rotation supports rotatably supporting the thumbwheel axis at the initial position at a first end of the pair of slotted thumbwheel rotation supports and at the augmented grip position disposed inward in the increase grip direction along the thumbwheel slotted rotation supports; and
the pair of adjustable driver rotation supports comprises a pair of opposing, parallel slotted driver rotation supports oriented inward along the advancer body in a nip tighten direction, the driver slotted rotation supports rotatably supporting the first roller axis at the initial position at a first end of the driver slotted rotation supports and at the augmented grip position disposed inward in the nip tighten direction along the driver slotted rotation supports.

6. The advancer of claim 5, wherein:
the advancer body defines an upper lateral thumb engagement surface at the distal end portion for receiving the user's thumb and for user engagement with an exposed portion of the manual control during use of the advancer;
the slotted thumbwheel rotation supports are disposed proximate the lateral thumb engagement surface oriented inward and generally proximally along the advancer body away from the external thumb engagement surface; and
the increase-grip direction defines an acute angle with a proximal side of the external thumb engagement surface corresponding with flex movement of the user's thumb.

7. The advancer of claim 6, wherein:
the advancer body further defines a lower lateral grip surface at the distal end portion opposite from the upper lateral thumb engagement surface for receiving the user's fingers and gripping the advancer during use; and
the nip-tighten direction defines an acute angle with the lower lateral grip surface corresponding with the user's grip.

8. The advancer of claim 7, wherein:
the increase-grip direction defines an acute angle with the nip-tighten direction; and
the first roller translates from the first position to the second position in the nip-tighten direction internally and distally along the advancer body from the first clutch direction.

9. The augmented, anti-slip handheld advancer of claim 5, further comprising:
a slot limiter attached to the advancer body for at least one of the thumbwheel and driver slotted rotation supports, the slot limiter permitting the user to at least one of limit a length of the corresponding thumbwheel or driver slotted rotation supports or to set a position of the corresponding thumbwheel or driver slotted rotation support.

10. The augmented, anti-slip handheld advancer of claim 4, wherein:
the pair of adjustable driver rotation supports comprises a first pair of opposing, parallel slotted driver rotation supports oriented inward along the advancer body in a nip tighten direction, the driver slotted rotation supports rotatably supporting the drive roller at the initial position at a first end of the driver slotted rotation supports and at the augmented grip position disposed inward in the nip tighten direction along the driver slotted rotation supports; and
the pair of adjustable driven rotation supports comprises a pair of opposing, parallel slotted driven rotation supports oriented in a nip tighten direction, the driven slotted rotation supports rotatably supporting the second roller axis at the initial position at a first end of the driven slotted rotation supports and at the augmented grip position disposed outward away from the nip along the nip tighten direction along the driven slotted pivot supports.

11. The augmented, anti-slip handheld advancer of claim 10, further comprising:
a slot limiter attached to the advancer body for at least one of the driver slotted rotation supports and the driven slotted rotation supports, the slot limiter permitting the user to at least one of limit a length of the corresponding driver slotted rotation supports or driven slotted rotation supports, or to set a position of the corresponding driver slotted rotation supports or the driven slotted rotation supports.

12. The augmented, anti-slip handheld advancer of claim 2, wherein:
the drive roller further comprises a first outer region rotatable with the drive roller;
a first outer engagement surface of the first outer region is configured to engage a first external radial portion of the elongate medical device;
the first outer region includes a first compressible material configured to engage the first external radial portion in an interference relationship for the first interference grip, wherein the first compressible material is compressed during engagement of the first drive roller with the elongate device; and
the reverse clutch includes a first compressed portion of the first compressible material disposed in the interference relationship for the first interference grip, the first compressed portion having a first compressed state for the first interference grip connection and a second compressed state greater than the first compressed state for the second interference grip connection.

13. The augmented, anti-slip handheld advancer of claim 12, wherein:
the second roller further comprises a second outer region rotatable with the second drive roller;
a second outer engagement surface of the second outer region is configured to engage a second external radial portion of the elongate medical device;
the second outer region includes a second compressible material configured to engage the second external radial portion in an interference relationship, wherein the second compressible material is compressed during engagement of the second drive roller with the elongate device; and
the reverse clutch includes a third compressed portion of the second compressible material disposed in the interference relationship for the first interference grip connection and a fourth compressed state greater than the third compressed state for the second interference grip connection.

14. The augmented, anti-slip handheld advancer of claim 13, wherein:
the first compressible material is compressed a greater amount than the second compressible material for engagement of the reverse clutch for the increased second interference grip connection versus the first interference grip connection.

15. The augmented, anti-slip handheld advancer of claim 14, wherein the first and the second compressible materials have substantially the same compressibility.

16. The augmented, anti-slip handheld advancer of claim 14, wherein the first and the second compressible materials have substantially different compressibility properties.

17. A handheld advancer for advancing an elongate medical device using a single hand, the advancer comprising:
an advancer body defining:
a pathway for the elongate medical device extending between an inlet at a proximal end portion and an exit at an opposite distal end portion;
an upper palm engagement surface having a proximal palm rest and a distal thumb engagement region at an obtuse angle from the palm rest; and
a lower finger grip region opposite the upper palm engagement surface and distal thumb engagement region, the upper palm engagement surface and the lower finger grip region configured for ergonomic single hand grip of the advancer and user control of the advancer through intuitive distal thumb roll movements for distal advancement of the elongate medical device and through inward application of augmented grip forces for increasing advancer grip with the elongate medical device;
a manual control thumbwheel partially embedded in the advancer body distal end within the thumb engagement region, the thumbwheel arranged to receive the distal thumb roll movements to advance the elongate medical device distally and receive the inward application of thumb grip forces for enhancing advancer grip with the elongate medical device; and
a manual drive attached to the advancer body and operatively coupled with the thumbwheel, the manual drive comprising:
a nip having a first roller and an opposing second roller configured for jointly engaging opposite outer surface regions of the elongate medical device therebetween at a drive location along the pathway, the nip configured to maintain a constant translation drive connection with the elongate medical device at the drive location when the elongate medical device extends through the drive location;
a transmission connecting the nip with the manual control for transmitting a distal advancement translation force to the elongate medical device at the drive location responsive to user-exerted distal thumb roll movement of the thumbwheel; and
a reverse clutch maintaining and selectively augmenting an interference grip connection with the elongate medical device when extending through the nip at the drive location, the first interference grip connection transmitting the drive force to the elongate medical device to advance the elongate device through the advancer without slipping and into the introducer, the reverse clutch selectively increasing the first interference grip connection to a greater second interference grip connection between the nip and the elongate medical device responsive to the thumbwheel receiving an inward user-exerted grip increase movement for avoiding slip conditions and configured to apply the application of grip forces to the nip for increasing the interference grip connection responsive to the user-exerted inward application of grip forces to the thumbwheel;
wherein the reverse clutch is configured to apply the inward application of grip forces received in a grip direction from the thumbwheel to the nip at a nip-tighten direction angled away from the grip direction.

18. The advancer of claim 17, further comprising:
a pair of thumbwheel rotation slots rotatably supporting the thumbwheel on the advancer body, the first pair of pivot slots oriented in the grip direction substantially perpendicular with the thumb;
a pair of driver rotation slots rotatably supporting a first roller of the nip on the advancer body, the pair of driver rotation slots oriented in the nip-tighten direction substantially perpendicular to the nip; and
a movement interface between the thumbwheel and the first roller, the movement interface configured to move an axis of the first roller in the nip-tighten direction toward the nip when the thumbwheel moves in the grip direction inward away from the thumb;
wherein the grip direction and the nip-tighten direction form an acute angle therebetween.

19. A method for selectively increasing grip between an elongate medical device advancer and an elongate medical device extending through the advancer, the method comprising:
defining an elongate device guided pathway through an advancer enclosure having a nip for advancing the elongate device operatively coupled with a manually rotatable thumbwheel for driving nip rotations, the thumbwheel arranged to apply inward grip movements to the nip for enhancing interference grip with the elongate device through the nip;
guiding the elongate device through the pathway including establishing an interference fit between the elongate device and the nip for providing an advancement grip connection with the elongate device;
rotating the nip for advancing the elongate device responsive to user-exerted thumbwheel rotations for advancing the elongate device; and
concurrent with rotating the nip, selectively tightening the nip interference advancement connection with the elongate device responsive to receiving user-exerted grip movement and forces applied to the thumbwheel along with thumbwheel rotations.

20. The method according to claim 19, wherein:
defining the elongate device guided pathway includes arranging the nip and a drive portion of the pathway at a nip-tighten angle substantially perpendicular to the nip at an acute angle from a direction of inward grip movements for the thumbwheel for enabling effective advancement of the elongate device along the pathway along with providing an ergonomic advancer arrangement that can be gripped and controlled by a single hand of the user;
establishing the interference fit includes establishing an interference fit that can advance the elongate device through the pathway and introduce the elongate device into a target pathway; and
selectively tightening the nip interference advancement connection includes increasing the nip interference connection as needed for traversing the target pathway.

21-58. (canceled)
